(19) United States
(12) Patent Application Publication
(10) Pub. No.: US 2022/0041630 A1
(43) Pub. Date: Feb. 10, 2022

(54) PROCESSES FOR THE PREPARATION OF ARGINASE INHIBITORS AND THEIR SYNTHETIC INTERMEDIATES

(71) Applicant: Incyte Corporation, Wilmington, DE (US)

(72) Inventors: Joseph SCLAFANI, Wilmington, DE (US); Anthony J. Tomaine, Wilmington, DE (US); Robert J. Wilson, Wilmington, DE (US); Daniel Carper, Wilmington, DE (US); Wayne Han, Wilmington, DE (US); George Shi, Hangzhou Sandun (CN); Feiquiang Sun, Hangzhou Sandun (CN); Guobiao Lu, Hangzhou Sandun (CN); Jack Liang, San Francisco, CA (US)

(73) Assignee: Incyte Corporation, Wilmington, DE (US)

(21) Appl. No.: 17/305,932

(22) Filed: Jul. 16, 2021

Related U.S. Application Data
(60) Provisional application No. 63/089,293, filed on Oct. 8, 2020.
(60) Provisional application No. 63/053,482, filed on Jul. 17, 2020.

(51) Int. Cl. C07F 5/05; C07C 67/08; C07C 67/343; C07D 309/12; C07D 405/12; C07D 207/12; C07F 7/18; C07D 207/16; C07D 403/12; C07D 403/14; C07F 5/04; C07F 5/02

(52) U.S. Cl. CPC C07F 5/05; C07C 67/08; C07C 67/343; C07D 309/12; C07D 405/12; C07F 5/025; C07F 7/188; C07D 207/16; C07D 403/12; C07D 403/14; C07F 5/04; C07D 207/12

(57) ABSTRACT

Provided herein are processes and intermediates useful for the preparation of certain compounds, including a compound of formula 21 or formula 22

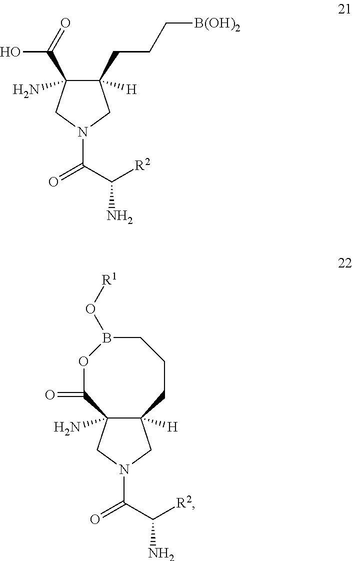

or a pharmaceutically acceptable salt of either.

### 1. BACKGROUND

Compounds of general formula 21 and formula 22

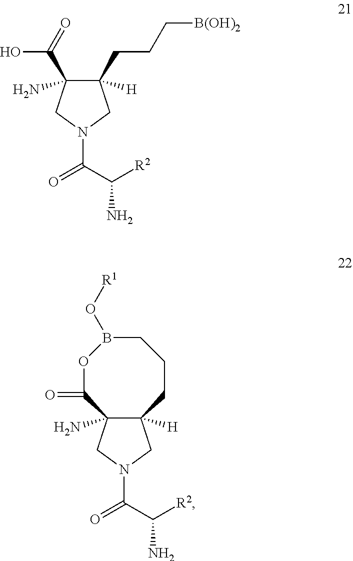

including pharmaceutically acceptable salts thereof, exhibit potent arginase inhibition.

U.S. Pat. Nos. 10,065,974 and 10,494,339 describe benchtop preparation of compounds of the general formula 21. U.S. Pat. No. 10,287,303 describes preparation of compounds of the general formula 22. A need exists for economical synthetic procedures and intermediate compounds for the preparation of the compounds of formula 21 and formula 22 including pharmaceutically acceptable salts thereof for batch process scale-up. Such alternative synthetic procedures and intermediate compounds are provided herein.

### 2. SUMMARY

Provided herein is a process for preparing a compound of formula 21:

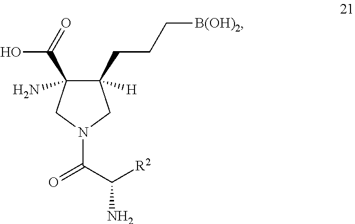

or a pharmaceutically acceptable salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

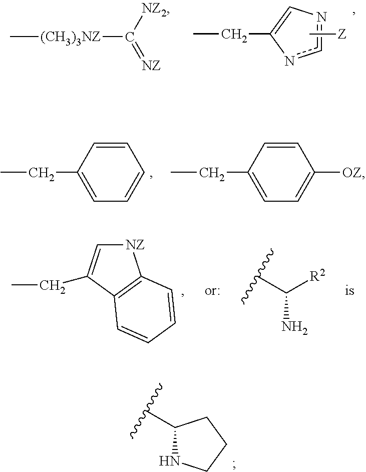

and
each Z is independently H, (C₁-C₆)alkyl, or a suitable protecting group.

Also provided herein is a process for preparing a compound of formula 22:

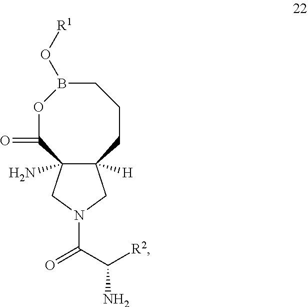

or a pharmaceutically acceptable salt thereof, wherein:
R¹ is H or (C₁-C₆)alkyl;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

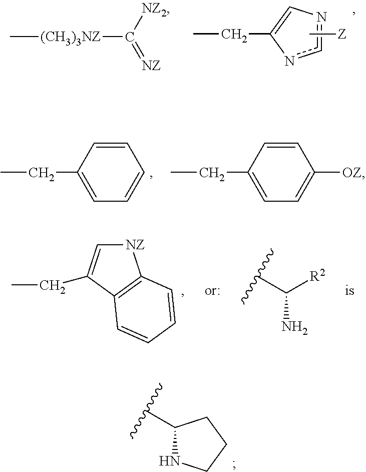

and
each Z is independently H, (C₁-C₃)alkyl, or a suitable protecting group.

In some embodiments, the process for preparing a compound of formula 21 or formula 22 comprises treating a compound of formula 20

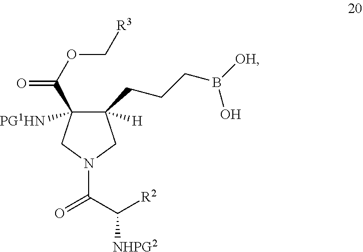

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

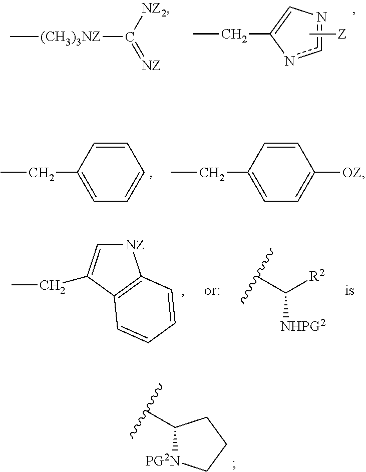

each Z is independently H, (C₁-C₃)alkyl, or a suitable protecting group;
R³ is a substituted or unsubstituted ring selected from aryl and heteroaryl; and
PG¹ and PG² are independently suitable amine protecting groups,
   under suitable conditions to deprotect and optionally condense the compound of formula 20 to form the compound of formula 21 or formula 22 or a pharmaceutically acceptable salt thereof.

In some embodiments, the suitable conditions to deprotect the compound of formula 20 comprise treating with hydrogen and a palladium catalyst.

In some embodiments, the process for preparing a compound of formula 21 or formula 22 further comprises preparing the compound of formula 20, the process comprising hydrolyzing the boronate of a compound of formula 19

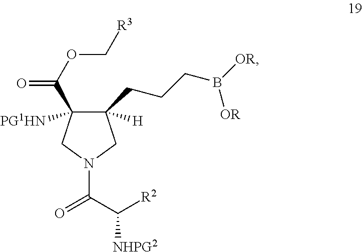

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

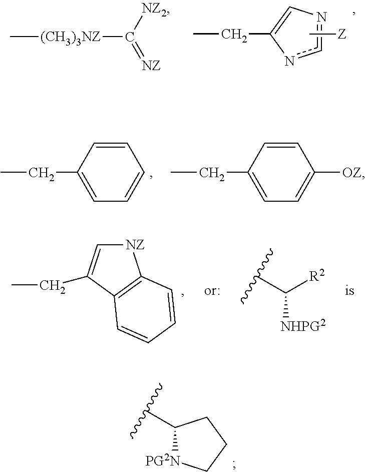

each Z is independently H, (C₁-C₃)alkyl, or a suitable protecting group;
R³ is a substituted or unsubstituted ring selected from aryl and heteroaryl; and
PG¹ and PG² are independently suitable amine protecting groups,
   under suitable conditions to hydrolyze the boronate of the compound of formula 19 to form the compound of formula 20 or a salt thereof.

In some embodiments, the suitable conditions to deprotect the compound of formula 19 comprise treating with sodium periodate. Without limitation, also provided herein is a process to prepare a compound of formula 20 from a compound of formula 19 as described herein.

In some embodiments, the process for preparing a compound of formula 21 or formula 22 further comprises preparing the compound of formula 19, the process comprising hydroborating a compound of formula 18

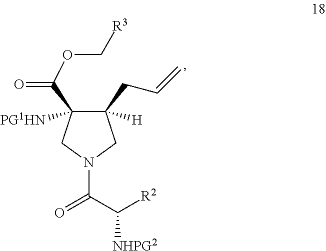

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

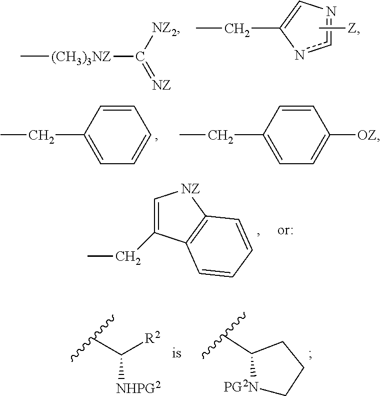

each Z is independently H, (C₁-C₃)alkyl, or a suitable protecting group;
R³ is a substituted or unsubstituted ring selected from aryl and heteroaryl; and
PG¹ and PG² are independently suitable amine protecting groups,
   under suitable conditions to hydroborate the compound of formula 18 to form the compound of formula 19 or a salt thereof.

In some embodiments, the suitable conditions to hydroborate the compound of formula 18 comprise treating with pinacol borane and an iridium catalyst. Without limitation, also provided herein is a process to prepare a compound of formula 19 from a compound of formula 18 as described herein.

In some embodiments, the process for preparing a compound of formula 21 or formula 22 further comprises preparing the compound of formula 18, the process comprising coupling a compound of formula 17

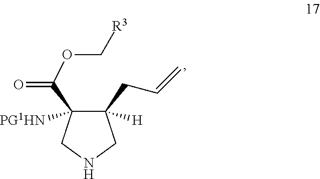

or a salt thereof, wherein:
R³ is a substituted or unsubstituted ring selected from aryl and heteroaryl; and
PG¹ is a suitable amine protecting group,
   with a compound of formula 16

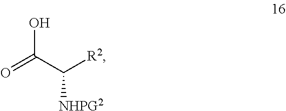

or salt thereof, wherein:
R² is H, (C₁-C₆)alkyl, —CH₂OZ, —CH(CH₃)OZ, —CH₂SZ, ˜(CH₂)₂SCH₃, —CH₂CONZ₂, —(CH₂)₂CONZ₂, —CH₂CO₂Z, —(CH₂)₂CO₂Z, —(CH₂)₄NZ₂,

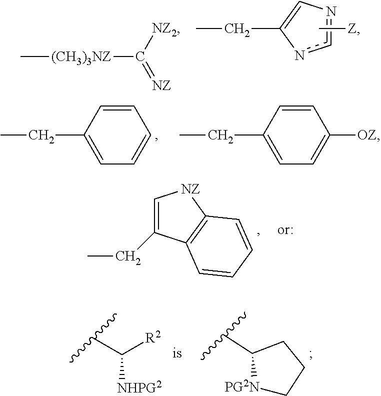

each Z is independently H, (C₁-C₃)alkyl, or a suitable protecting group; and
PG² is a suitable amine protecting group,
   under amide forming conditions to form the compound of formula 18 or a salt thereof.

In some embodiments, the amide forming conditions to form the compound of formula 18 comprise an amide coupling reagent selected from HATU, PyBOP, DCC, DIC, EDC, HBTU, HCTU, PyAOP, PyBrOP, BOP, BOP-Cl, DEPBT, T₃P, TATU, TBTU, TNTU, TOTU, TPTU, TSTU, and TDBTU. Without limitation, also provided herein is a process to prepare a compound of formula 18 by coupling a compound of formula 17 with a compound of formula 16 as described herein.

In some embodiments, the process for preparing a compound of formula 21 or formula 22 further comprises preparing the compound of formula 17, the process comprising removing the protecting group from the secondary amine of a compound of formula 15

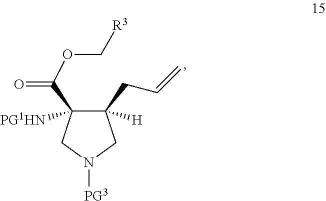

or a salt thereof, wherein:
R³ is a substituted or unsubstituted ring selected from aryl and heteroaryl; and
PG¹ and PG³ are independently suitable amine protecting groups,
   under suitable conditions to remove the protecting group from the secondary amine of the compound of formula 15 to form the compound of formula 17 or a salt thereof.

In some embodiments, the suitable conditions to remove the protecting group from the secondary amine of the compound of formula 15 comprise treating with a deprotecting reagent selected from acidic reagents, such as trifluoroacetic acid, tetra-N-buty 1 ammonium fluoride, hydrochloric acid, hydrobromic acid, phosphoric acid, sulfuric acid, methane sulfonic acid, p-toluene sulfonic acid, acetyl chloride, aluminum trichloride, and boron trifluoride. Without limitation, also provided herein is a process to prepare a compound of formula 17 from a compound of formula 15 as described herein.

In some embodiments, the process for preparing a compound of formula 21 or formula 22 further comprises preparing the compound of formula 15, the process comprising adding a protecting group to the primary amine of a compound of formula 14

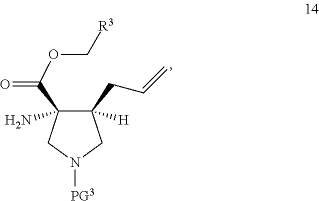

or a salt thereof, wherein:
R³ is a substituted or unsubstituted ring selected from aryl and heteroaryl; and
PG³ is a suitable amine protecting group,
   under suitable conditions to add the protecting group to the primary amine of the compound of formula 14 to form the compound of formula 15 or a salt thereof.

In some embodiments, the suitable conditions to add a protecting group to the primary amine of the compound of formula 14 comprise treating with benzyl chloroformate. Without limitation, also provided herein is a process to prepare a compound of formula 15 from a compound of formula 14 as described herein.

In some embodiments, the process for preparing a compound of formula 21 or formula 22 further comprises preparing the compound of formula 14, the process comprising reducing the azide of a compound of formula 13

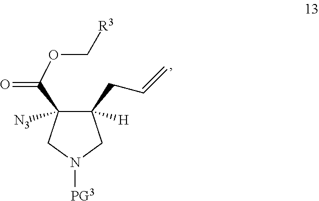

or a salt thereof, wherein:
R³ is a substituted or unsubstituted ring selected from aryl and heteroaryl; and
PG³ is a suitable amine protecting group,
   under suitable conditions to reduce the azide of a compound of formula 13 to form the compound of formula 14 or a salt thereof.

In some embodiments, the suitable conditions to reduce the azide of the compound of formula 13 comprise treating with zinc and acetic acid. Without limitation, also provided herein is a process to prepare a compound of formula 14 from a compound of formula 13 as described herein.

In some embodiments, the process for preparing a compound of formula 21 or formula 22 further comprises preparing the compound of formula 13, the process comprising adding a protecting group to the carboxylic acid of a compound of formula 12

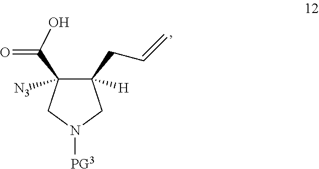

or a salt thereof, wherein:
PG³ is a suitable amine protecting group,
   under suitable conditions to add a protecting group to the carboxylic acid of a compound of formula 12 to form the compound of formula 13 or a salt thereof.

In some embodiments, the suitable conditions to add a protecting group to the carboxylic acid of the compound of formula 12 comprise treating with a benzyl halide and a base. Without limitation, also provided herein is a process to prepare a compound of formula 13 from a compound of formula 12 as described herein.

In some embodiments, the process for preparing a compound of formula 21 or formula 22 further comprises preparing the compound of formula 12 or formula 13, the process comprising treating a compound of formula 11

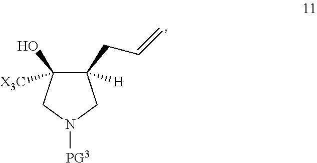

or a salt thereof, wherein:
each X is independently hydrogen or halogen; and
PG³ is a suitable amine protecting group,
   under suitable conditions to convert the compound of formula 11 into the compound of formula 12 or a salt thereof.

In some embodiments, the suitable conditions to convert the compound of formula 11 into the compound of formula 12 comprise treating with sodium or potassium azide and a base. Without limitation, also provided herein is a process to prepare a compound of formula 12 from a compound of formula 11 as described herein.

In some embodiments, the process for preparing a compound of formula 21 or formula 22 further comprises preparing the compound of formula 11, the process comprising removing the protecting group from the tertiary alcohol of a compound of formula 10

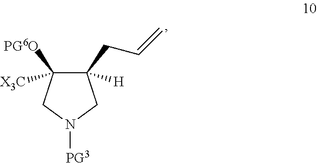

or a salt thereof, wherein:
each X is independently hydrogen or halogen;
PG³ is a suitable amine protecting group; and
PG⁶ is a suitable hydroxyl protecting group,
   under suitable conditions to remove the protecting group from the tertiary alcohol of the compound of formula 10 to form the compound of formula 11 or a salt thereof.

In some embodiments, the suitable conditions to deprotect the tertiary alcohol of the compound of formula 20 comprise treating with an acid or a fluoride source. Without limitation, also provided herein is a process to prepare a compound of formula 11 from a compound of formula 10 as described herein.

In some embodiments, the process for preparing a compound of formula 21 or formula 22 further comprises preparing the compound of formula 10 or formula 11, the process comprising treating a compound of formula 9

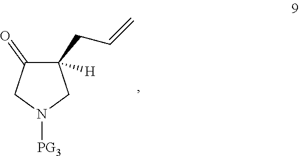

or a salt thereof, wherein:
PG³ is a suitable amine protecting group,
   under suitable conditions to alkylate the compound of formula 9 to form the compound of formula 10 or a salt thereof.

In some embodiments, the suitable conditions to alkylate the compound of formula 9 comprise treating with a trihalomethyltrimethylsilane or trichloromethyllithium. Without limitation, also provided herein is a process to prepare a compound of formula 10 or 11 from a compound of formula 9 as described herein.

In some embodiments, the process for preparing a compound of formula 21 or formula 22 further comprises preparing the compound of formula 9, the process comprising oxidizing the secondary alcohol of a compound of formula 8

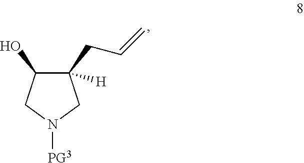

or a salt thereof, wherein:
PG³ is a suitable amine protecting group,
   under suitable conditions to oxidize the secondary alcohol of a compound of formula 8 to form the compound of formula 9 or a salt thereof.

In some embodiments, the suitable conditions to oxidize the secondary alcohol of a compound of formula 8 comprises treating with oxidizing conditions, such as with oxalyl chloride and DMSO. Without limitation, also provided herein is a process to prepare a compound of formula 9 from a compound of formula 8 as described herein.

In some embodiments, the process for preparing a compound of formula 21 or formula 22 further comprises preparing the compound of formula 8, the process comprising removing the protecting group from the secondary alcohol and the initial protecting group (i.e., PG^{N}, if PG^{N} is a suitable amine protecting group) from the secondary amine, and adding another protecting group to the secondary amine of a compound of formula 7

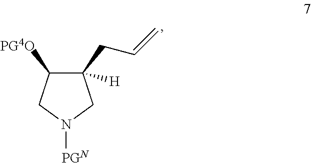

or a salt thereof, wherein:
PG⁴ is a suitable hydroxyl protecting group; and
PG^{N} is H or a suitable amine protecting group,
   under suitable conditions to remove the protecting group from the secondary alcohol and the initial protecting group (i.e., PG^{N}, if PG^{N} is a suitable amine protecting group) from the secondary amine, and adding another protecting group to the secondary amine of a compound of formula 7 to form the compound of formula 8 or a salt thereof.

In some embodiments, the suitable conditions to remove the protecting group from the secondary alcohol and the initial protecting group (i.e., PG^{N}, if PG^{N} is a suitable amine protecting group) from the secondary amine, and adding another protecting group to the secondary amine of the compound of formula 7 comprise treating with 1-chloroethyl chloroformate and di-tert-butyl dicarbonate. Without limitation, also provided herein is a process to prepare a compound of formula 8 from a compound of formula 7 as described herein.

In some embodiments, the process for preparing a compound of formula 21 or formula 22 further comprises preparing the compound of formula 7, the process comprising treating a compound of formula 6

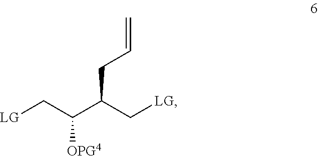

or a salt thereof, with H₂N-PG^{N}, wherein:
LG is a suitable leaving group;
PG⁴ is a suitable hydroxyl protecting group; and
PG^{N} is H or a suitable amine protecting group,
   under suitable conditions to perform a double displacement reaction on the compound of formula 6 to form the compound of formula 7 or a salt thereof.

In some embodiments, the suitable conditions to perform a double displacement reaction on the compound of formula 6 comprise H₂N-PG^{N} being benzylamine or ammonia. Without limitation, also provided herein is a process to prepare a compound of formula 7 from a compound of formula 6 as described herein.

In some embodiments, the process for preparing a compound of formula 21 or formula 22 further comprises preparing the compound of formula 6, the process comprising converting the primary alcohols of a compound of formula 5

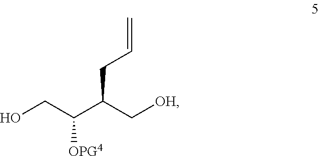

or a salt thereof, wherein:
PG⁴ is a suitable hydroxyl protecting group,
   into leaving groups under suitable conditions to form the compound of formula 6 or a salt thereof.

In some embodiments, the suitable conditions to convert the primary alcohols of the compound of formula 5 into leaving groups comprise treating with methanesulfonyl chloride, tosyl chloride, or triflic anhydride. Without limitation, also provided herein is a process to prepare a compound of formula 6 from a compound of formula 5 as described herein.

In some embodiments, the process for preparing a compound of formula 21 or formula 22 further comprises preparing the compound of formula 5, the process comprising reducing the alkyl carboxylates of a compound of formula 4

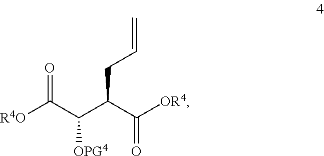

or a salt thereof, wherein:
each R⁴ is independently (C₁-C₆)alkyl; and
PG⁴ is a suitable hydroxyl protecting group,
   under suitable conditions to reduce the alkyl carboxylates of the compound of formula 4 to form the compound of formula 5 or a salt thereof.

In some embodiments, the suitable conditions to reduce the alkyl carboxylates of the compound of formula 4 comprise treating with a reducing agent, such as borane-dimethyl sulfide, sodium borohydride, lithium borohydride, potassium borohydride, lithium aluminum hydride, diisobutylaluminum hydride, or sodium bis(2-methoxyethoxy)aluminum hydride. Without limitation, also provided herein is a process to prepare a compound of formula 5 from a compound of formula 4 as described herein.

In some embodiments, the process for preparing a compound of formula 21 or formula 22 further comprises preparing the compound of formula 4, the process comprising adding a protecting group to the secondary alcohol of a compound of formula 3

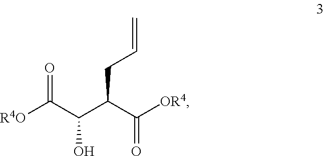

or a salt thereof, wherein:
each R⁴ is independently (C₁-C₆)alkyl;
   under suitable conditions to add the protecting group to the secondary alcohol of the compound of formula 3 to form the compound of formula 4 or a salt thereof.

In some embodiments, the suitable conditions to add a protecting group to the secondary alcohol of the compound of formula 3 comprise treating with 3,4-dihydropyran. Without limitation, also provided herein is a process to prepare a compound of formula 4 from a compound of formula 3 as described herein.

In some embodiments, the process for preparing a compound of formula 21 or formula 22 further comprises preparing the compound of formula 3, the process comprising allylating a compound of formula 2

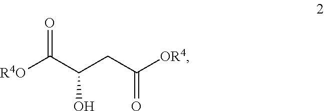

or a salt thereof, wherein:
each R⁴ is independently (C₁-C₃)alkyl;
   under suitable conditions to allylate the compound of formula 2 to form the compound of formula 3 or a salt thereof.

In some embodiments, the suitable conditions to allylate the compound of formula 2 comprise treating with an allyl electrophile, such as an allyl halide, such as allylbromide. Without limitation, also provided herein is a process to prepare a compound of formula 3 from a compound of formula 2 as described herein.

In some embodiments, the process for preparing a compound of formula 21 or formula 22 further comprises preparing the compound of formula 2, the process comprising esterifying L-malic acid under suitable conditions to form the compound of formula 2 or a salt thereof.

In some embodiments, the suitable conditions to esterify L-malic acid to form the compound of formula 2 comprises treating with acetyl chloride and a (C₁-C₃)alkyl alcohol, such as ethanol. Without limitation, also provided herein is a process to prepare a compound of formula 2 from L-malic acid as described herein.

In some embodiments of the process for preparing a compound of formula 21 or formula 22 provided herein, the suitable hydroxyl protecting group is selected from formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, methyl carbonate, 9-fluorenylmethyl carbonate, ethyl carbonate, 2,2,2-trichloroethyl carbonate, 2-(trimethylsilyl)ethyl carbonate, 2-(phenylsulfonyl)ethyl carbonate, vinyl carbonate, allyl carbonate, p-nitrobenzyl carbonate, trimethylsilyl, triethylsilyl, t-butyldimethylsilyl, t-butyldiphenylsilyl, triisopropylsilyl, methyl, ethyl, t-butyl, allyl, allyloxycarbonyl, methoxymethyl, methylthiomethyl, (2-methoxyethoxy)methyl, benzyloxymethyl, beta-(trimethylsilyl)ethoxymethyl, tetrahydropyranyl, benzyl, p-methoxybenzyl, 3,4-dimethoxybenzyl, O-nitrobenzyl, p-nitrobenzyl, p-halobenzyl, 2,6-dichlorobenzyl, p-cyanobenzyl, 2-picolyl, and 4-picolyl.

In some embodiments of the process for preparing a compound of formula 21 or formula 22 provided herein, the suitable amine protecting group is selected from tert-butyloxycarbonyl, ethyloxycarbonyl, methyloxycarbonyl, trichloroethyloxycarbonyl, allyloxycarbonyl, benzyloxocarbonyl, allyl, benzyl, fluorenylmethylcarbonyl, acetyl, chloroacetyl, dichloroacetyl, trichloroacetyl, trifluoroacetyl, phenylacetyl, and benzoyl.

In some embodiments of the process for preparing a compound of formula 21 or formula 22 provided herein, R¹ is ethyl or isopropyl.

In some embodiments of the process for preparing a compound of formula 21 or formula 22 provided herein, R² is H, methyl, or —CH₂OH.

In some embodiments of the process for preparing a compound of formula 21 or formula 22 provided herein, R³ is

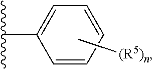

wherein each R is independently selected from halo, nitro, azido, cyano, aldehyde, amide, carboxylic acid, amino, hydroxyl, thiol, —(C₁-C₆)alkyl, —NH(C₁-C₆)alkyl, —N[(C₁-C₆)alkyl]₂, —CO(C₁-C₆)alkyl, —CO₂(C₁-C₆)alkyl, —O(C₁-C₆)alkyl, —S(C₁-C₆)alkyl, —NHCO(C₁-C₆)alkyl, and —NHCO₂(C₁-C₆)alkyl, aryl, and heteroaryl, and n is an integer from 0-5. In certain embodiments, R³ is phenyl.

In some embodiments of the process for preparing a compound of formula 21 or formula 22 provided herein, R³ is a heteroaryl ring substituted with (R⁵)ₙ, wherein each R⁵ is independently selected from halo, nitro, azido, cyano, aldehyde, amide, carboxylic acid, amino, hydroxyl, thiol, —(C₁-C₆)alkyl, —NH(C₁-C₆)alkyl, —N[(C₁-C₆)alkyl]₂, —CO(C₁-C₆)alkyl, —CO₂(C₁-C₆)alkyl, —O(C₁-C₆)alkyl, —S(C₁-C₆)alkyl, —NHCO(C₁-C₆)alkyl, and —NHCO₂(C₁-C₆)alkyl, aryl, and heteroaryl, and n is an integer from 0-5.

In some embodiments of the process for preparing a compound of formula 21 or formula 22 provided herein, R⁴ is selected from methyl, ethyl, and isopropyl.

In some embodiments of the process for preparing a compound of formula 21 or formula 22 provided herein, PG¹ is benzyloxocarbonyl. In some embodiments, PG² is benzyloxocarbonyl. In some embodiments, PG³ is tert-butyloxycarbonyl. In some embodiments, PG⁴ is tetrahydropyranyl. In some embodiments, PG^{N} is benzyl.

In some embodiments of the process for preparing a compound of formula 21 or formula 22 provided herein, each R is independently selected from methyl, ethyl, and isopropyl. In other embodiments of the process for preparing a compound of formula 21 or formula 22 provided herein, two R groups are taken together with their intervening atoms to form a substituted or unsubstituted monocyclic or bicyclic ring selected from

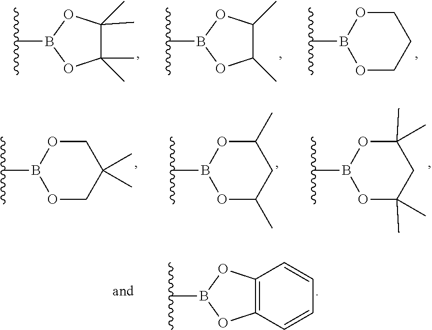

In some embodiments of the process for preparing a compound of formula 21 or formula 22 provided herein, LG is selected from halogen, mesylate, tosylate, and triflate.

In some embodiments of the process for preparing a compound of formula 21 or formula 22 provided herein, each X is independently selected from bromo and chloro.

Also provided herein is a compound of formula 20

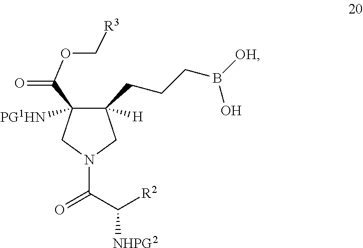

or a salt thereof, wherein:
R² is H, (C₁-C₆)alkyl, or —CH₂OZ; R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

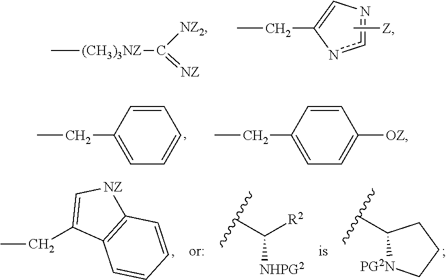

each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

In some embodiments, provided herein is a use of a compound of formula 20 to prepare a compound of formula 21 or formula 22, wherein variables R², R³, PG¹, and PG² are defined above and described herein.

Also provided herein is a compound of formula 19

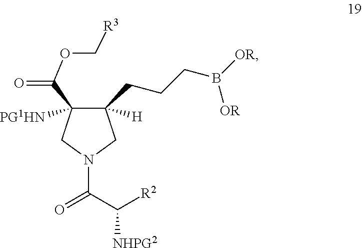

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

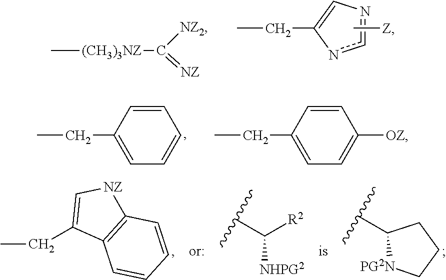

each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

In some embodiments, provided herein is a use of a compound of formula 19 to prepare a compound of formula 21 or formula 22, wherein variables R, R², R³, PG¹, and PG² are defined above and described herein.

Also provided herein is a compound of formula 18

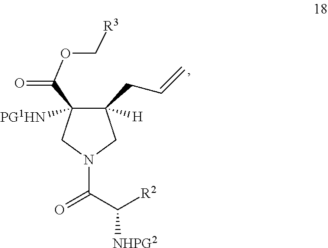

or a salt thereof, wherein:
R² is H, (C₁-C₆)alkyl, —CH₂OZ, —CH(CH₃)OZ, —CH₂SZ, ˜(CH₂)₂SCH₃, —CH₂CONZ₂, —(CH₂)₂CONZ₂, —CH₂CO₂Z, —(CH₂)₂CO₂Z, —(CH₂)₄NZ₂,

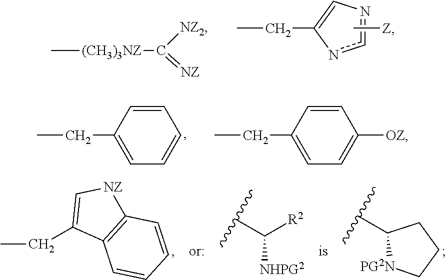

each Z is independently H, (C₁-C₃)alkyl, or a suitable protecting group;
R³ is a substituted or unsubstituted ring selected from aryl and heteroaryl; and
PG¹ and PG² are independently suitable amine protecting groups.

In some embodiments, provided herein is a use of a compound of formula 18 to prepare a compound of formula 21 or formula 22, wherein variables R², R³, PG¹, and PG² are defined above and described herein.

Also provided herein is a compound of formula 17

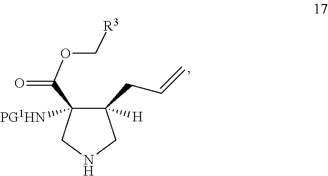

or a salt thereof, wherein:
R³ is a substituted or unsubstituted ring selected from aryl and heteroaryl; and
PG¹ is a suitable amine protecting group.

In some embodiments, provided herein is a use of a compound of formula 17 to prepare a compound of formula 21 or formula 22, wherein variables R³ and PG¹ are defined above and described herein.

Also provided herein is a compound of formula 15

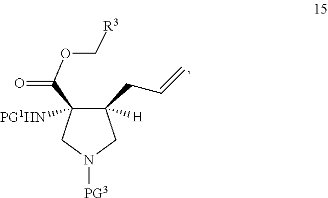

or a salt thereof, wherein:
R³ is a substituted or unsubstituted ring selected from aryl and heteroaryl; and
PG¹ and PG³ are independently suitable amine protecting groups.

In some embodiments, provided herein is a use of a compound of formula 15 to prepare a compound of formula 21 or formula 22, wherein variables R³, PG¹, and PG³ are defined above and described herein.

Also provided herein is a compound of formula 14

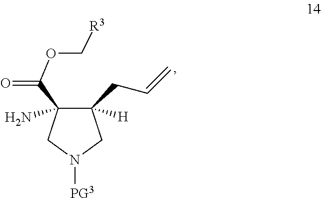

or a salt thereof, wherein:
R³ is a substituted or unsubstituted ring selected from aryl and heteroaryl; and
PG³ is a suitable amine protecting group.

In some embodiments, provided herein is a use of a compound of formula 14 to prepare a compound of formula 21 or formula 22, wherein variables R³ and PG³ are defined above and described herein.

Also provided herein is a compound of formula 13

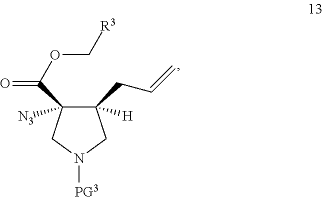

or a salt thereof, wherein:
R³ is a substituted or unsubstituted ring selected from aryl and heteroaryl; and
PG³ is a suitable amine protecting group.

In some embodiments, provided herein is a use of a compound of formula 13 to prepare a compound of formula 21 or formula 22, wherein variables R³ and PG³ are defined above and described herein.

In some embodiments, the compounds of the present disclosure have an enantiomeric excess of greater than 75% ee, greater than 80% ee, greater than 85% ee, greater than 90% ee, greater than 95% ee, greater than 96% ee, greater than 97% ee, greater than 98% ee, or even greater than 99% ee.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. In case of conflict, the present disclosure, including definitions, will control. Other features and advantages of the present disclosure will be apparent from the following detailed description and figures, and from the claims.

### 3. DETAILED DESCRIPTION

The methods and intermediate compounds of the present disclosure are useful for preparing the compounds of general formula 21 and formula 22, such as (3R,4S)-1-(L-alanyl)-3-amino-4-(3-boronopropyl)pyrrolidine-3-carboxylic acid, ((6aS,9aR)-9a-amino-8-((S)-2-aminopropanoyl)-3-ethoxyoctahydro-[1,2]oxaborocino[7,6-c]pyrrol-1(3H)-one), or analogs thereof including pharmaceutically acceptable salts thereof as described in U.S. Pat. Nos. 10,287,303, 10,287,303, and 10,494,339, the entireties of which are herein incorporated by reference. Starting materials are either commercially available or made by known procedures in the reported literature or as illustrated.

### 3.1. Definitions

As used herein, the following terms are intended to have the following meanings:

The terms “a” and “an” are used in this disclosure to refer to one or more than one (i.e., to at least one) of the grammatical object of the article.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10, more preferably from 1 to about 6 unless otherwise defined. Examples of straight chained and branched alkyl groups include methyl, ethyl, n-propyl, iso-propyl, n-butyl, sec-butyl, tert-butyl, pentyl, hexyl, pentyl, and octyl. A C₁-C₆ straight chained or branched alkyl group is also referred to as a “lower alkyl” group.

Moreover, the term “alkyl” (or “lower alkyl”) as used throughout the present disclosure is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, an alkoxy, a cyano, a nitro, an azido, a sulfhydryl, an alkylthio, a heterocyclyl, an aralkyl, or an aromatic or heteroaromatic moiety. In particular embodiments, the substituents on substituted alkyls are selected from C₁₋₆ alkyl, C₃₋₆ cycloalkyl, halogen, cyano, or hydroxyl. In more particular embodiments, the substituents on substituted alkyls are selected from fluoro, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of azido, imino, as well as ethers, alkylthios, —CF₃, —CN and the like. Exemplary substituted alkyls are described below. Cycloalkyls can be further substituted with alkyls, alkenyls, alkoxys, alkylthios, —CF₃, —CN, and the like.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

As used herein “amide coupling reagent” refers to a chemical reagent that facilitates the formation of a peptide bond (i.e., amide) through the reaction between typically a carboxylic acid and an amine. Exemplary amide coupling reagents include hexafluorophosphate azabenzotriazole tetramethyl uronium (“HATU”), benzotriazol-1-yl-oxytripyrrolidinophosphonium hexafluorophosphate (“PyBOP”), N,N′-dicyclohexylcarbodiimide (“DCC”), N,N′-diisopropylcarbodiimide (“DIC”), 1-ethyl-3-(3-dimethylaminopropyl)carbodiimide (“EDC”), 2-(1H-benzotriazol-1-yl)-1,1,3,3-tetramethyluronium hexafluorophosphate (“HBTU”), 0-(1H-6-chlorobenzotriazole-1-yl)-1,1,3,3-tetramethyluronium hexafluorophosphate (“HCTU”), (7-azabenzotriazol-1-yloxy)tripyrrolidinophosphonium hexafluorophosphate (“PyAOP”), bromo-tris-pyrrolidino-phosphonium hexafluorophosphate (“PyBrOP”), benzotriazol-1-yloxytris(dimethylamino)phosphonium hexafluorophosphate (“BOP”), bis(2-oxo-3-oxazolidinyl)phosphinic chloride (“BOP-Cl”), 3-(diethoxyphosphoryloxy)-1,2,3-benzotriazin-4(3H)-one (“DEPBT”), 1-propanephosphonic anhydride (“T₃P”), 0-(7-azabenzotriazole-1-yl)-N,N,N′,N′-tetramethyluronium tetrafluoroborate (“TATU”), 2-(1H-benzotriazole-1-yl)-1,1,3,3-tetramethylaminium tetrafluoroborate (“TBTU”), 2-(5-norbornene-2,3-dicarboximido)-1,1,3,3-tetramethyluronium tetrafluoroborate (“TNTU”), O-[(ethoxycarbonyl)cyanomethyleneamino]-N,N,N′,N′-tetra methyluronium tetrafluoroborate (“TOTU”), [dimethylamino-(2-oxopyridin-1-yl)oxymethylidene]-dimethylazanium; tetrafluoroborate (“TPTU”), N,N,N′,N′-Tetramethyl-O—(N-succinimidyl)uronium tetrafluoroborate (“TSTU”), and 2-(1H-benzotriazole-1-yl)-1,1,3,3-tetramethylaminium tetrafluoroborate (“TDBTU”).

As used herein, “crude form” refers to the physical state of a compound in that has not yet been treated with a purification step to remove residual impurities that may be present. As used herein, a “purified form” refers to the physical state of a compound that has been treated with a purification step (e.g., recrystallization) to remove impurities. The purity of a compound can be readily determined by known analytic techniques in the art (e.g., HPLC or UPLC). Generally, the purity of a purified form of a compound is greater than 90%, 95%, 96%, 97%, 98%, and even 99%. Based on the reaction conditions used to prepare a compound, a crude form of a compound can be used directly in subsequent steps without the need for a purification step.

The term “deprotecting agent” as used herein refers to a reagent or reagent system (reagent(s), and solvent) useful for removing a protecting group. Deprotecting agents can be acids, bases or reducing agents. For example, removal of the benzyl (Bn) group can be accomplished by reduction (hydrogenolysis), while removal of carbamates (e.g., Boc group) can be effected by use of acids (e.g., HCl, TFA, H₂SO₄, etc.), optionally with mild heating.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, boroxine, cyclic boronates, piperidine, piperazine, pyrrolidine, tetrahydropyran, tetrahydrofuran, morpholine, lactones, lactams, and the like.

As used herein, the phrase “leaving group” refers to a functional group that is displaced from a molecule during a chemical reaction. Leaving groups include halogens, as well sulfonate groups, such as tosylate, triflate, and mesylate.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

The term “protecting group” (“PG”) refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, N.Y. and Harrison et al., Compendium of Synthetic Organic Methods, Vols. 1-8, 1971-1996, John Wiley & Sons, N.Y. Representative nitrogen protecting groups include, but are not limited to, formyl, acetyl, trifluoroacetyl, benzyl, methoxymethyl (“MOM”), benzyloxycarbonyl (“CBZ”), tert-butoxycarbonyl (“Boc”), trimethylsilyl (“TMS”), 2-trimethylsilyl-ethanesulfonyl (“2-TES”), triethylsilyl (“TES”), triisopropylsilyl (“TIPS”), tert-butyldimethylsilyl (“TBDMS”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers. Representative carboxylic acid protecting groups include, but are not limited to, optionally substituted C₁₋₆ aliphatic esters, optionally substituted aryl esters, optionally substituted benzyl esters, silyl esters, dihydroxazoles, activated esters (e.g., derivatives of nitrophenol, pentafluorophenol, N-hydroxylsuccinimide, hydroxybenzotriazole, etc.), orthoesters, and the like.

As used herein, the phrase “reducing agent” refers generically to any species capable of reducing another species while itself being oxidized. As used herein, the phrase “oxidizing agent” or “oxidant” refers generically to any species capable of oxidizing another species while itself being reduced.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, an alkoxy, a cyano, a nitro, an azido, a sulfhydryl, an alkylthio, a heterocyclyl, an aralkyl, or an aromatic or heteroaromatic moiety. In particular embodiments, the substituents on substituted alkyls are selected from C₁₋₆ alkyl, C₃₋₆ cycloalkyl, halogen, cyano, or hydroxyl. In more particular embodiments, the substituents on substituted alkyls are selected from fluoro, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

### 3.2. Process for the Preparation of a Compound of Formula 21 or Formula 22

Provided herein is a process for preparing a compound of formula 21:

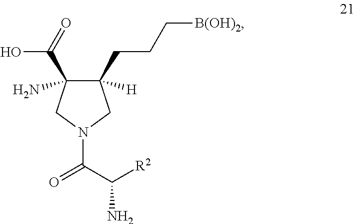

or a pharmaceutically acceptable salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

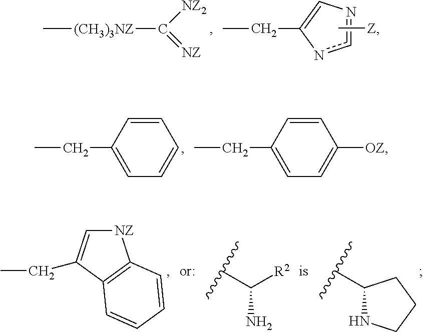

and
each Z is independently H, (C₁-C₆)alkyl, or a suitable protecting group.

In some embodiments, R² is H, (C₁-C₆)alkyl, or —CH₂OZ. In certain embodiments, R² is H. In some embodiments, R² is (C₁-C₆)alkyl. In some embodiments, R² is —CH₂OZ. In some embodiments, R² is methyl. In some embodiments, R² is —CH₂OH.

In one embodiment, the present methods provide a process of preparing a compound of formula 21, wherein the compound is:

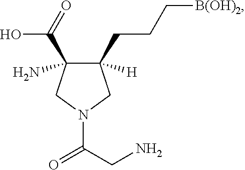 [SMILES: NCC(=O)N1C[C@H](CCCB(O)O)[C@](N)(C(=O)O)C1]

(3R,4S)-3-amino-4-(3-boronopropyl)-1-glycylpyrrolidine-3-carboxylic acid or a pharmaceutically acceptable salt thereof.

In one embodiment, the present methods provide a process of preparing a compound of formula 21, wherein the compound is:

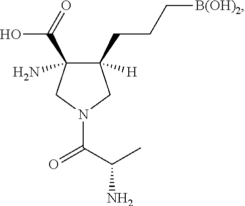 [SMILES: C[C@H](N)C(=O)N1C[C@H](CCCB(O)O)[C@](N)(C(=O)O)C1]

(3R,4S)-1-(L-alanyl)-3-amino-4-(3-boronopropyl)pyrrolidine-3-carboxylic acid or a pharmaceutically acceptable salt thereof.

In one embodiment, the present methods provide a process of preparing a compound of formula 21, wherein the compound is:

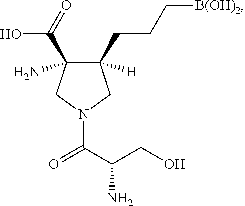 [SMILES: N[C@@H](CO)C(=O)N1C[C@H](CCCB(O)O)[C@](N)(C(=O)O)C1]

(3R,4S)-1-(L-seryl)-3-amino-4-(3-boronopropyl)pyrrolidine-3-carboxylic acid or a pharmaceutically acceptable salt thereof.

In other embodiments, the present methods provide a process of preparing a compound of formula 21′:

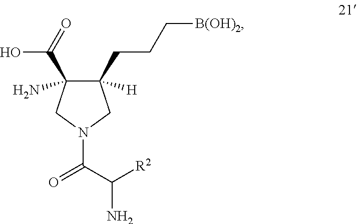

or a pharmaceutically acceptable salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

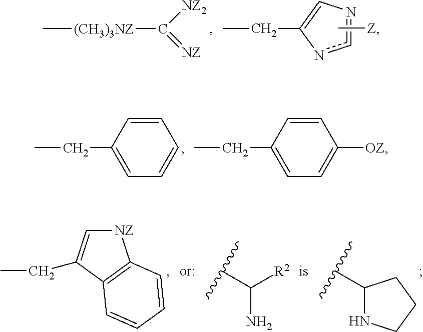

and
each Z is independently H, (C₁-C₃)alkyl, or a suitable protecting group.

Also provided herein is a process for preparing a compound of formula 22:

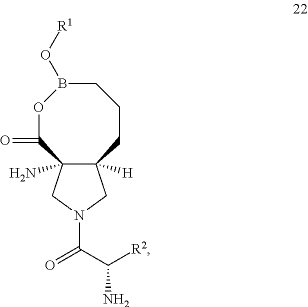

or a pharmaceutically acceptable salt thereof, wherein:
R¹ is H or (C₁-C₃)alkyl;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

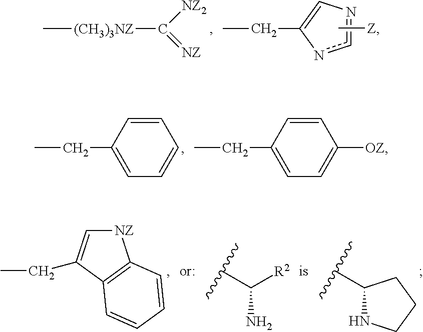

and
each Z is independently H, (C₁-C₃)alkyl, or a suitable protecting group.

In one embodiment, the present methods provide a process of preparing a compound of formula 22, wherein the compound is:

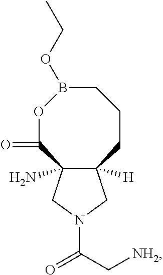 [SMILES: CCOB1CCC[C@H]2CN(C(=O)CN)C[C@@]2(N)C(=O)O1]

(6aS,9aR)-9a-amino-3-ethoxy-8-glycyloctahydro-[1,2]oxaborocino[6,7-c]pyrrol-1(3H)-one or a pharmaceutically acceptable salt thereof.

In another embodiment, the present methods provide a process of preparing a compound of formula 22, wherein the compound is:

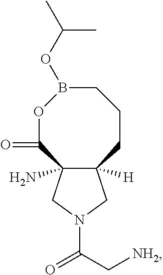 [SMILES: CC(C)OB1CCC[C@H]2CN(C(=O)CN)C[C@@]2(N)C(=O)O1]

(6aS,9aR)-9a-amino-3-iso-propoxy-8-glycyloctahydro-[1,2]oxaborocino[6,7-c]pyrrol-1(3H)-one or a pharmaceutically acceptable salt thereof.

In one embodiment, the present methods provide a process of preparing a compound of formula 22, wherein the compound is:

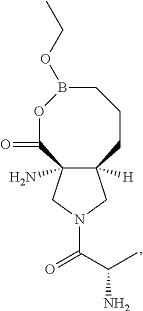 [SMILES: CCOB1CCC[C@H]2CN(C(=O)[C@H](C)N)C[C@@]2(N)C(=O)O1]

(6aS,9aR)-9a-amino-8-((S)-2-aminopropanoyl)-3-ethoxyoctahydro-[1,2]oxaborocino[7,6-c]pyrrol-1(3H)-one or a pharmaceutically acceptable salt thereof.

In one embodiment, the present methods provide a process of preparing a compound of formula 22, wherein the compound is:

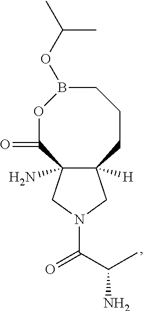 [SMILES: CC(C)OB1CCC[C@H]2CN(C(=O)[C@H](C)N)C[C@@]2(N)C(=O)O1]

(6aS,9aR)-9a-amino-8-((S)-2-aminopropanoyl)-3 iso-propoxyoctahydro-[1,2]oxaborocino[7,6-c]pyrrol-1(3H)-one or a pharmaceutically acceptable salt thereof.

In one embodiment, the present methods provide a process of preparing a compound of formula 22, wherein the compound is:

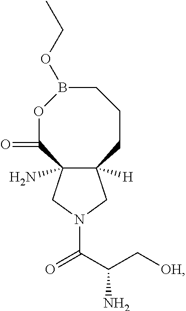 [SMILES: CCOB1CCC[C@H]2CN(C(=O)[C@@H](N)CO)C[C@@]2(N)C(=O)O1]

(6aS,9aR)-8-(L-seryl)-9a-amino-3-ethoxyoctahydro-[1,2]oxaborocino[6,7-c]pyrrol-1(3H)-one or a pharmaceutically acceptable salt thereof.

In one embodiment, the present methods provide a process of preparing a compound of formula 22, wherein the compound is:

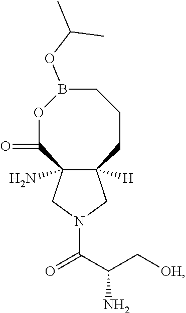 [SMILES: CC(C)OB1CCC[C@H]2CN(C(=O)[C@@H](N)CO)C[C@@]2(N)C(=O)O1]

(6aS,9aR)-8-(L-seryl)-9a-amino-3-iso-propoxyoctahydro-[1,2]oxaborocino[6,7-c]pyrrol-1(3H)-one or a pharmaceutically acceptable salt thereof.

In other embodiments, the present methods provide a process of preparing a compound of formula 22′:

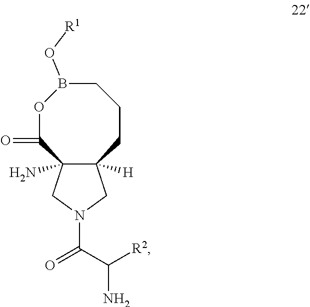

or a pharmaceutically acceptable salt thereof, wherein:
R¹ is H or (C₁-C₆)alkyl;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

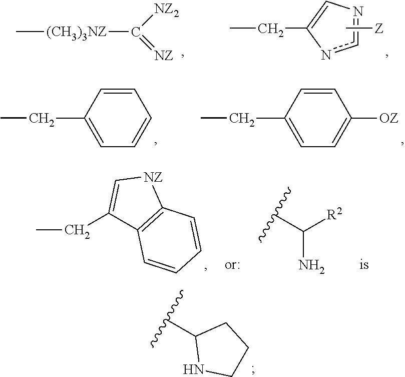

and
each Z is independently H, (C₁-C₆)alkyl, or a suitable protecting group.

The schemes provided herein are merely illustrative of some methods by which the compounds of the present disclosure can be synthesized, and various modifications of these schemes can be made and suggested by those skilled in the art having referred to this disclosure.

In some embodiments, a compound of formula 21 or formula 22 are prepared according to Scheme 1. Similarly, in certain embodiments, a compound of formula 21′ or formula 22′ are prepared according to Scheme 1, wherein compounds of formulas 16′, 18′, 19′, and 20′ are used in place of compounds of formulas 16, 18, 19, and 20, respectively.

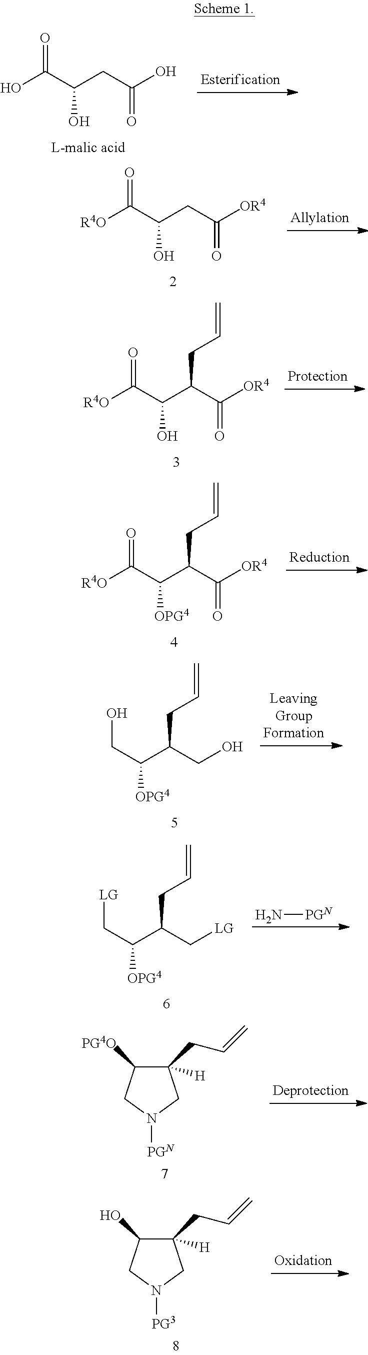

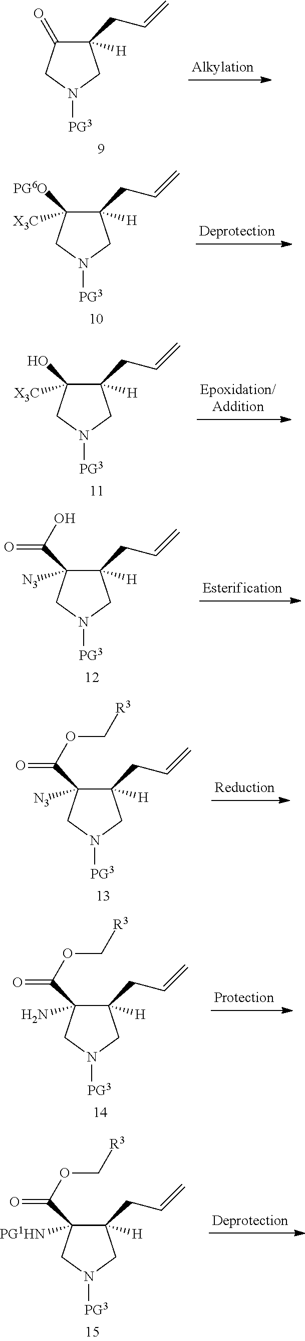

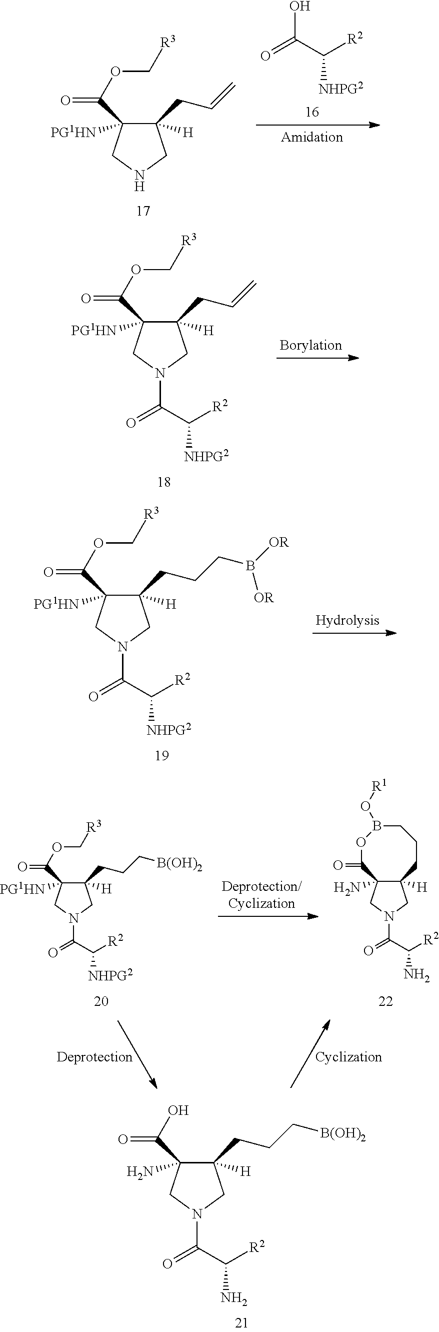

In Scheme 1 above, the reactions steps and variables LG, R, R¹, R², R³, R⁴, PG¹, PG², PG³, PG⁴, PG⁶, PG^{N}, and X are defined below and described herein.

In some embodiments, the process of preparing a compound of formula 21 or a pharmaceutically acceptable salt thereof comprises treating a compound of formula 20

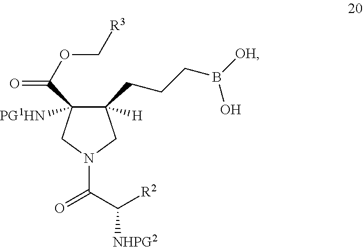

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

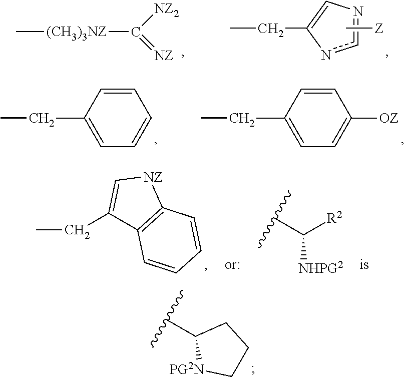

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to deprotect the compound of formula 20 to form the compound of formula 21 or a pharmaceutically acceptable salt thereof.

As defined above and described herein, R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

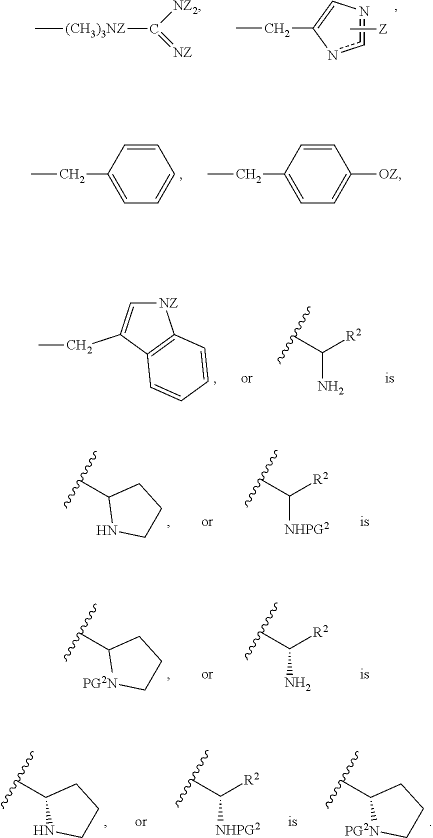

In some embodiments, R² is H, (C₁-C₃)alkyl, or —CH₂OZ. In certain embodiments, R² is H. In some embodiments, R² is (C₁-C₃)alkyl. In some embodiments, R² is —CH₂OZ. In some embodiments, R² is methyl. In some embodiments, R² is —CH₂OH. In some embodiments, R² is —CH(CH₃)OZ. In some embodiments, R² is —CH₂SZ. In some embodiments, R² is —(CH₂)₂SCH₃. In some embodiments, R² is —CH₂CONZ₂. In some embodiments, R² is —(CH₂)₂CONZ₂. In some embodiments, R² is —CH₂CO₂Z. In some embodiments, R² is —(CH₂)₂CO₂Z. In some embodiments, R² is —(CH₂)₄NZ₂. In some embodiments, R² is

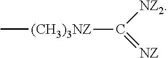

In some embodiments, R² is

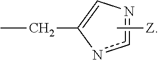

In some embodiments, R²

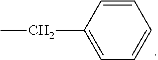

In some embodiments, R² is

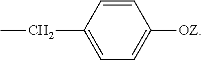

In some embodiments, R² is

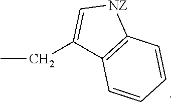

In some embodiments,

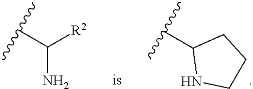

In some embodiments,

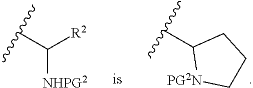

In some embodiments,

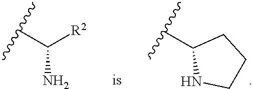

In some embodiments,

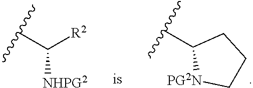

As defined above and described herein, each Z is independently H, (C₁-C₃)alkyl, or a suitable protecting group. In some embodiments, Z is H. In some embodiments, Z is (C₁-C₃)alkyl. In some embodiments, Z is a suitable protecting group. In some embodiments, Z is a suitable hydroxyl protecting group. In some embodiments, Z is a suitable amine protecting group. In some embodiments, Z is a suitable thiol protecting group. In some embodiments, Z is a suitable carboxylic acid protecting group.

Suitable hydroxyl protecting groups and the reagents and reaction conditions appropriate for using them to protect and deprotect hydroxyl groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. In certain embodiments, Z, taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, ethyl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl, 3,4-dimethoxybenzyl, O-nitrobenzyl, p-nitrobenzyl, p-halobenzyl, 2,6-dichlorobenzyl, p-cyanobenzyl, and 2- and 4-picolyl.

Suitable amine protecting groups and the reagents and reaction conditions appropriate for using them to protect and deprotect amine groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Without limitation, the amine protecting groups include protecting groups for the nitrogen atom(s) of amines, amides, imines, guanidines, imidazoles, and the like. Suitable amine protecting groups, taken with the nitrogen to which it is attached, include, but are not limited to, aralkylamines, carbamates, allyl amines, amides, and the like. Examples of PG¹ and PG² groups of the compounds of the formulae described herein include tert-butyloxycarbonyl (Boc), ethyloxycarbonyl, methyloxycarbonyl, trichloroethyloxycarbonyl, allyloxycarbonyl (Alloc), benzyloxocarbonyl (Cbz), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. In some embodiments, PG¹ is benzyloxocarbonyl (Cbz). In some embodiments, PG¹ is trifluoroacetyl. In some embodiments, PG² is benzyloxocarbonyl (Cbz).

Suitable carboxylate protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable carboxylate protecting groups include, but are not limited to, substituted C₁₋₆ aliphatic esters, optionally substituted aryl esters, silyl esters, activated esters (e.g., derivatives of nitrophenol, pentafluorophenol, N-hydroxylsuccinimide, hydroxybenzotriazole, etc.), orthoesters, and the like. Examples of such ester groups include methyl, ethyl, propyl, isopropyl, butyl, isobutyl, tert-butyl, benzyl, and phenyl wherein each group is optionally substituted.

Suitable thiol protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. As used herein, the phrase “suitable thiol protecting group” further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

As defined above and described herein, R³ is hydrogen or a substituted or unsubstituted ring selected from aryl and heteroaryl. In some embodiments, R³ is hydrogen. In some embodiments, R³ is a substituted or unsubstituted aryl. In some embodiments, R³ is a substituted or unsubstituted heteroaryl.

In some embodiments, R³ is (C₆-C₁₀)aryl or (C₅-C₉)heteroaryl. For example, in some instances R³ is

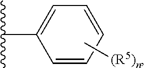

wherein:
each R⁵ is independently selected from halo, nitro, azido, cyano, aldehyde, amide, carboxylic acid, amino, hydroxyl, thiol, —(C₁-C₆)alkyl, —NH(C₁-C₆)alkyl, —N[(C₁-C₆)alkyl]₂—, —CO(C₁-C₆)alkyl, —CO₂(C₁-C₆)alkyl, —O(C₁-C₆)alkyl, —S(C₁-C₆)alkyl, —NHCO(C₁-C₆)alkyl, and —NHCO₂(C₁-C₆)alkyl, aryl, and heteroaryl; and
n is an integer from 0-5.

In certain embodiments, R³ is phenyl.

In some embodiments, R³ is a heteroaryl ring substituted with (R⁵)ₙ, wherein:
each R⁵ is independently selected from halo, nitro, azido, cyano, aldehyde, amide, carboxylic acid, amino, hydroxyl, thiol, —(C₁-C₃)alkyl, —NH(C₁-C₃)alkyl, —N[(C₁-C₃)alkyl]₂, —CO(C₁-C₃)alkyl, —CO₂(C₁-C₆)alkyl, —O(C₁-C₃)alkyl, —S(C₁-C₆)alkyl, —NHCO(C₁-C₆)alkyl, and —NHCO₂(C₁-C₆)alkyl, aryl, and heteroaryl; and
n is an integer from 0-5.

As defined above and described herein, PG¹ and PG² are independently suitable amine protecting groups.

In some embodiments, the suitable conditions used to prepare a compound of formula 21 from a compound of formula 20 include conditions known in the art to remove —CH₂R³, PG¹, and PG². In some embodiments, the suitable conditions include acidic conditions, basic conditions, reducing conditions, thermal conditions, or combinations thereof. In some embodiments, —CH₂R³, PG¹, and PG² are removed under acidic conditions (e.g., using mineral acids, organic acids, etc.). In some embodiments, —CH₂R³, PG¹, and PG² are removed under basic conditions (e.g., using hydroxides, organic bases, etc.). In some embodiments, —CH₂R³, PG¹, and PG² are removed under reducing conditions (e.g., using hydride reagents, hydrogenolysis with compositions comprising palladium, nickel, or platinum, etc.). In some embodiments, —CH₂R³, PG¹, and PG² are removed under thermal conditions. In certain embodiments, —CH₂R³, PG¹, and PG² are removed under reducing conditions (e.g., hydrogenolysis with a palladium composition).

In some embodiments, a process to prepare a compound of formula 21 from a compound of formula 20 includes providing a compound of formula 20 in a crude form or a purified form. In certain embodiments, a compound of formula 20 is provided in a crude form after one or more extraction, acidification, and solvent swapping steps.

In other embodiments, the process of preparing a compound of formula 21′ or a pharmaceutically acceptable salt thereof comprises treating a compound of formula 20′

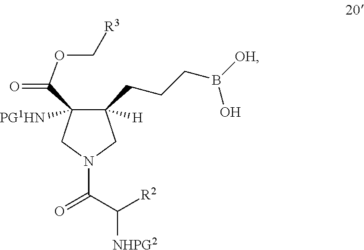

or a salt thereof, wherein:
R² is H, (C₁-C₆)alkyl, —CH₂OZ, —CH(CH₃)OZ, —CH₂SZ, ˜(CH₂)₂SCH₃, —CH₂CONZ₂, —(CH₂)₂CONZ₂, —CH₂CO₂Z, —(CH₂)₂CO₂Z, —(CH₂)₄NZ₂,

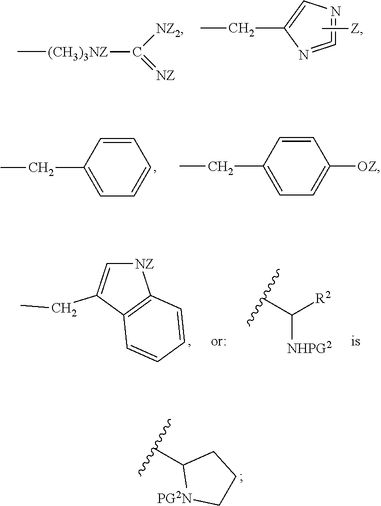

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to deprotect the compound of formula 20′ to form the compound of formula 21′ or a pharmaceutically acceptable salt thereof.

In some embodiments, the process of preparing a compound of formula 22 or a pharmaceutically acceptable salt thereof comprises treating a compound of formula 20

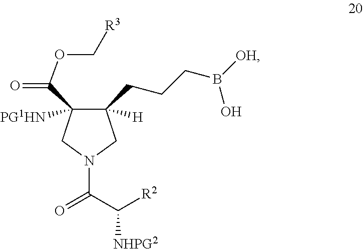

or a salt thereof, wherein:
R² is H, (C₁-C₆)alkyl, —CH₂OZ, —CH(CH₃)OZ, —CH₂SZ, ˜(CH₂)₂SCH₃, —CH₂CONZ₂, —(CH₂)₂CONZ₂, —CH₂CO₂Z, —(CH₂)₂CO₂Z, —(CH₂)₄NZ₂,

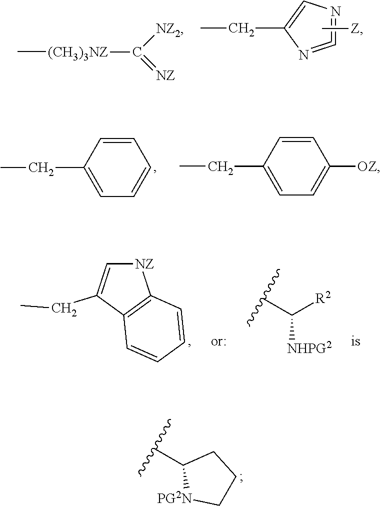

each Z is independently H, (C₁-C₃)alkyl, or a suitable protecting group;
R³ is a substituted or unsubstituted ring selected from aryl and heteroaryl; and
PG¹ and PG² are independently suitable amine protecting groups,
   under suitable conditions to deprotect and condense the compound of formula 20 to form the compound of formula 22 or a pharmaceutically acceptable salt thereof.

In some embodiments, the suitable conditions used to prepare a compound of formula 22 from a compound of formula 20 include conditions known in the art to remove —CH₂R³, PG¹, and PG² and promote intramolecular cyclization by a condensation reaction (e.g., removal of water). In some embodiments, the suitable conditions include acidic conditions, basic conditions, reducing conditions, thermal conditions, or combinations thereof. In some embodiments, —CH₂R³, PG¹, and PG² are removed under acidic conditions (e.g., using mineral acids, organic acids, etc.). In some embodiments, —CH₂R³, PG¹, and PG² are removed under basic conditions (e.g., using hydroxides, organic bases, etc.). In some embodiments, —CH₂R³, PG¹, and PG² are removed under reducing conditions (e.g., using hydride reagents, hydrogenolysis with compositions comprising palladium, nickel, or platinum, etc.). In some embodiments, —CH₂R³, PG¹, and PG² are removed under thermal conditions. In some embodiments, —CH₂R³, PG¹, and PG² are removed and intramolecular cyclization is promoted under the same conditions. In some embodiments, —CH₂R³, PG¹, and PG² are removed and intramolecular cyclization is promoted under different conditions. In some embodiments, intramolecular cyclization is promoted by azeotropic distillation. In some embodiments, intramolecular cyclization is promoted by slurrying in a hot alcohol.

In certain embodiments, —CH₂R³, PG¹, and PG² are removed under reducing conditions (e.g., hydrogenolysis with a palladium composition) and intramolecular cyclization is facilitated by azeotropic distillation in an alcohol (e.g., methanol, ethanol, isopropanol, and the like).

In some embodiments, a process to prepare a compound of formula 22 from a compound of formula 20 includes providing a compound of formula 20 in a crude form or a purified form. In certain embodiments, a compound of formula 20 is provided in a crude form after one or more extraction, acidification, and solvent swapping steps.

In certain embodiments, the process comprises preparing a compound of formula 21 or formula 22, wherein the compound of formula 20 is:

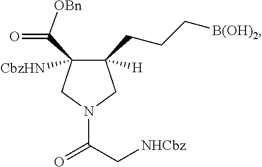 [SMILES: O=C(NCC(=O)N1C[C@H](CCCB(O)O)[C@](NC(=O)OCc2ccccc2)(C(=O)OCc2ccccc2)C1)OCc1ccccc1]

(3-((3 S,4R)-4-((benzyloxy)carbonyl)-4-(((benzyloxy)carbonyl)amino)-1-(((benzyloxy)carbonyl)glycyl)pyrrolidin-3-yl)propyl)boronic acid or salt thereof.

In certain embodiments, the process comprises preparing a compound of formula 21 or formula 22, wherein the compound of formula 20 is:

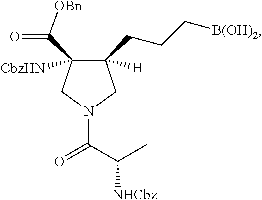 [SMILES: C[C@H](NC(=O)OCc1ccccc1)C(=O)N1C[C@H](CCCB(O)O)[C@](NC(=O)OCc2ccccc2)(C(=O)OCc2ccccc2)C1]

(3-((3S,4R)-4-((benzyloxy)carbonyl)-1-(((benzyloxy)carbonyl)-L-alanyl)-4-(((benzyloxy)carbonyl)amino)pyrrolidin-3-yl)propyl)boronic acid or salt thereof.

In certain embodiments, the process comprises preparing a compound of formula 21 or formula 22, wherein the compound of formula 20 is:

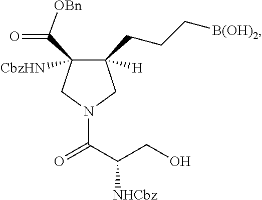 [SMILES: O=C(N[C@@H](CO)C(=O)N1C[C@H](CCCB(O)O)[C@](NC(=O)OCc2ccccc2)(C(=O)OCc2ccccc2)C1)OCc1ccccc1]

(3-((3S,4R)-4-((benzyloxy)carbonyl)-1-(((benzyloxy)carbonyl)-L-seryl)-4-(((benzyloxy)carbonyl)amino)pyrrolidin-3-yl)propyl)boronic acid or salt thereof.

In other embodiments, the process of preparing a compound of formula 21 or formula 22 a pharmaceutically acceptable salt thereof comprises using, in place of a compound of formula 20, a compound of formula 20*

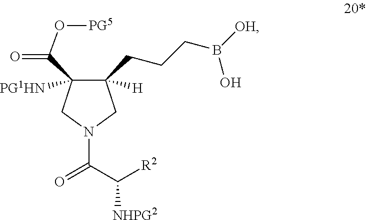

or a salt thereof, wherein:
R² is H, (C₁-C₆)alkyl, —CH₂OZ, —CH(CH₃)OZ, —CH₂SZ, ˜(CH₂)₂SCH₃, —CH₂CONZ₂, —(CH₂)₂CONZ₂, —CH₂CO₂Z, —(CH₂)₂CO₂Z, —(CH₂)₄NZ₂,

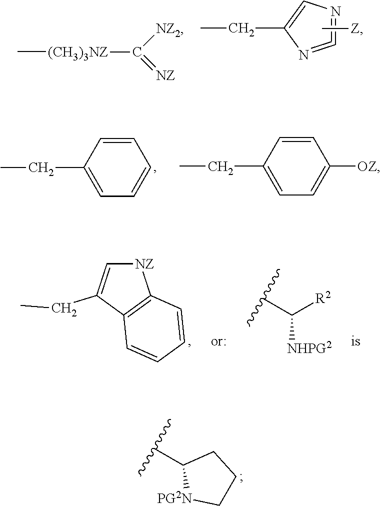

each Z is independently H, (C₁-C₆)alkyl, or a suitable protecting group;
PG¹ and PG² are independently suitable amine protecting groups,
PG⁵ is a suitable carboxylic acid protecting group.

In other embodiments, the process of preparing a compound of formula 22′ or a pharmaceutically acceptable salt thereof comprises treating a compound of formula 20′

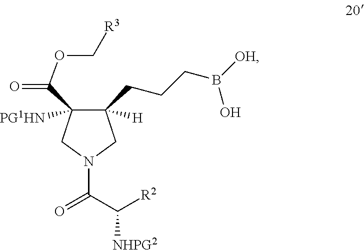

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

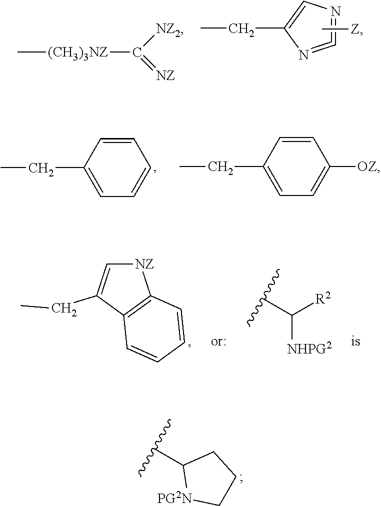

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to deprotect and condense the compound of formula 20′ to form the compound of formula 22′ or a pharmaceutically acceptable salt thereof.

In some embodiments, the process of preparing a compound of formula 22 or a pharmaceutically acceptable salt thereof comprises treating a compound of formula 21

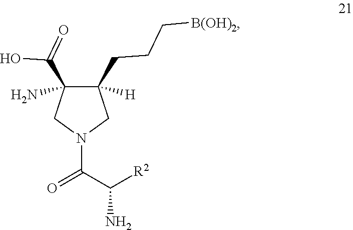

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

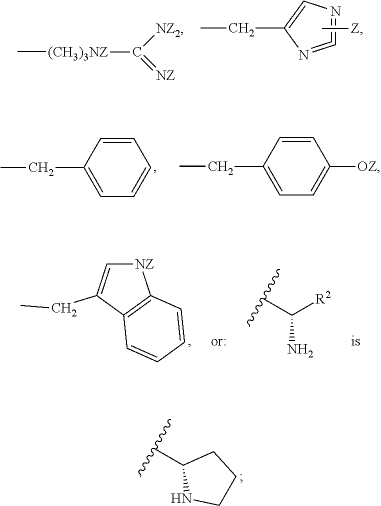

and
each Z is independently H, (C₁-C₆)alkyl, or a suitable protecting group,
   under suitable conditions to condense the compound of formula 21 to form the compound of formula 22 or a pharmaceutically acceptable salt thereof.

In some embodiments, the suitable conditions used to prepare a compound of formula 22 from a compound of formula 20 include conditions known in the art to promote intramolecular cyclization by a condensation reaction (e.g., removal of water). In some embodiments, the suitable conditions include acidic conditions, thermal conditions, condensation conditions, or combinations thereof. In some embodiments, intramolecular cyclization is promoted by azeotropic distillation. In some embodiments, intramolecular cyclization is promoted by slurrying in a hot alcohol.

In certain embodiments, intramolecular cyclization is facilitated by azeotropic distillation in an alcohol (e.g., methanol, ethanol, isopropanol, and the like).

In some embodiments, a process to prepare a compound of formula 22 from a compound of formula 20 includes providing a compound of formula 21 in a crude form or a purified form. In certain embodiments, a compound of formula 21 is provided in a crude form after one or more extraction, acidification, and solvent swapping steps.

In certain embodiments, the process comprises preparing a compound of formula 22, wherein the compound of formula 21 is:

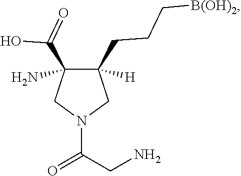 [SMILES: NCC(=O)N1C[C@H](CCCB(O)O)[C@](N)(C(=O)O)C1]

(3R,4S)-3-amino-4-(3-boronopropyl)-1-glycylpyrrolidine-3-carboxylic acid or salt thereof.

In certain embodiments, the process comprises preparing a compound of formula 22, wherein the compound of formula 21 is:

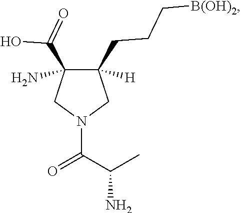 [SMILES: C[C@H](N)C(=O)N1C[C@H](CCCB(O)O)[C@](N)(C(=O)O)C1]

(3R,4S)-1-(L-alanyl)-3-amino-4-(3-boronopropyl)pyrrolidine-3-carboxylic acid or salt thereof.

In certain embodiments, the process comprises preparing a compound of formula 22, wherein the compound of formula 21 is:

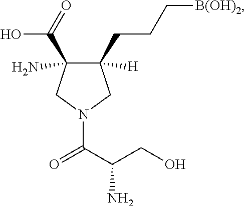 [SMILES: N[C@@H](CO)C(=O)N1C[C@H](CCCB(O)O)[C@](N)(C(=O)O)C1]

(3R,4S)-1-(L-seryl)-3-amino-4-(3-boronopropyl)pyrrolidine-3-carboxylic acid or salt thereof.

In other embodiments, the process of preparing a compound of formula 22′ or a pharmaceutically acceptable salt thereof comprises treating a compound of formula 21′

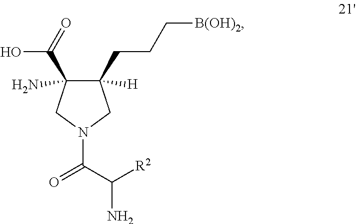

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

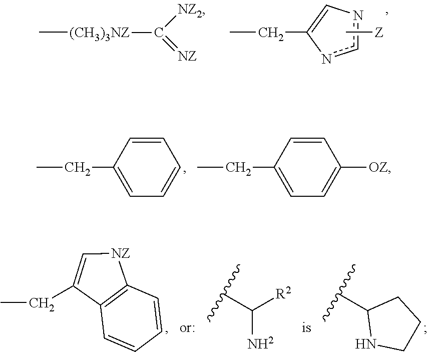

and
each Z is independently H, (C₁-C₆)alkyl, or a suitable protecting group,
   under suitable conditions to condense the compound of formula 21′ to form the compound of formula 22′ or a pharmaceutically acceptable salt thereof.

In some embodiments, the process for preparing a compound of formula 21 or formula 22 further comprises preparing the compound of formula 20, the process comprising hydrolyzing the boronate of a compound of formula 19

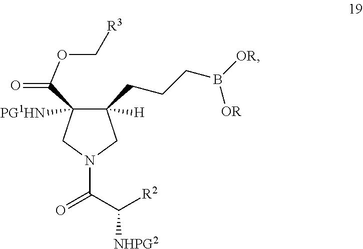

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

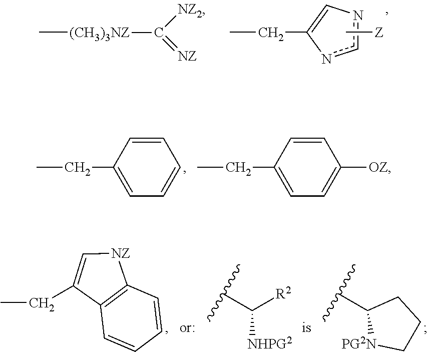

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to hydrolyze the boronate of the compound of formula 19 to form the compound of formula 20 or a salt thereof.

As defined above and described herein, each R is independently (C₁-C₆)alkyl, or two R groups are optionally taken together with their intervening atoms to form a substituted or unsubstituted monocyclic or bicyclic ring selected from a saturated or partially unsaturated heterocycle.

In some embodiments, each R is (C₁-C₆)alkyl. In some embodiments, each R is methyl. In some embodiments, each R is ethyl. In some embodiments, each R is isopropyl. In some embodiments, two R groups are optionally taken together with their intervening atoms to form a substituted or unsubstituted monocyclic or bicyclic ring selected from a saturated or partially unsaturated heterocycle. In some embodiments, two R groups form

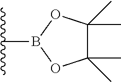 [SMILES: *B1OC(C)(C)C(C)(C)O1]

(i.e., pinacol boronate). In some embodiments, two R groups form

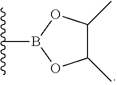 [SMILES: *B1OC(C)C(C)O1]

In some embodiments, two R groups form

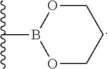 [SMILES: *B1OCCCO1]

In some embodiments, two R groups form

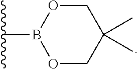 [SMILES: *B1OCC(C)(C)CO1]

In some embodiments, two R groups form

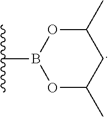 [SMILES: *B1OC(C)CC(C)O1]

In some embodiments, two R groups form

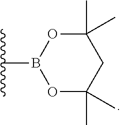 [SMILES: *B1OC(C)(C)CC(C)(C)O1]

In some embodiments, two R groups form

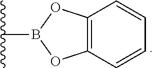 [SMILES: *B1Oc2ccccc2O1]

In some embodiments, the suitable conditions used to prepare a compound of formula 20 from a compound of formula 19 include conditions known in the art to hydrolyze a boronic esters to a boronic acid. In some embodiments, the suitable conditions include oxidative cleavage with sodium periodate, biphasic transesterification with other boronic acids, transborylation with boron trichloride, acidic hydrolysis, solid-phase processes using polystyrene-boronic acid, formation of fluorinated intermediates followed by trimethylsilyl chloride or lithium hydroxide, or transesterification with diethanolamine followed by acidic hydrolysis. In certain embodiments, the boronic ester is pinacol boronate and deprotection of the pinacol boronate of a compound of formula 19 is achieved using sodium periodate in an acidic medium.

In some embodiments, a process to prepare a compound of formula 20 from a compound of formula 19 includes providing a compound of formula 19 in a crude form or a purified form. In certain embodiments, a compound of formula 19 is provided in a purified form after one or more extraction, washing, solvent swapping, and crystallization steps. In some embodiments, a compound of formula 19 is isolated as a solid appropriate for use as an intermediate compound. For instance, in one embodiment, a compound of formula 19 is isolated as a solid appropriate for use in producing a compound of formula 21 or formula 22. In one such embodiment, a compound of formula 19 is a crystalline solid for use in producing a compound of formula 21 or formula 22.

In certain embodiments, the process comprises preparing a compound of formula 20, wherein the compound of formula 19 is:

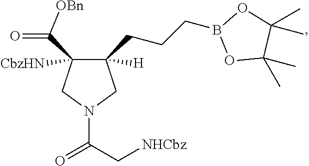 [SMILES: CC1(C)OB(CCC[C@H]2CN(C(=O)CNC(=O)OCc3ccccc3)C[C@@]2(NC(=O)OCc2ccccc2)C(=O)OCc2ccccc2)OC1(C)C]

benzyl (3R,4S)-3-(((benzyloxy)carbonyl)amino)-1-(((benzyloxy)carbonyl)glycyl)-4-(3-(4,4,5,5-tetramethyl-1,3,2-dioxaborolan-2-yl)propyl)pyrrolidine-3-carboxylate or salt thereof.

In certain embodiments, the process comprises preparing a compound of formula 20, wherein the compound of formula 19 is:

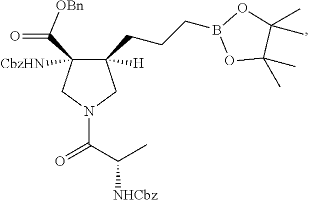 [SMILES: C[C@H](NC(=O)OCc1ccccc1)C(=O)N1C[C@H](CCCB2OC(C)(C)C(C)(C)O2)[C@](NC(=O)OCc2ccccc2)(C(=O)OCc2ccccc2)C1]

benzyl (3R,4S)-1-(((benzyloxy)carbonyl)-L-alanyl)-3-(((benzyloxy)carbonyl)amino)-4-(3-(4,4,5,5-tetramethyl-1,3,2-dioxaborolan-2-yl)propyl)pyrrolidine-3-carboxylate or salt thereof.

In certain embodiments, the process comprises preparing a compound of formula 20, wherein the compound of formula 19 is:

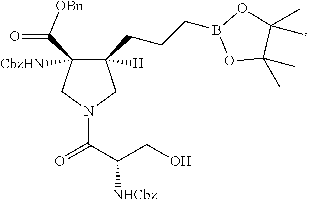 [SMILES: CC1(C)OB(CCC[C@H]2CN(C(=O)[C@H](CO)NC(=O)OCc3ccccc3)C[C@@]2(NC(=O)OCc2ccccc2)C(=O)OCc2ccccc2)OC1(C)C]

benzyl (3R,4S)-1-(((benzyloxy)carbonyl)-L-seryl)-3-(((benzyloxy)carbonyl)amino)-4-(3-(4,4,5,5-tetramethyl-1,3,2-dioxaborolan-2-yl)propyl)pyrrolidine-3-carboxylate or salt thereof.

In other embodiments, the process for preparing a compound of formula 21 or formula 22 comprises using, in place of a compound of formula 19, a compound of formula 19*

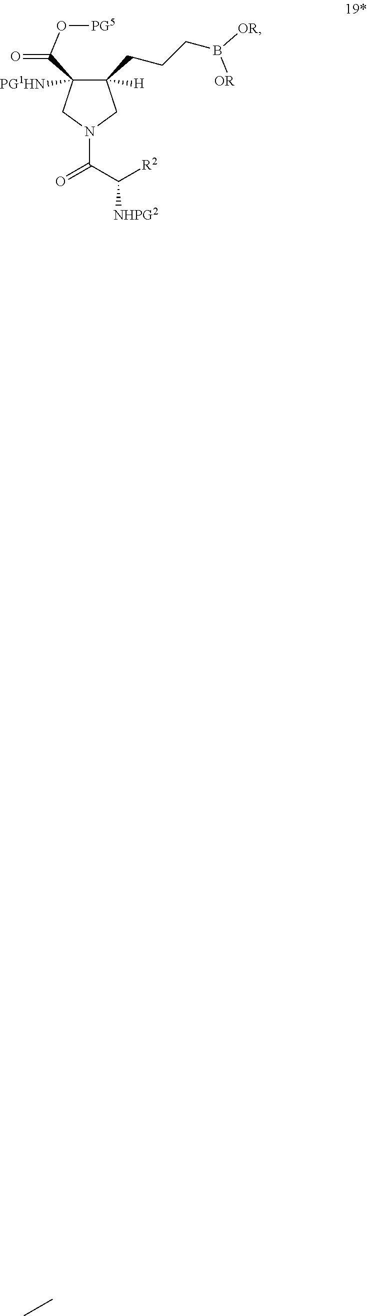

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

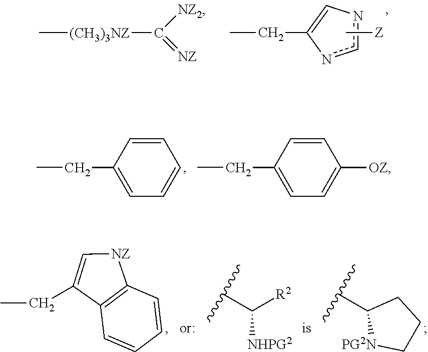

each Z is independently H, (C₁-C₆)alkyl, or a suitable protecting group;
PG¹ and PG² are independently suitable amine protecting groups; and
PG⁵ is a suitable carboxylic acid protecting group.

In other embodiments, the process for preparing a compound of formula 21′ or formula 22′ further comprises preparing the compound of formula 20′, the process comprising hydrolyzing the boronate of a compound of formula 19′

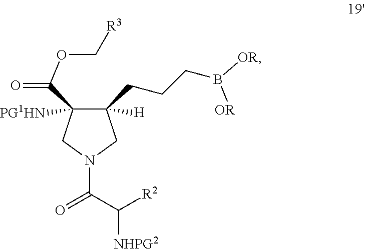

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

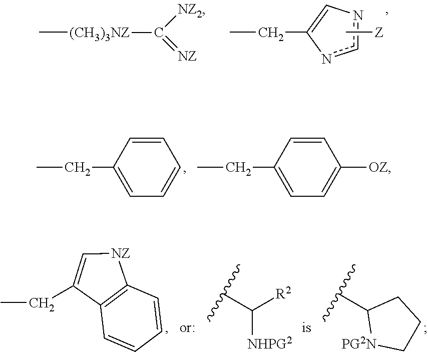

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to hydrolyze the boronate of the compound of formula 19′ to form the compound of formula 20′ or a salt thereof.

In some embodiments, the process for preparing a compound of formula 21 or formula 22 further comprises preparing the compound of formula 19, the process comprising hydroborating a compound of formula 18

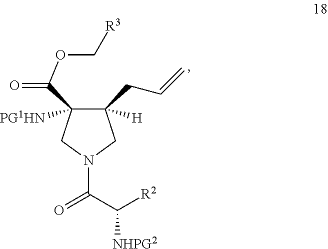

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

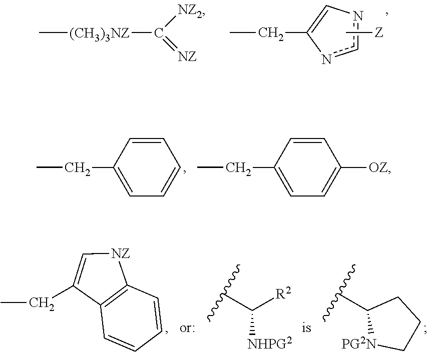

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to form the compound of formula 19 or a salt the under suitable conditions to hydroborate the compound of formula 18 to form the compound of formula 19 or a salt thereof.

In some embodiments, the suitable conditions used to prepare a compound of formula 19 from a compound of formula 18 include conditions known in the art to hydroborate an alkene, specifically a terminal alkene. In some embodiments, the suitable conditions include hydroboration using a borane reagent or metal-catalyzed hydroboration using catechol borane, pinacolborane, or bis(pinacolato)diboron (B₂Pin₂) and a metal catalyst of rhodium, iridium, iron, and the like. In certain embodiments, the suitable conditions include hydroboration using an iridium catalyst and pinacolborane.

In some embodiments, a process to prepare a compound of formula 19 from a compound of formula 18 includes providing a compound of formula 18 in a crude form or a purified form. In certain embodiments, a compound of formula 18 is provided in a purified form after one or more extraction, washing, solvent swapping, and crystallization steps. In some embodiments, a compound of formula 18 is isolated as a solid appropriate for use as an intermediate compound. For instance, in one embodiment, a compound of formula 18 is isolated as a solid appropriate for use in producing a compound of formula 21 or formula 22. In one such embodiment, a compound of formula 18 is a crystalline solid for use in producing a compound of formula 21 or formula 22.

In certain embodiments, the process comprises preparing a compound of formula 19, wherein the compound of formula 18 is:

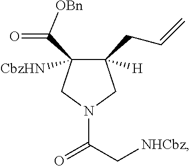 [SMILES: C=CC[C@H]1CN(C(=O)CNC(=O)OCc2ccccc2)C[C@@]1(NC(=O)OCc1ccccc1)C(=O)OCc1ccccc1]

benzyl (3R,4S)-4-allyl-3-(((benzyloxy)carbonyl)amino)-1-(((benzyloxy)carbonyl)glycyl)pyrrolidine-3-carboxylate or salt thereof.

In certain embodiments, the process comprises preparing a compound of formula 19, wherein the compound of formula 18 is:

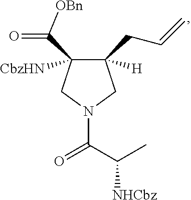 [SMILES: C=CC[C@H]1CN(C(=O)[C@H](C)NC(=O)OCc2ccccc2)C[C@@]1(NC(=O)OCc1ccccc1)C(=O)OCc1ccccc1]

benzyl (3R,4S)-4-allyl-1-(((benzyloxy)carbonyl)-L-alanyl)-3-(((benzyloxy)carbonyl)amino)pyrrolidine-3-carboxylate or salt thereof.

In certain embodiments, the process comprises preparing a compound of formula 19, wherein the compound of formula 18 is:

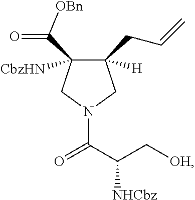 [SMILES: C=CC[C@H]1CN(C(=O)[C@H](CO)NC(=O)OCc2ccccc2)C[C@@]1(NC(=O)OCc1ccccc1)C(=O)OCc1ccccc1]

benzyl (3R,4S)-4-allyl-1-(((benzyloxy)carbonyl)-L-seryl)-3-(((benzyloxy)carbonyl)amino)pyrrolidine-3-carboxylate or salt thereof.

In certain embodiments, the process comprises preparing a compound of formula 19, wherein the compound of formula 18 is:

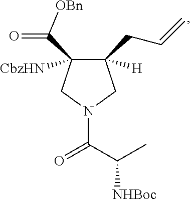 [SMILES: C=CC[C@H]1CN(C(=O)[C@H](C)NC(=O)OC(C)(C)C)C[C@@]1(NC(=O)OCc1ccccc1)C(=O)OCc1ccccc1]

benzyl (3R,4S)-4-allyl-3-(((benzyloxy)carbonyl)amino)-1-((tert-butoxycarbonyl)-L-alanyl)pyrrolidine-3-carboxylate or salt thereof.

In certain embodiments, the process comprises preparing a compound of formula 19, wherein the compound of formula 18 is:

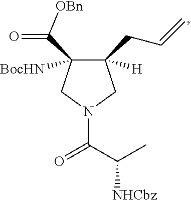 [SMILES: C=CC[C@H]1CN(C(=O)[C@H](C)NC(=O)OCc2ccccc2)C[C@@]1(NC(=O)OC(C)(C)C)C(=O)OCc1ccccc1]

benzyl (3R,4S)-4-allyl-1-(((benzyloxy)carbonyl)-L-alanyl)-3-((tert-butoxycarbonyl)amino)pyrrolidine-3-carboxylate or salt thereof.

In certain embodiments, the process comprises preparing a compound of formula 19, wherein the compound of formula 18 is:

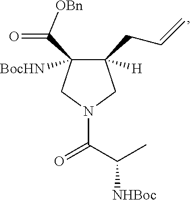 [SMILES: C=CC[C@H]1CN(C(=O)[C@H](C)NC(=O)OC(C)(C)C)C[C@@]1(NC(=O)OC(C)(C)C)C(=O)OCc1ccccc1]

benzyl (3R,4S)-4-allyl-1-((tert-butoxycarbonyl)-L-alanyl)-3-((tert-butoxycarbonyl)amino)pyrrolidine-3-carboxylate or salt thereof.

In certain embodiments, the process comprises preparing a compound of formula 19, wherein the compound of formula 18 is:

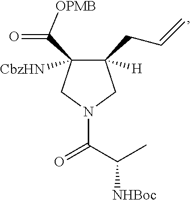

4-methoxybenzyl (3R,4S)-4-allyl-3-(((benzyloxy)carbonyl)amino)-1-((tert-butoxycarbonyl)-L-alanyl)pyrrolidine-3-carboxylate or salt thereof.

In certain embodiments, the process comprises preparing a compound of formula 19, wherein the compound of formula 18 is:

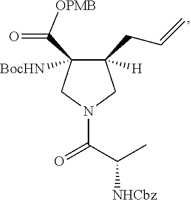

4-methoxybenzyl (3R,4S)-4-allyl-1-(((benzyloxy)carbonyl)-L-alanyl)-3-((tert-butoxycarbonyl)amino)pyrrolidine-3-carboxylate or salt thereof.

In certain embodiments, the process comprises preparing a compound of formula 19, wherein the compound of formula 18 is:

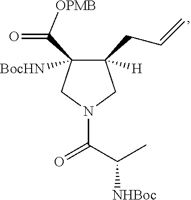

4-methoxybenzyl (3R,4S)-4-allyl-1-((tert-butoxycarbonyl)-L-alanyl)-3-((tert-butoxycarbonyl)amino)pyrrolidine-3-carboxylate or salt thereof.

In other embodiments, the process for preparing a compound of formula 21 or formula 22 comprises using, in place of a compound of formula 18, a compound of formula 18*

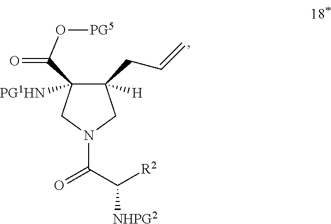

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

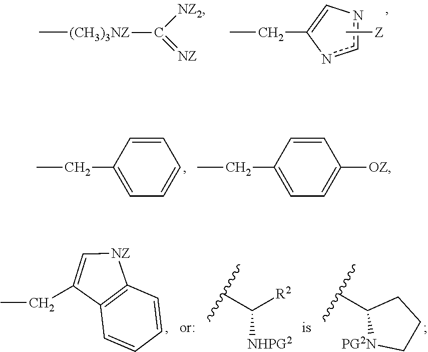

each Z is independently H, (C₁-C₆)alkyl, or a suitable protecting group;
PG and PG² are independently suitable amine protecting groups; and
PG⁵ is a suitable carboxylic acid protecting group.

In other embodiments, the process for preparing a compound of formula 21′ or formula 22′ further comprises preparing the compound of formula 19′, the process comprising hydroborating a compound of formula 18′

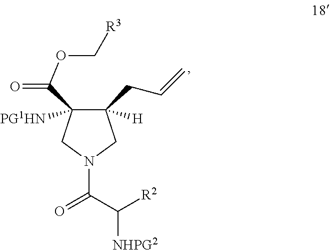

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

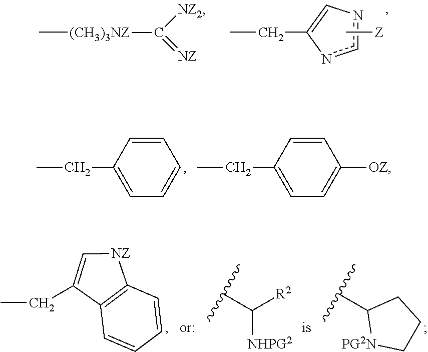

each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
   under suitable conditions to form the compound of formula 19′ or a salt the under suitable conditions to hydroborate the compound of formula 18′ to form the compound of formula 19′ or a salt thereof.

In some embodiments, the process for preparing a compound of formula 21 or formula 22 further comprises preparing the compound of formula 18, the process comprising coupling a compound of formula 17

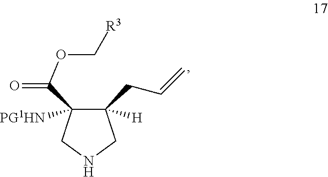

or a salt thereof, wherein:
R³ is a substituted or unsubstituted ring selected from aryl and heteroaryl; and
PG¹ is a suitable amine protecting group,
   with a compound of formula 16

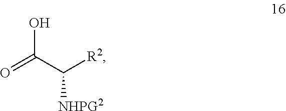

or salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

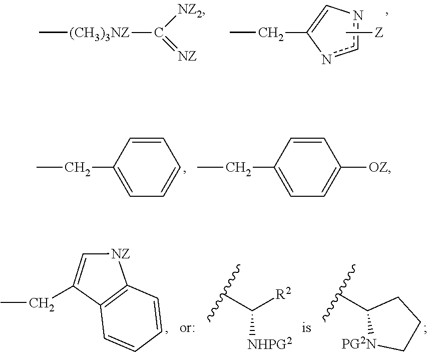

each Z is independently H, (C₁-C₆)alkyl, or a suitable protecting group; and
PG² is a suitable amine protecting group,
   under amide forming conditions to form the compound of formula 18 or a salt thereof.

In some embodiments, the amide forming conditions used to prepare a compound of formula 18 from a compound of formula 17 and a compound of formula 16 include conditions known in the art to form an amide bond between a secondary amine and a carboxylic acid. In some embodiments, the amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T₃P, TATU, TBTU, TNTU, TOTU, TPTU, TSTU, or TDBTU. In certain embodiments, the amide coupling reagent is 1-propanephosphonic anhydride solution (T₃P).

In some embodiments, a process to prepare a compound of formula 18 from a compound of formula 17 includes providing a compound of formula 17 in a crude form or a purified form. In certain embodiments, a compound of formula 17 is provided in a purified form after one or more extraction, washing, solvent swapping, and crystallization steps. In some embodiments, a compound of formula 17 is isolated as a solid appropriate for use as an intermediate compound. For instance, in one embodiment, a compound of formula 17 is isolated as a solid appropriate for use in producing a compound of formula 21 or formula 22. In one such embodiment, a compound of formula 17 is a crystalline solid for use in producing a compound of formula 21 or formula 22.

In certain embodiments, the process comprises preparing a compound of formula 18, wherein the compound of formula 17 is:

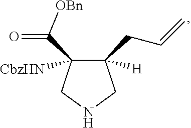 [SMILES: C=CC[C@H]1CNC[C@@]1(NC(=O)OCc1ccccc1)C(=O)OCc1ccccc1]

benzyl (3R,4S)-4-allyl-3-(((benzyloxy)carbonyl)amino)pyrrolidine-3-carboxylate or salt thereof.

In other embodiments, the process for preparing a compound of formula 21 or formula 22 comprises using, in place of a compound of formula 17, a compound of formula 17*

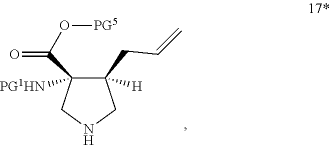

or a salt thereof, wherein:
PG¹ is a suitable amine protecting group; and
PG⁵ is a suitable carboxylic acid protecting group.

In certain embodiments, the process comprises preparing a compound of formula 18, wherein the compound of formula 16 is:

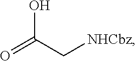 [SMILES: O=C(O)CNC(=O)OCc1ccccc1]

((benzyloxy)carbonyl)glycine or salt thereof.

In certain embodiments, the process comprises preparing a compound of formula 18, wherein the compound of formula 16 is:

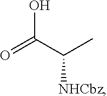 [SMILES: C[C@H](NC(=O)OCc1ccccc1)C(=O)O]

((benzyloxy)carbonyl)-L-alanine or salt thereof.

In certain embodiments, the process comprises preparing a compound of formula 18, wherein the compound of formula 16 is:

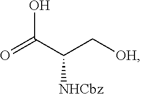 [SMILES: O=C(N[C@@H](CO)C(=O)O)OCc1ccccc1]

((benzyloxy)carbonyl)-L-serine or salt thereof.

In other embodiments, the process for preparing a compound of formula 21′ or formula 22′ further comprises preparing the compound of formula 18′, the process comprising coupling a compound of formula 17

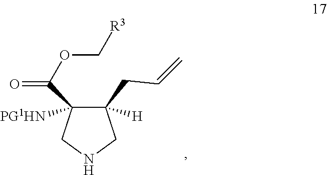

or a salt thereof, wherein:
R³ is a substituted or unsubstituted ring selected from aryl and heteroaryl; and
PG¹ is a suitable amine protecting group,
   with a compound of formula 16′

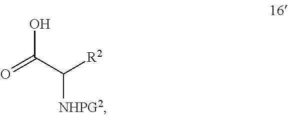

or salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

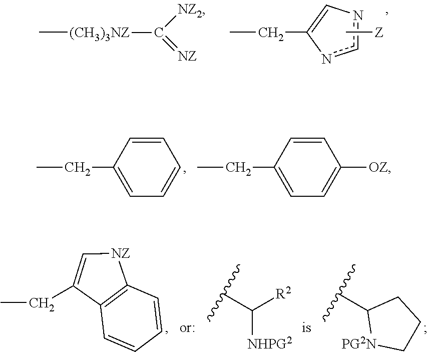

each Z is independently H, (C₁-C₆)alkyl, or a suitable protecting group; and
PG² is a suitable amine protecting group,
   under amide forming conditions to form the compound of formula 18′ or a salt thereof.

In some embodiments, the process for preparing a compound of formula 21 or formula 22 further comprises preparing the compound of formula 17, the process comprising removing the protecting group from the secondary amine of a compound of formula 15

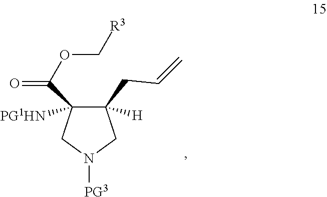

or a salt thereof, wherein:
R³ is a substituted or unsubstituted ring selected from aryl and heteroaryl; and
PG¹ and PG³ are independently suitable amine protecting groups,
   under suitable conditions to remove the protecting group from the secondary amine of the compound of formula 15 to form the compound of formula 17 or a salt thereof.

In some embodiments, the suitable conditions used to prepare a compound of formula 17 from a compound of formula 15 include conditions known in the art to remove a protecting group (e.g., PG³) from a secondary amine. In some embodiments, PG³ of the formulae described herein is selected from formyl, acetyl, trifluoroacetyl, benzyl, benzoyl, carbamate, benzyloxycarbonyl, p-methoxybenzyl carbonyl, tert-butyloxycarbonyl (Boc), trimethylsilyl, 2-trimethylsilyl-ethanesulfonyl, trityl and substituted trityl groups, allyloxycarbonyl, 9-fluorenylmethyloxycarbonyl, nitroveratryloxycarbonyl, p-methoxybenzyl and tosyl. In certain embodiments, PG³ is tert-butyloxycarbonyl (Boc).

In some embodiments, PG³ is removed using a deprotecting reagent selected from trifluoroacetic acid, tetra-N-butylammonium fluoride, hydrochloric acid, hydrobromic acid, phosphoric acid, sulfuric acid, methanesulfonic acid, p-toluene sulfonic acid, acetyl chloride, aluminum trichloride, and boron trifluoride. In certain embodiments, PG³ is tert-butyloxycarbonyl (Boc) and the deprotecting agent is hydrochloric acid.

In some embodiments, a process to prepare a compound of formula 17 from a compound of formula 15 includes providing a compound of formula 15 in a crude form or a purified form. In certain embodiments, a compound of formula 15 is provided in a purified form after a crystallization step. In some embodiments, a compound of formula 15 is provided in greater than 95%, 96%, 97%, or greater than 98% purity and greater than 95%, 96%, 97%, 98%, or greater than 99% enantiomeric excess (ee). In some embodiments, a compound of formula 15 is isolated as a solid appropriate for use as an intermediate compound. For instance, in one embodiment, a compound of formula 15 is isolated as a solid appropriate for use in producing a compound of formula 21 or formula 22. In one such embodiment, a compound of formula 15 is a crystalline solid for use in producing a compound of formula 21 or formula 22.

In certain embodiments, the process comprises preparing a compound of formula 17, wherein the compound of formula 15 is:

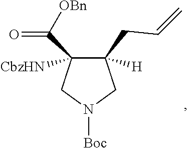 [SMILES: C=CC[C@H]1CN(C(=O)OC(C)(C)C)C[C@@]1(NC(=O)OCc1ccccc1)C(=O)OCc1ccccc1]

3-benzyl 1-(tert-butyl) (3R,4S)-4-allyl-3-(((benzyloxy)carbonyl)amino)pyrrolidine-1,3-dicarboxylate or salt thereof.

In other embodiments, the process for preparing a compound of formula 21 or formula 22 comprises a compound of formula 15*

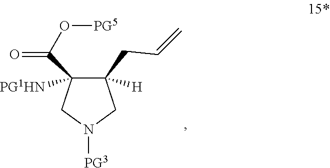

or a salt thereof, wherein:
PG¹ and PG³ are independently suitable amine protecting groups; and
PG⁵ is a suitable carboxylic acid protecting group.

In some embodiments, the process for preparing a compound of formula 21 or formula 22 further comprises preparing the compound of formula 15, the process comprising adding a protecting group to the primary amine of a compound of formula 14

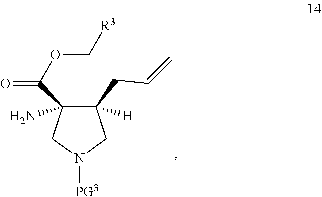

or a salt thereof, wherein:
R³ is a substituted or unsubstituted ring selected from aryl and heteroaryl; and
PG³ is a suitable amine protecting group,
   under suitable conditions to add the protecting group to the primary amine of the compound of formula 14 to form the compound of formula 15 or a salt thereof.

In some embodiments, the suitable conditions used to prepare a compound of formula 15 from a compound of formula 14 include conditions known in the art for adding a protecting group (e.g., PG³) to a primary amine. Suitable amine protecting groups include those defined herein and described above. In certain embodiments, PG³ is benzyloxycarbonyl (Cbz) added using benzyl chloroformate and a base (e.g., a carbonate or amine base).

In some embodiments, a process to prepare a compound of formula 15 from a compound of formula 14 includes providing a compound of formula 14 in a crude form or a purified form. In certain embodiments, a compound of formula 14 is provided in a pure form after one or more extraction, washing, solvent swapping, and crystallization steps. In certain embodiments, the crystallization steps include a resolution step comprising co-crystallization with L(+)-tartaric acid.

In certain embodiments, the process comprises preparing a compound of formula 15, wherein the compound of formula 14 is:

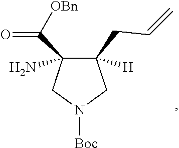 [SMILES: C=CC[C@H]1CN(C(=O)OC(C)(C)C)C[C@@]1(N)C(=O)OCc1ccccc1]

3-benzyl 1-(tert-butyl) (3R,4S)-4-allyl-3-aminopyrrolidine-1,3-dicarboxylate or salt thereof.

In other embodiments, the process for preparing a compound of formula 21 or formula 22 comprises a compound of formula 14*

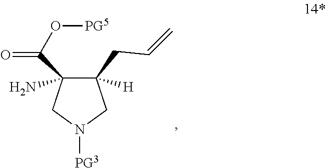

or a salt thereof, wherein:
PG³ is a suitable amine protecting group; and
PG⁵ is a suitable carboxylic acid protecting group.

In some embodiments, the process for preparing a compound of formula 21 or formula 22 further comprises preparing the compound of formula 14, the process comprising reducing the azide of a compound of formula 13

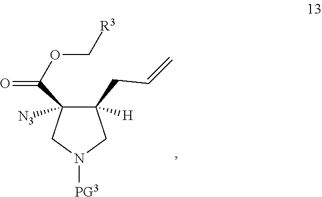

or a salt thereof, wherein:
R³ is a substituted or unsubstituted ring selected from aryl and heteroaryl; and
PG³ is a suitable amine protecting group,
   under suitable conditions to reduce the azide of a compound of formula 13 to form the compound of formula 14 or a salt thereof.

In some embodiments, the suitable conditions used to prepare a compound of formula 14 from a compound of formula 13 include reducing conditions known in the art for converting an azide into a primary amine. In some embodiments, the reducing conditions include the use of hydride donors (e.g., LiAlH₄, NaBH₄, etc.), hydrogen gas or its source in the presence of transition metals and their oxides (MOs) as catalysts, low-valent metal ions (e.g., SnCl₂, CrCl₂, TiCl₃/boranes/silanes, etc.), phosphine reagents (e.g., Staudinger reaction, etc.), and sulfur reagents (e.g., thiolates, etc.). In certain embodiments, the reducing conditions include the use of zinc and acetic acid.

In some embodiments, a process to prepare a compound of formula 14 from a compound of formula 13 includes providing a compound of formula 13 in a crude form or a purified form. In certain embodiments, a compound of formula 13 is provided in a crude form after one or more extraction, washing, and concentration steps. In some embodiments, a compound of formula 13 is provided in greater than 80%, 85%, or greater than 90% purity.

In certain embodiments, the process comprises preparing a compound of formula 14, wherein the compound of formula 13 is:

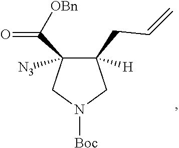 [SMILES: C=CC[C@H]1CN(C(=O)OC(C)(C)C)C[C@@]1(N=[N+]=[N-])C(=O)OCc1ccccc1]

3-benzyl 1-(tert-butyl) (3R,4S)-4-allyl-3-azidopyrrolidine-1,3-dicarboxylate or salt thereof.

In other embodiments, the process for preparing a compound of formula 21 or formula 22 comprises a compound of formula 13*

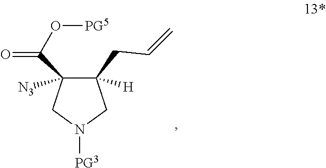

or a salt thereof, wherein:
PG³ is a suitable amine protecting group; and
PG⁵ is a suitable carboxylic acid protecting group.

In some embodiments, the process for preparing a compound of formula 21 or formula 22 further comprises preparing the compound of formula 13 or formula 13*, the process comprising adding a protecting group to the carboxylic acid of a compound of formula 12

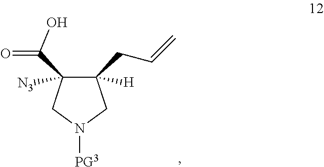

or a salt thereof, wherein:
PG³ is a suitable amine protecting group,
   under suitable conditions to add a protecting group to the carboxylic acid of a compound of formula 12 to form the compound of formula 13 or formula 13* or a salt thereof.

In some embodiments, the suitable conditions used to prepare a compound of formula 13 from a compound of formula 12 include conditions known in the art for adding a protecting group to a carboxylic acid. Suitable carboxylic acid protecting groups and the reagents and reaction conditions appropriate for using them to protect and deprotect carboxylic acids are well known in the art and include those described in detail in Protecting Groups in Organic Synthesis, T. W. Greene and P. G. M. Wuts, 3^{d} edition, John Wiley & Sons, 1999, the entirety of each of which is herein incorporated by reference. In some embodiments, the carboxylic acid protecting group is —CH₂R³, wherein R³ is a substituted or unsubstituted ring selected from aryl and heteroaryl. In certain embodiments, the carboxylic acid protecting group is —CH₂R³, wherein R³ is phenyl (e.g., —CH₂R³ is benzyl). In certain embodiments, —CH₂R³ is benzyl and it is added to the carboxylic acid using a benzyl halide and a base (e.g., a carbonate or amine base).

In some embodiments, a process to prepare a compound of formula 13 from a compound of formula 12 includes providing a compound of formula 12 in a crude form or a purified form. In certain embodiments, a compound of formula 12 is provided in a crude form after one or more extraction, washing, and concentration steps.

In certain embodiments, the process comprises preparing a compound of formula 13, wherein the compound of formula 12 is:

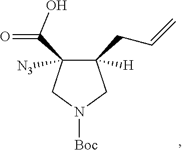 [SMILES: C=CC[C@H]1CN(C(=O)OC(C)(C)C)C[C@@]1(N=[N+]=[N-])C(=O)O]

(3R,4S)-4-allyl-3-azido-1-(tert-butoxycarbonyl)pyrrolidine-3-carboxylic acid or salt thereof.

In some embodiments, the process for preparing a compound of formula 21 or formula 22 further comprises preparing the compound of formula 12 or formula 13, the process comprising treating a compound of formula 11

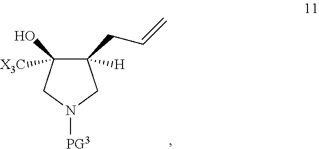

or a salt thereof, wherein:
each X is independently selected from hydrogen or halogen;
PG³ is a suitable amine protecting group,
   under suitable conditions to convert the compound of formula 11 into the compound of formula 12 or a salt thereof.

As defined above and described herein, each X is independently hydrogen or halogen. In some embodiments, X is hydrogen. In some embodiments, X is halogen. In some embodiments, one, two, or three X is fluoro. In some embodiments, one, two, or three X is chloro. In some embodiments, one, two, or three X is bromide. In some embodiments, one, two, or three X is iodo. In some embodiments, each X is chloride. In some embodiments, each X is bromide.

In some embodiments, the suitable conditions used to prepare a compound of formula 12 from a compound of formula 11 includes conditions known in the art for converting a halocarbon to a carboxylic acid and substituting an alcohol with an azide. In some embodiments, the conditions comprise an epoxide intermediate. In some embodiments, the epoxide intermediate provides an electrophilic carbon for azide substitution. In certain embodiments, the conditions used to prepare a compound of formula 12 include sodium azide and a base (e.g., hydroxide). In some embodiments, the conditions further comprise quenching the resulting acid chloride with an alcohol, for example a benzyl alcohol to for a compound of formula 13.

In some embodiments, a process to prepare a compound of formula 12 or formula 13 from a compound of formula 11 includes providing a compound of formula 11 in a crude form or a purified form. In certain embodiments, a compound of formula 11 is provided in a purified form after one or more extraction, washing, solvent swapping, and crystallization steps. In some embodiments, a compound of formula 11 is provided in greater than 95%, 96%, 97%, or greater than 98% purity and greater than 95%, 96%, 97%, 98%, or greater than 99% ee.

In certain embodiments, the process comprises preparing a compound of formula 12, wherein the compound of formula 11 is:

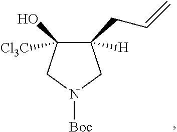

tert-butyl (3S,4S)-4-allyl-3-hydroxy-3-(trichloromethyl)pyrrolidine-1-carboxylate or salt thereof.

In certain embodiments, the process comprises preparing a compound of formula 12, wherein the compound of formula 11 is:

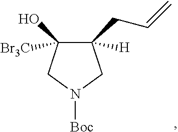

tert-butyl (3S,4S)-4-allyl-3-hydroxy-3-(tribromomethyl)pyrrolidine-1-carboxylate or salt thereof.

In some embodiments, the process for preparing a compound of formula 21 or formula 22 further comprises preparing the compound of formula 11, the process comprising removing the protecting group from the tertiary alcohol of a compound of formula 10

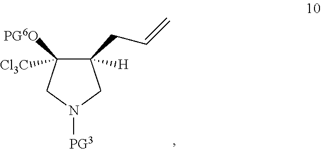

or a salt thereof, wherein:
PG³ is a suitable amine protecting group; and
PG⁶ is a suitable hydroxyl protecting group,
   under suitable conditions to remove the protecting group from the tertiary alcohol of the compound of formula 10 to form the compound of formula 11 or a salt thereof.

In some embodiments, the suitable conditions used to prepare a compound of formula 11 from a compound of formula 10 include conditions known in the art a protecting group (e.g., PG⁶) from a tertiary alcohol. In some embodiments, the conditions used include known conditions for removal (e.g., desilyation) of silicon-based protecting groups. In some embodiments, desilyation occurs under acidic conditions (e.g., using mineral acids, organic acids, etc.) or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like. In certain embodiments, the TMS group of a compound of formula 10 is removed using tetra-N-butylammonium fluoride and acetic acid.

In some embodiments, a process to prepare a compound of formula 11 from a compound of formula 10 includes providing a compound of formula 10 in a crude form or a purified form. In certain embodiments, a compound of formula 10 is provided in a crude form after one or more extraction, washing, and concentration steps. In some embodiments, a compound of formula 10 is provided in greater than 50%, 55%, or greater than 60% purity.

In certain embodiments, the process comprises preparing a compound of formula 11, wherein the compound of formula 10 is:

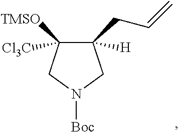

tert-butyl (3S,4S)-4-allyl-3-(trichloromethyl)-3-((trimethylsilyl)oxy)pyrrolidine-1-carboxylate or salt thereof.

In some embodiments, the process for preparing a compound of formula 21 or formula 22 further comprises preparing the compound of formula 10 or formula 11, the process comprising alkylating a compound of formula 9

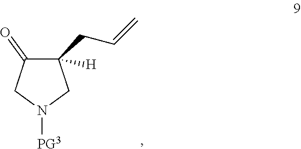

or a salt thereof, wherein:
PG³ is a suitable amine protecting group,
   under suitable conditions to alkylate the compound of formula 9 to form the compound of formula 10 or a salt thereof.

In some embodiments, the suitable conditions used to prepare a compound of formula 10 or formula 11 from a compound of formula 9 include conditions known in the art to alkylate a carbonyl. In some embodiments, the reagent used for alkylation is an alkyl metal nucleophile, such as an alkyl lithium reagent, an alkyl magnesium reagent, an alkyl zinc reagent, an alkyl copper reagent, or reagents including mixtures of these metals. The alkylation may also be assisted with Lewis acids or transitional metals. Solvents can include any of those suitable for nucleophilic addition, such as tetrahydrofuran, 2-methyltetrahydrofuran, N,N-dimethylformamide, and the like. In some embodiments, the reagent used for alkylation comprises functionality that facilitates later carboxylic acid formation. In some embodiments, the alkylation reagent is a trihalomethyltrialkylsilane, such as a trihalomethyltrimethylsilane. In certain embodiments, the alkylation reagent is trichloromethyltrimethylsilane. In some embodiments, the alkylation reagent trichloromethyllithium.

In some embodiments, a process to prepare a compound of formula 10 or formula 11 from a compound of formula 9 includes providing a compound of formula 9 in a crude form or a purified form. In certain embodiments, a compound of formula 9 is provided in a crude form after one or more extraction, washing, and solvent swapping steps. In some embodiments, a compound of formula 9 is provided in greater than 70%, 75%, or greater than 80% purity and greater than 90%, 95%, 96%, or greater than 97% ee.

In certain embodiments, the process comprises preparing a compound of formula 10, wherein the compound of formula 9 is:

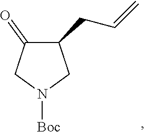 [SMILES: C=CC[C@H]1CN(C(=O)OC(C)(C)C)CC1=O]

tert-butyl (S)-3-allyl-4-oxopyrrolidine-1-carboxylate or salt thereof.

In some embodiments, the process for preparing a compound of formula 21 or formula 22 further comprises preparing the compound of formula 9, the process comprising oxidizing the secondary alcohol of a compound of formula 8

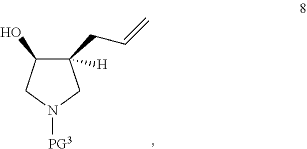

or a salt thereof, wherein:
PG³ is a suitable amine protecting group,
   under suitable conditions to oxidize the secondary alcohol of a compound of formula 8 to form the compound of formula 9 or a salt thereof.

In some embodiments, the suitable conditions used to prepare a compound of formula 9 from a compound of formula 8 include conditions known in the art to oxidize a secondary alcohol to a ketone. In some embodiments, the conditions include known methods readily apparent to those skill in the art for oxidizing secondary alcohols, such as but not limited to, Swern oxidation, Parikh-Doering oxidation, Corey-Kim oxidation, oxidation using hypervalent iodine (e.g., IBX and DMP), and the like. In certain embodiments, the conditions used are Swern oxidation conditions (e.g., using oxalyl chloride and DMSO). In some embodiments, the conditions include a hindered base to prevent racemization of the compound of formula 9. In some embodiments, use of a hindered base (e.g., Hunig's base) provides the compound of formula 9 in greater than 75%, 80%, 85%, 90%, 95%, 96%, 97%, or even 99% ee.

In some embodiments, a process to prepare a compound of formula 9 from a compound of formula 8 includes providing a compound of formula 8 in a crude form or a purified form. In certain embodiments, a compound of formula 8 is provided in a pure form after one or more extraction, washing, solvent swapping, and crystallization steps. In some embodiments, a compound of formula 8 is provided in greater than 90%, 95%, 96%, 97%, 98%, or greater than 99% purity.

In certain embodiments, the process comprises preparing a compound of formula 9, wherein the compound of formula 8 is:

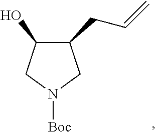 [SMILES: C=CC[C@H]1CN(C(=O)OC(C)(C)C)C[C@H]1O]

tert-butyl (3S,4S)-3-allyl-4-hydroxypyrrolidine-1-carboxylate or salt thereof.

In some embodiments, the process for preparing a compound of formula 21 or formula 22 further comprises preparing the compound of formula 8, the process comprising removing the protecting group from the secondary alcohol and the initial protecting group (i.e., PG^{N}, if PG^{N} is a suitable amine protecting group) from the secondary amine, and adding another protecting group to the secondary amine of a compound of formula 7

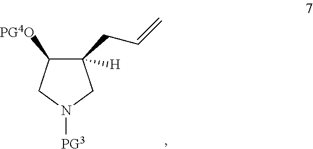

or a salt thereof, wherein:
PG⁴ is a suitable hydroxyl protecting group; and
PG^{N} is H or a suitable amine protecting group,
   under suitable conditions to remove the protecting group from the secondary alcohol and the initial protecting group (i.e., PG^{N}, if PG^{N} is a suitable amine protecting group) from the secondary amine, and adding another protecting group to the secondary amine of a compound of formula 7 to form the compound of formula 8 or a salt thereof.

In some embodiments, the suitable conditions used to prepare a compound of formula 8 from a compound of formula 7 include conditions known in the art to remove a protecting group (e.g., PG⁴) from a secondary alcohol and at the same time remove a protecting group (e.g., PG^{N}, if PG^{N} is a suitable amine protecting group, such as —CH₂—(C₆-C₁₀)aryl or —CH₂—(C₅-C₉)heteroaryl, such as Bn) from a secondary amine, if present. In some embodiments, the conditions include reductive conditions, oxidative conditions, or using a Lewis acid. In some embodiments, the conditions include lithium in liquid ammonia, hydrogenolysis with compositions comprising palladium, nickel, or platinum, etc., chromium oxide and acidic acid, ozone, N-bromosuccinimide (NBS), N-iodosuccinimide (NIS), trimethylsilyl iodide, and haloalkyl chloroformates. In certain embodiments, the conditions include 1-chloroethyl chloroformate.

In some embodiments, after deprotection of the secondary amine, the suitable conditions used to prepare a compound of formula 8 include conditions known in the art for adding a protecting group (e.g., PG³) to a secondary amine. Suitable amine protecting groups include those defined herein and described above. In certain embodiments, PG³ is tert-butyloxycarbonyl (Boc). In some embodiments, the tert-butyloxycarbonyl (Boc) group is added using di-tert-butyl dicarbonate (Boc₂O) and a base (e.g., a carbonate or amine base).

In some embodiments, a process to prepare a compound of formula 8 from a compound of formula 7 includes providing a compound of formula 7 in a crude form or a purified form. In certain embodiments, a compound of formula 7 is provided in a crude form after one or more extraction, washing, and concentration steps. In some embodiments, a compound of formula 7 is provided in greater than 75%, 80%, 85%, or greater than 90% purity.

In certain embodiments, the process comprises preparing a compound of formula 8, wherein the compound of formula 7 is:

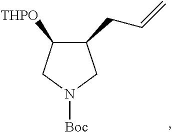

(3S,4S)-3-allyl-1-benzyl-4-((tetrahydro-2H-pyran-2-yl)oxy)pyrrolidine or salt thereof.

In some embodiments, the process for preparing a compound of formula 21 or formula 22 further comprises preparing the compound of formula 7, the process comprising treating a compound of formula 6

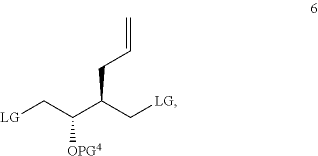

or a salt thereof, with H₂N-PG^{N}, wherein:
LG is a suitable leaving group;
PG⁴ is a suitable hydroxyl protecting group; and
PG^{N} is H or a suitable amine protecting group,
   under suitable conditions to perform a double displacement reaction on the compound of formula 6 to form the compound of formula 7 or a salt thereof.

As defined above and described herein, LG is a suitable leaving group. In some embodiments, LG is halogen. In some embodiments, LG is mesylate. In some embodiments, LG is tosylate. In other embodiments, LG is triflate.

In some embodiments, PG^{N} is hydrogen, and H₂N-PG^{N} is therefore ammonia. In some embodiments, PG^{N} is a suitable amine protecting group. In some embodiments, PG^{N} is a suitable amine protecting group selected from an alkylene-aryl or alkylene-heteroaryl group, such as —CH₂—(C₆-C₁₀)aryl or —CH₂—(C₅-C₉)heteroaryl. In certain embodiments, PG^{N} is benzyl, and H₂N-PG^{N} is therefore benzylamine.

In certain embodiments, the suitable conditions used to prepare a compound of formula 7 from a compound of formula 6 include conditions known in the art to facilitate a substitution reaction with amines. Solvents can include any of those suitable for substitution reactions, such as dichloromethane, tetrahydrofuran, 2-methyltetrahydrofuran, N,N-dimethylformamide, or the reaction may be run in neat amine or with other unreactive amines, such as a tertiary amine. In certain embodiments, the solvent is a tertiary amine, such as diisopropylethylamine.

In some embodiments, a process to prepare a compound of formula 7 from a compound of formula 6 includes providing a compound of formula 6 in a crude form or a purified form. In certain embodiments, a compound of formula 6 is provided in a crude form after one or more extraction, washing, and concentration steps.

In certain embodiments, the process comprises preparing a compound of formula 7, wherein the compound of formula 6 is:

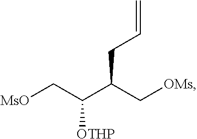

(2S,3S)-2-allyl-3-((tetrahydro-2H-pyran-2-yl)oxy)butane-1,4-diyl dimethanesulfonate or salt thereof.

In some embodiments, the process for preparing a compound of formula 21 or formula 22 further comprises preparing the compound of formula 6, the process comprising converting the primary alcohols of a compound of formula 5

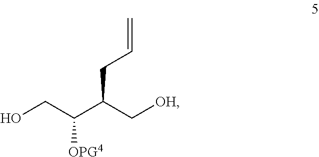

or a salt thereof, wherein:
PG⁴ is a suitable hydroxyl protecting group,
   into leaving groups under suitable conditions to form the compound of formula 6 or a salt thereof.

In some embodiments, the suitable conditions used to prepare a compound of formula 6 from a compound of formula 5 include conditions known in the art to convert a primary alcohol into a leaving group. In some embodiments, the conditions include reacting a primary alcohol to form a halogen (e.g., deoxychlorination or deoxybromination). In certain embodiments, the conditions include reacting a primary alcohol to form a sulfonate (e.g., mesylation, triflation, or tosylation using methanesulfonyl chloride, tosyl chloride, or triflic anhydride, respectively). Solvents can include any of those suitable for substitution reactions, such as dichloromethane, tetrahydrofuran, 2-methyltetrahydrofuran, N,N-dimethylformamide, or the like. In certain embodiments, the conditions include mesylation in dichloromethane and using a base (e.g., a carbonate or amine base).

In some embodiments, a process to prepare a compound of formula 6 from a compound of formula 5 includes providing a compound of formula 5 in a crude form or a purified form. In certain embodiments, a compound of formula 5 is provided in a crude form after one or more extraction, washing, and concentration steps. In some embodiments, a compound of formula 5 is provided in greater than 70%, 75%, 80%, or greater than 85% purity.

In certain embodiments, the process comprises preparing a compound of formula 6, wherein the compound of formula 5 is:

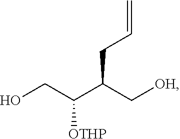

(2S,3S)-2-allyl-3-((tetrahydro-2H-pyran-2-yl)oxy)butane-1,4-diol or salt thereof.

In some embodiments, the process for preparing a compound of formula 21 or formula 22 further comprises preparing the compound of formula 5, the process comprising reducing the ethyl carboxylates of a compound of formula 4

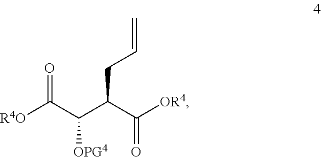

or a salt thereof, wherein:
each R⁴ is independently (C₁-C₆)alkyl;
PG⁴ is a suitable hydroxyl protecting group,
   under suitable conditions to reduce the alkyl carboxylates of the compound of formula 4 to form the compound of formula 5 or a salt thereof.

As defined above and described herein, each R⁴ is independently (C₁-C₆)alkyl. In some embodiments, R⁴ is methyl. In some embodiments, R⁴ is ethyl. In some embodiments, R⁴ is isopropyl.

In some embodiments, the suitable conditions used to prepare a compound of formula 5 from a compound of formula 4 include conditions known in the art to reduce carboxylates. In some embodiments, the conditions include a reducing agent selected from a borane, an alkali metal borohydride, or an alkali metal aluminum hydride. In some embodiments, the reducing agent is selected from borane-dimethyl sulfide, sodium borohydride, lithium borohydride, potassium borohydride, lithium aluminum hydride, diisobutylaluminum hydride, or sodium bis(2-methoxyethoxy)aluminum hydride (Red Al®). Solvents can include any of those suitable for reductions, such as dichloromethane, tetrahydrofuran, 2-methyltetrahydrofuran, toluene, or the like. In certain embodiments, the reducing agent is sodium bis(2-methoxyethoxy)aluminum hydride (Red Al®) and solvent is toluene.

In some embodiments, a process to prepare a compound of formula 5 from a compound of formula 4 includes providing a compound of formula 4 in a crude form or a purified form. In certain embodiments, a compound of formula 4 is provided in a crude form after one or more extraction, washing, and concentration steps. In some embodiments, a compound of formula 4 is provided in greater than 60%, 65%, 70%, or greater than 75% purity.

In certain embodiments, the process comprises preparing a compound of formula 5, wherein the compound of formula 4 is:

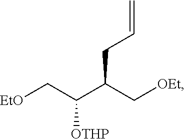

2-(((2S,3S)-1-ethoxy-3-(ethoxymethyl)hex-5-en-2-yl)oxy)tetrahydro-2H-pyran or salt thereof.

In some embodiments, the process for preparing a compound of formula 21 or formula 22 further comprises preparing the compound of formula 4, the process comprising adding a protecting group to the secondary alcohol of a compound of formula 3

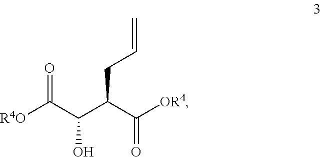

or a salt thereof, wherein:
each R⁴ is independently (C₁-C₆)alkyl;
   under suitable conditions to add the protecting group to the secondary alcohol of the compound of formula 3 to form the compound of formula 4 or a salt thereof.

In some embodiments, the suitable conditions used to prepare a compound of formula 4 from a compound of formula 3 include conditions known in the art for protecting a secondary alcohol. In some embodiments, the hydroxyl protecting group is 2-tetrahydropyranyl and the THP ether can be generated using 3,4-dihydropyran (DHP) under acidic conditions (e.g., using mineral acids, organic acids, etc.) or using the Mitsunobu reaction, such as using hydroxytetrahydropyranyl, triphenylphosphine, and diethyl azodicarboxylate (DEAD) in tetrahydrofuran. In certain embodiments, the conditions include reacting the secondary alcohol with 3,4-dihydropyran (DHP) in dichloromethane and catalytic pyridinium p-toluenesulfonate (PPTS).

In some embodiments, a process to prepare a compound of formula 4 from a compound of formula 3 includes providing a compound of formula 3 in a crude form or a purified form. In certain embodiments, a compound of formula 3 is provided in a crude form after one or more extraction, washing, and concentration steps. In some embodiments, a compound of formula 3 is provided in greater than 60%, 65%, 70%, or greater than 75% purity and greater than 75:25, 80:20, 85:15, or greater than 90:10 dr.

In some embodiments, the process for preparing a compound of formula 21 or formula 22 further comprises preparing the compound of formula 3, the process comprising allylating a compound of formula 2

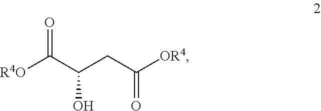

or a salt thereof, wherein:
each R⁴ is independently (C₁-C₆)alkyl;
   under suitable conditions to allylate the compound of formula 2 to form the compound of formula 3 or a salt thereof.

In some embodiments, the suitable conditions used to prepare a compound of formula 3 from a compound of formula 2 include conditions known in the art for allylating an alpha/beta unsaturated ester. In some embodiments, the conditions include forming the enolate and treating with an allyl electrophile. In certain embodiments, the enolate is formed with lithium HMDS and the allyl electrophile is an allyl halide, such as allylbromide.

In some embodiments, a process to prepare a compound of formula 3 from a compound of formula 2 includes providing a compound of formula 2 in a crude form or a purified form. In certain embodiments, a compound of formula 2 is provided in a crude form after one or more extraction, washing, and concentration steps. In some embodiments, a compound of formula 2 is provided in greater than 75%, 80%, 85%, or greater than 90% purity.

In some embodiments, the process for preparing a compound of formula 21 or formula 22 further comprises preparing the compound of formula 2, the process comprising esterifying L-malic acid under suitable conditions to form the compound of formula 2 or a salt thereof.

In some embodiments, the suitable conditions used to prepare a compound of formula 2 from L-malic acid include known conditions in the art to esterify a carboxylic acid. In some embodiments, the conditions include esterifying directly with an alcohol solvent under acidic conditions (e.g., using mineral acids, organic acids, etc.). In certain embodiments, the conditions include treating with acetyl chloride and a (C₁-C₆)alkyl alcohol, such as ethanol.

### 3.3. Alternate Process to Prepare a Compound of Formula 8

Provided herein is an alternate process for preparing a compound of formula 8:

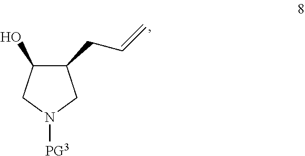

or a salt thereof, wherein:
PG³ is a suitable amine protecting group.

In one embodiment, the present methods provide a process of preparing a compound of formula 8, wherein the compound is:

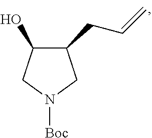 [SMILES: C=CC[C@H]1CN(C(=O)OC(C)(C)C)C[C@H]1O]

tert-butyl (3S,4S)-3-allyl-4-hydroxypyrrolidine-1-carboxylate or a pharmaceutically acceptable salt thereof.

In some embodiments, a compound of formula 8 is prepared according to Scheme 2.

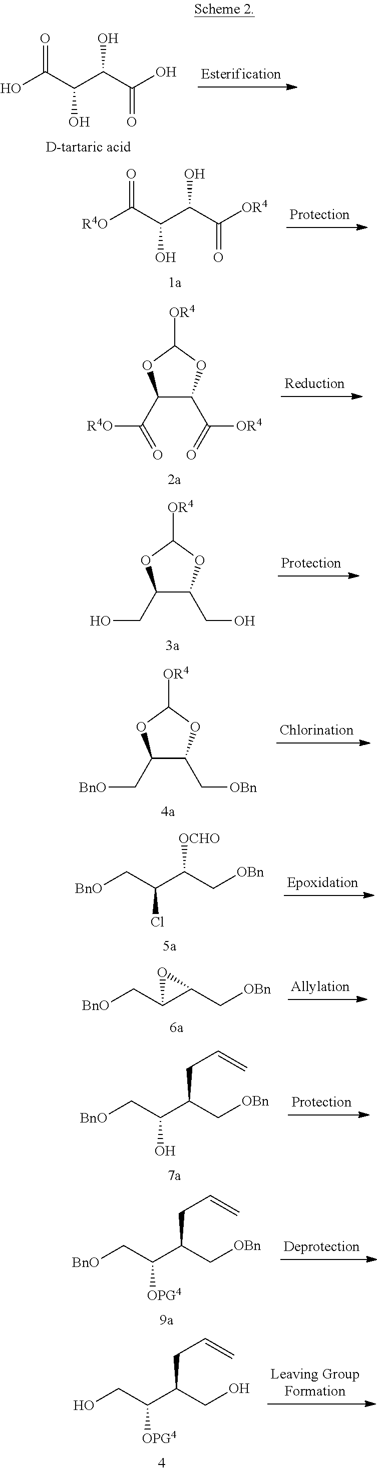

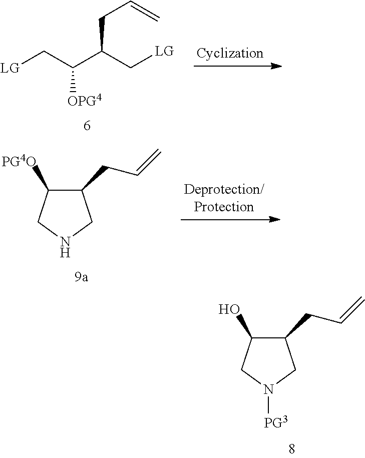

In Scheme 2 above, the reactions steps and variables LG, R⁴, PG³, and PG⁴ are defined below and described herein.

In some embodiments, the process for preparing a compound of formula 21 or formula 22 comprises preparing the compound of formula 8, the process comprising removing the protecting group from the secondary alcohol of a compound of formula 9a

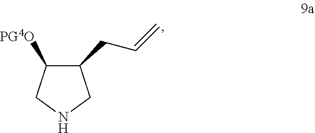

or a salt thereof, wherein:
PG⁴ is a suitable hydroxyl protecting group, and
   adding a protecting group to the secondary amine under suitable conditions to form the compound of formula 9 or a salt thereof.

In some embodiments, the suitable conditions used to prepare a compound of formula 9 from a compound of formula 8 include conditions known in the art to deprotect a secondary alcohol and protect a secondary amine. Suitable hydroxyl protecting groups include those defined herein and described above. In some embodiments, the conditions to deprotect the secondary alcohol include acidic conditions, basic conditions, a Lewis acid, or mixtures thereof In certain embodiments, PG⁴ is THP and it is removed under acidic conditions (e.g., mineral or organic acid). In some embodiments, after deprotection of the secondary alcohol, the suitable conditions used to prepare a compound of formula 8 include conditions known in the art for adding a protecting group (e.g., PG³) to a secondary amine. Suitable amine protecting groups include those defined herein and described above. In certain embodiments, PG³ is tert-butyloxycarbonyl (Boc). In some embodiments, the tert-butyloxycarbonyl (Boc) group is added using di-tert-butyl dicarbonate (Boc₂O) and an optional base (e.g., a carbonate or amine base).

In some embodiments, a process to prepare a compound of formula 8 from a compound of formula 9a includes providing a compound of formula 9a in a crude form or a purified form. In certain embodiments, a compound of formula 9a is provided in a crude form after one or more extraction, washing, and concentration steps.

In some embodiments, the process for preparing a compound of formula 21 or formula 22 further comprises preparing the compound of formula 9a, the process comprising treating a compound of formula 6

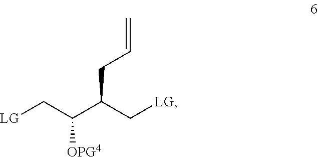

or a salt thereof, wherein:
LG is a suitable leaving group;
PG⁴ is a suitable hydroxyl protecting group,
   with an ammonia source under suitable conditions to form the compound of formula 9a or a salt thereof.

In some embodiments, the suitable conditions used to prepare a compound of formula 9a from a compound of formula 6 include conditions known in the art to facilitate a substitution reaction with an ammonia source. In some embodiments, the ammonia source is ammonia or an ammonium salt. In certain embodiments, the conditions include pressurized ammonia.

In some embodiments, a process to prepare a compound of formula 9a from a compound of formula 6 includes providing a compound of formula 6 in a crude form or a purified form. In certain embodiments, a compound of formula 6 is provided in a crude form after one or more extraction, washing, and concentration steps.

In some embodiments, the process for preparing a compound of formula 21 or formula 22 further comprises preparing the compound of formula 6, the process comprising mesylating the primary alcohols of a compound of formula 4

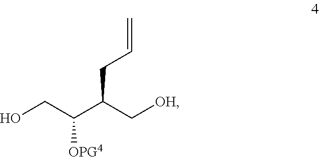

or a salt thereof, wherein:
PG⁴ is a suitable hydroxyl protecting group,
   under suitable conditions to form the compound of formula 6 or a salt thereof.

In some embodiments, the suitable conditions used to prepare a compound of formula 6 from a compound of formula 4 include conditions known in the art to facilitate mesylation of an alcohol. Solvents can include any of those suitable for substitution reactions, such as dichloromethane, tetrahydrofuran, 2-methyltetrahydrofuran, N,N-dimethylformamide, or the like. In certain embodiments, the conditions include dichloromethane and a base (e.g., a carbonate or amine base).

In some embodiments, a process to prepare a compound of formula 6 from a compound of formula 4 includes providing a compound of formula 4 in a crude form or a purified form. In certain embodiments, a compound of formula 4 is provided in a crude form after one or more extraction, washing, and concentration steps.

In some embodiments, the process for preparing a compound of formula 21 or formula 22 further comprises preparing the compound of formula 4, the process comprising removing the benzyl groups from the primary alcohols of a compound of formula 8a

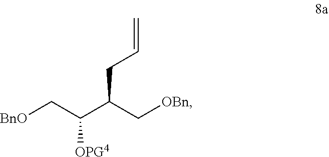

or a salt thereof, wherein:
PG⁴ is a suitable hydroxyl protecting group,
   under suitable conditions to form the compound of formula 4 or a salt thereof.

In some embodiments, the suitable conditions used to prepare a compound of formula 4 from a compound of formula 8a include conditions known in the art to remove a benzyl group from a primary alcohol. In some embodiments, the conditions include reductive conditions, oxidative conditions, or using a Lewis acid. In some embodiments, the conditions include lithium in liquid ammonia, hydrogenolysis with compositions comprising palladium, nickel, or platinum, etc., chromium oxide and acidic acid, ozone, N-bromosuccinimide (NBS), N-iodosuccinimide (NIS), trimethylsilyl iodide, and haloalkyl chloroformates. In certain embodiments, the conditions include lithium in liquid ammonia and THF.

In some embodiments, a process to prepare a compound of formula 4 from a compound of formula 8a includes providing a compound of formula 8a in a crude form or a purified form. In certain embodiments, a compound of formula 8a is provided in a crude form after one or more extraction, washing, and concentration steps.

In some embodiments, the process for preparing a compound of formula 21 or formula 22 further comprises preparing the compound of formula 8a, the process comprising adding a protecting group to the secondary alcohol a compound of formula 7a

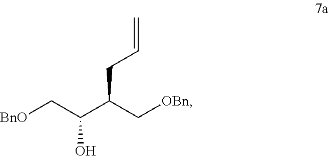 [SMILES: C=CC[C@@H](COCc1ccccc1)[C@H](O)COCc1ccccc1]

or a salt thereof,
under suitable conditions to form the compound of formula 8a or a salt thereof.

In some embodiments, the suitable conditions used to prepare a compound of formula 8a from a compound of formula 7a include conditions known in the art to protect a secondary alcohol. In some embodiments, the hydroxyl protecting group is as defined above and described herein. In certain embodiments, the hydroxyl protecting group is 2-tetrahydropyranyl (THP). In some embodiments, the THP ether can be generated using 3,4-dihydropyran (DHP) under acidic conditions (e.g., using mineral acids, organic acids, etc.) or using the Mitsunobu reaction, such as using hydroxytetrahydropyranyl, triphenylphosphine, and diethyl azodicarboxylate (DEAD) in tetrahydrofuran. In certain embodiments, the conditions include reacting the secondary alcohol with 3,4-dihydropyran (DHP) in the presence of an acid catalyst (e.g., PPTS).

In some embodiments, a process to prepare a compound of formula 8a from a compound of formula 7a includes providing a compound of formula 7a in a crude form or a purified form. In certain embodiments, a compound of formula 7a is provided in a purified form after one or more extraction, washing, and concentration steps. In some embodiments, a compound of formula 7a is provided in greater than 90%, 94%, 95%, 96%, or greater than 97% purity.

In some embodiments, the process for preparing a compound of formula 21 or formula 22 further comprises preparing the compound of formula 7a, the process comprising allylating a compound of formula 6a

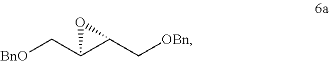 [SMILES: c1ccc(COC[C@H]2O[C@@H]2COCc2ccccc2)cc1]

or a salt thereof,
under suitable conditions to form the compound of formula 7a or a salt thereof.

In some embodiments, the suitable conditions used to prepare a compound of formula 7a from a compound of formula 6a include conditions known in the art to facilitate nucleophilic epoxide ring opening and protection of the resulting secondary alcohol. Allylation of epoxide 6a can be accomplished using an appropriate allyl metal nucleophile, such as an allyl lithium reagent, an ally magnesium reagent, an allyl zinc reagent, an allyl copper reagent, or reagents including mixtures of these metals. The epoxide ring opening may also be assisted with Lewis acids or transitional metals. Solvents can include any of those suitable for nucleophilic addition, such as dioxane, tetrahydrofuran, 2-methyltetrahydrofuran, toluene, and the like. In certain embodiments, the allyl metal nucleophile is allylmagnesium bromide. In some embodiments, the resulting ring-opened epoxide can be protected in situ or by a subsequent reaction.

In some embodiments, a process to prepare a compound of formula 7a from a compound of formula 6a includes providing a compound of formula 6a in a crude form or a purified form. In certain embodiments, a compound of formula 6a is provided in a purified form after one or more extraction, washing, solvent swapping, and crystallization steps. In some embodiments, a compound of formula 6a is provided in greater than 95%, 96%, 97%, 98%, or greater than 99% purity.

In some embodiments, the process for preparing a compound of formula 21 or formula 22 further comprises preparing the compound of formula 6a, the process comprising treating a compound of formula 5a

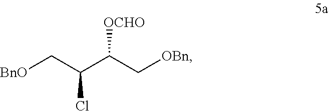

or a salt thereof,
with a base under suitable conditions to form the compound of formula 6a or a salt thereof.

In some embodiments, the suitable conditions used to prepare a compound of formula 6a from a compound of formula 5a include conditions known in the art to facilitate epoxide formation. In some embodiments, the base used to facilitate epoxide formation includes carbonates, hydroxides, amines, and the like. In certain embodiments, the base is potassium carbonate.

In some embodiments, a process to prepare a compound of formula 6a from a compound of formula 5a includes providing a compound of formula 5a in a crude form or a purified form. In certain embodiments, a compound of formula 5a is provided in a crude form after one or more extraction, washing, and concentration steps.

In some embodiments, the process for preparing a compound of formula 21 or formula 22 further comprises preparing the compound of formula 5a, the process comprising chlorinating a compound of formula 4a

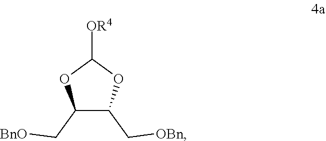

or a salt thereof, wherein:
R⁴ is (C₁-C₆)alkyl,
   with under suitable conditions to form the compound of formula 5a or a salt thereof.

In some embodiments, the suitable conditions used to prepare a compound of formula 5a from a compound of formula 4a include conditions known in the art to facilitate chlorination of C—O bonds. In certain embodiments, the conditions include phosphorus pentachloride.

In some embodiments, a process to prepare a compound of formula 5a from a compound of formula 4a includes providing a compound of formula 4a in a crude form or a purified form. In certain embodiments, a compound of formula 4a is provided in a crude form after one or more extraction, washing, and concentration steps.

In some embodiments, the process for preparing a compound of formula 21 or formula 22 further comprises preparing the compound of formula 4a, the process comprising adding benzyl groups to the primary alcohols of a compound of formula 3a

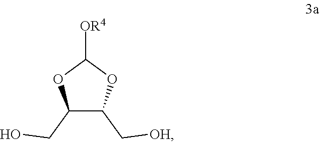

or a salt thereof, wherein:
R⁴ is (C₁-C₆)alkyl,
   under suitable conditions to form the compound of formula 4a or a salt thereof.

In some embodiments, the suitable conditions used to prepare a compound of formula 4a from a compound of formula 3a include conditions known in the art for adding benzyl group to a primary alcohol. In certain embodiments, benzyl groups are added to the primary alcohols using a benzyl halide (e.g., BnCl or BnBr) and a base (e.g., a carbonate, amine, or hydride base).

In some embodiments, a process to prepare a compound of formula 4a from a compound of formula 3a includes providing a compound of formula 3a in a crude form or a purified form. In certain embodiments, a compound of formula 3a is provided in a crude form after one or more filtration and concentration steps. In some embodiments, a compound of formula 3a is provided in greater than 75%, 80%, 85%, or greater than 90% purity.

In some embodiments, the process for preparing a compound of formula 21 or formula 22 further comprises preparing the compound of formula 3a, the process comprising reducing the ethyl carboxylates of a compound of formula 2a

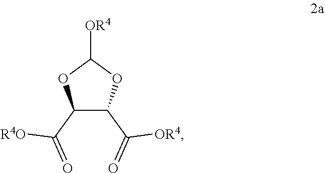

or a salt thereof, wherein:
each R⁴ is independently (C₁-C₆)alkyl,
   under suitable conditions to form the compound of formula 3a or a salt thereof.

In some embodiments, the suitable conditions used to prepare a compound of formula 3a from a compound of formula 2a include conditions known in the art to reduce alkyl carboxylates. In some embodiments, the conditions include a reducing agent selected from a borane, an alkali metal borohydride, or an alkali metal aluminum hydride. In some embodiments, the reducing agent is selected from borane-dimethyl sulfide, sodium borohydride, lithium borohydride, potassium borohydride, lithium aluminum hydride, diisobutylaluminum hydride, or sodium bis(2-methoxyethoxy)aluminum hydride (Red Al©). Solvents can include any of those suitable for reductions, such as alcohols, dichloromethane, tetrahydrofuran (THF), 2-methyltetrahydrofuran, toluene, or the like. In certain embodiments, the reducing agent is lithium aluminum hydride (LAH) and solvent is THF.

In some embodiments, a process to prepare a compound of formula 3a from a compound of formula 2a includes providing a compound of formula 2a in a crude form or a purified form. In certain embodiments, a compound of formula 2a is provided in a crude form after one or more extraction, washing, and concentration steps. In some embodiments, a compound of formula 2a is provided in greater than 80%, 85%, 90%, or greater than 95% purity.

In some embodiments, the process for preparing a compound of formula 21 or formula 22 further comprises preparing the compound of formula 2a, the process comprising treating a compound of formula 1a

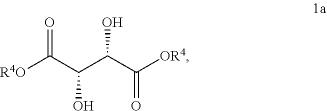

or a salt thereof, wherein:
each R⁴ is independently (C₁-C₆)alkyl,
   with an ethylformate source under suitable conditions to form the compound of formula 2a or a salt thereof.

In some embodiments, the suitable conditions used to prepare a compound of formula 2a from a compound of formula 1a include conditions known in the art to protect diols with an alkylformate source. In some embodiments, the conditions include using triethyl orthoformate under acidic conditions (e.g., mineral acid or organic acid).

In some embodiments, a process to prepare a compound of formula 2a from a compound of formula 1a includes providing a compound of formula 1a in a crude form or a purified form. In certain embodiments, a compound of formula 1a is provided in a crude form after one or more extraction, washing, and concentration steps.

In some embodiments, the process for preparing a compound of formula 21 or formula 22 further comprises preparing the compound of formula 1a, the process comprising esterifying D-tartaric acid under suitable conditions to form the compound of formula 1a or a salt thereof.

In some embodiments, the suitable conditions used to prepare a compound of formula 1a from D-tartaric acid include known conditions in the art to esterify a carboxylic acid. In some embodiments, the conditions include esterifying directly with an alcohol solvent under acidic conditions (e.g., using mineral acids, organic acids, etc.). In certain embodiments, the conditions include treating with acetyl chloride and a (C₁-C₆)alkyl alcohol, such as ethanol.

### 3.4. Alternate Process to Prepare a Compound of Formula 9

Provided herein is an alternate process for preparing a compound of formula 9:

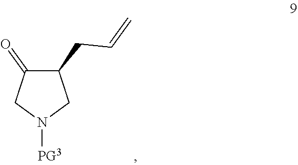

or a salt thereof, wherein:
PG³ is a suitable amine protecting group.

In one embodiment, the present methods provide a process of preparing a compound of formula 9, wherein the compound is:

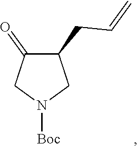 [SMILES: C=CC[C@H]1CN(C(=O)OC(C)(C)C)CC1=O]

tert-butyl (S)-3-allyl-4-oxopyrrolidine-1-carboxylate or a salt thereof.

In some embodiments, a compound of formula 9 is prepared according to Scheme 3.

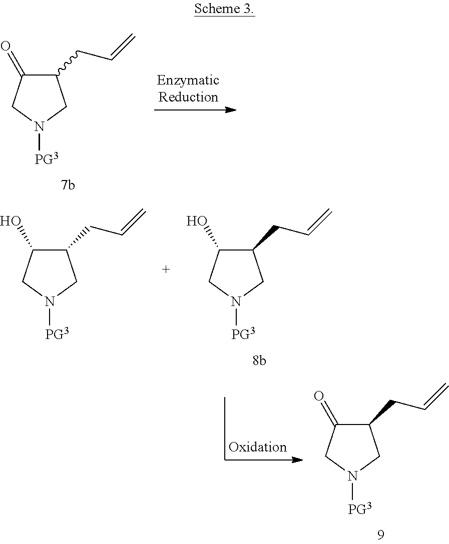

In Scheme 3 above, the reactions steps and variable PG³ are defined below and described herein.

In some embodiments, the process for preparing a compound of formula 21 or formula 22 further comprises preparing the compound of formula 9, the process comprising oxidizing the secondary alcohol of a compound of formula 8b

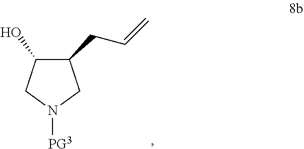

or a salt thereof, wherein:
PG³ is a suitable amine protecting group,
   under suitable conditions to form the compound of formula 9 or a salt thereof.

In some embodiments, the suitable conditions used to prepare a compound of formula 9 from a compound of formula 8b include conditions known in the art to oxidize a secondary alcohol to a ketone. In some embodiments, the conditions include known methods readily apparent to those skill in the art for oxidizing secondary alcohols, such as but not limited to, Swern oxidation, Parikh-Doering oxidation, Corey-Kim oxidation, oxidation using hypervalent iodine (e.g., IBX and DMP), and the like. In certain embodiments, the conditions used are Swern oxidation conditions (e.g., using oxalyl chloride and DMSO).

In some embodiments, the process for preparing a compound of formula 21 or formula 22 further comprises preparing the compound of formula 8b, the alternative process comprising diastereoselective enzymatic reduction of a compound of formula 7b

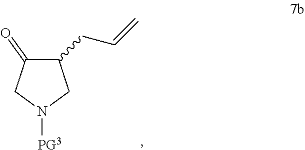

or a salt thereof, wherein:
PG³ is a suitable amine protecting group,
   under suitable conditions to form the compound of formula 8b or a salt thereof.

In some embodiments, the suitable conditions used to prepare a compound of formula 8b from a compound of formula 7b include conditions known in the art for diastereoselective enzymatic reduction of a ketone to a secondary alcohol. In some embodiments, the conditions include known NADPH-dependent ketone reductases (KREDs) that display high diastereoselectivity in reactions of α-substituted ketones. KRED enzymes can be found in a wide range of bacteria and yeasts (See e.g., Kraus and Waldman, Enzyme catalysis in organic synthesis Vols. 1&2. VCH Weinheim 1995; Faber, K., Biotransformations in organic chemistry, 4th Ed. Springer, Berlin Heidelberg New York. 2000; and Hummel and Kula, 1989, Eur. J. Biochem. 184:1-13). In certain embodiments, the conditions include KRED-208 using NADP in aqueous alcohol.

In particular embodiments, the enzymatic reduction of a compound of formula 7b to form a compound of formula 8b is highly diastereoselective. In some embodiments the diastereomeric ratio (dr) of the (R,S) isomer to the (S,S) isomer is greater than 90:10, 91:9, 92:8, 93:7, 94:6, 95:5, 96:4, or even greater than 97:3 dr. In certain embodiments, a compound of formula 8b is formed with 97:3 dr.

In certain embodiments, the alternate process comprises preparing a compound of formula 8b, wherein the compound is:

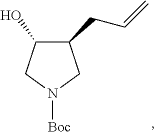 [SMILES: C=CC[C@H]1CN(C(=O)OC(C)(C)C)C[C@@H]1O]

tert-butyl (3S,4R)-3-allyl-4-hydroxypyrrolidine-1-carboxylate or salt thereof.

### 3.5. Alternate Processes to Prepare a Compound of Formula 22

Provided herein are alternate processes for preparing a compound of formula 22:

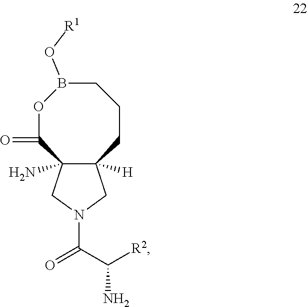

or a pharmaceutically acceptable salt thereof, wherein:
R¹ is H or (C₁-C₆)alkyl;
R² is H, (C₁-C₆)alkyl, —CH₂OZ, —CH(CH₃)OZ, —CH₂SZ, —(CH₂)₂SCH₃—CH₂CONZ₂, —(CH₂)₂CONZ₂, —CH₂CO₂Z, —(CH₂)₂CO₂Z, —(CH₂)₄NZ₂,

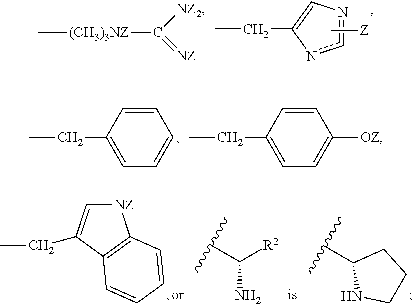

each Z is independently H, (C₁-C₆)alkyl, or a suitable protecting group.

3.5.1. Alternate Route A

In some embodiments, a compound of formula 22 is prepared according to Scheme 4.

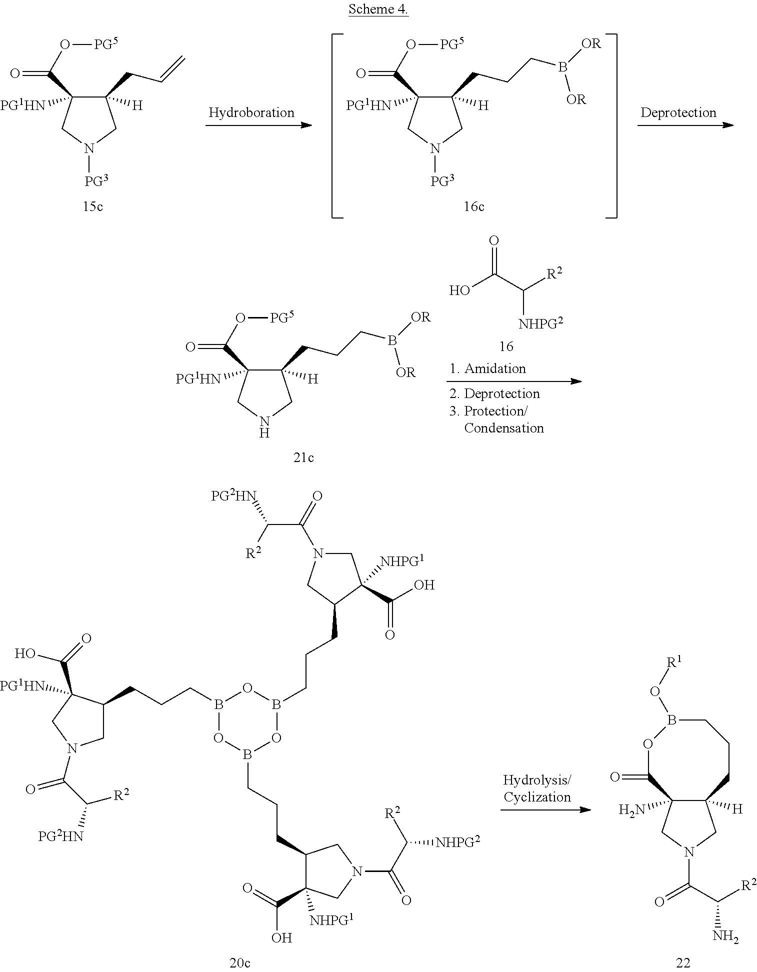

In Scheme 4 above, the reactions steps and variables R, R¹, R², PG¹, PG², PG³, and PG⁵ are defined above and described herein.

In some embodiments, the alternate process of preparing a compound of formula 22 or a pharmaceutically acceptable salt thereof comprises treating a compound of formula 20c

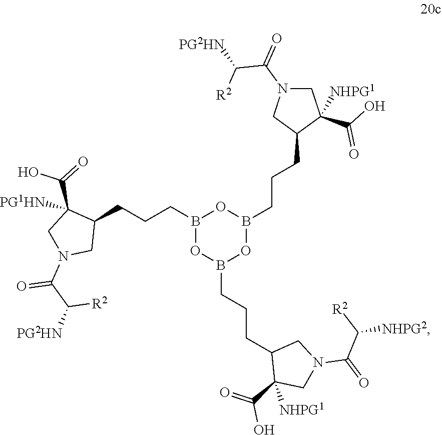

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

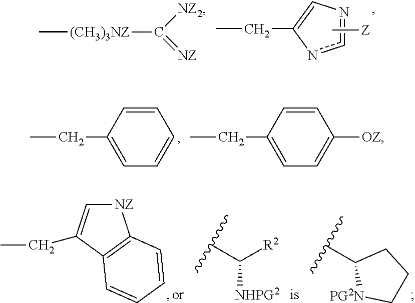

each Z is independently H, (C₁-C₆)alkyl, or a suitable protecting group; and
PG¹ and PG² are independently suitable amine protecting groups,
   under suitable conditions to form the compound of formula 22 or a pharmaceutically acceptable salt thereof.

In some embodiments, the suitable conditions used to prepare a compound of formula 22 from a compound of formula 20c include conditions known in the art to remove PG¹ and PG², and promote hydrolysis of the boroxine ring and intramolecular cyclization by a condensation reaction (e.g., removal of water). In some embodiments, the conditions include acidic conditions, basic conditions, reducing conditions, thermal conditions, or combinations thereof. In some embodiments, PG¹ and PG² are removed under acidic conditions (e.g., using mineral acids, organic acids, etc.). In some embodiments, PG¹ and PG² are removed under basic conditions (e.g., using hydroxides, organic bases, etc.). In some embodiments, PG¹ and PG² are removed under reducing conditions (e.g., using hydride reagents, hydrogenolysis with compositions comprising palladium, nickel, or platinum, etc.). In some embodiments, PG¹ and PG² are removed under thermal conditions. In some embodiments, removal of PG¹ and PG², hydrolysis of the boroxine ring, and intramolecular cyclization are promoted under the same conditions. In some embodiments, removal of PG¹ and PG², hydrolysis of the boroxine ring, and intramolecular cyclization are promoted under the different conditions. In some embodiments, intramolecular cyclization is promoted by azeotropic distillation. In some embodiments, intramolecular cyclization is promoted by slurrying in a hot alcohol.

In certain embodiments, removal of PG¹ and PG² and hydrolysis of the boroxine ring occurs under reducing conditions (e.g., hydrogenolysis with a palladium composition) and intramolecular cyclization is facilitated by azeotropic distillation in an alcohol (e.g., methanol, ethanol, isopropanol, and the like).

In certain embodiments, removal of PG¹ and PG² occurs under reducing conditions (e.g., hydrogenolysis with a palladium composition) and hydrolysis of the boroxine ring and intramolecular cyclization is facilitated by azeotropic distillation in an alcohol (e.g., methanol, ethanol, isopropanol, and the like).

In some embodiments, a process to prepare a compound of formula 22 from a compound of formula 20c includes providing a compound of formula 20c in a crude form or a purified form. In certain embodiments, a compound of formula 20c is provided in a purified form after a crystallization step. In some embodiments, a compound of formula 20c is provided in greater than 90%, 95%, 96%, 97%, or greater than 98% purity. In some embodiments, a compound of formula 20c is isolated as a solid appropriate for use as an intermediate compound. For instance, in one embodiment, a compound of formula 20c is isolated as a solid appropriate for use in producing a compound of formula 21 or formula 22. In one such embodiment, a compound of formula 20c is a crystalline solid for use in producing a compound of formula 21 or formula 22.

In certain embodiments, the process comprises preparing a compound of formula 22, wherein the compound of formula 20c is:

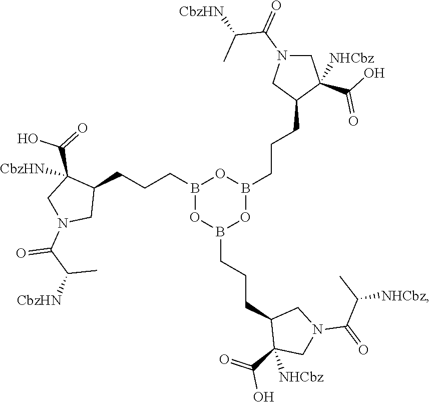 [SMILES: C[C@H](NC(=O)OCc1ccccc1)C(=O)N1C[C@H](CCCB2OB(CCC[C@H]3CN(C(=O)[C@H](C)NC(=O)OCc4ccccc4)C[C@@]3(NC(=O)OCc3ccccc3)C(=O)O)OB(CCC[C@H]3CN(C(=O)[C@H](C)NC(=O)OCc4ccccc4)C[C@@]3(NC(=O)OCc3ccccc3)C(=O)O)O2)[C@](NC(=O)OCc2ccccc2)(C(=O)O)C1]

(3R,3′R,3″R,4S,4′S,4″S)-4,4′,4″-((1,3,5,2,4,6-trioxatriborinane-2,4,6-triyl)tris(propane-3,1-diyl))tris(1-(((benzyloxy)carbonyl)-L-alanyl)-3-(((benzyloxy)carbonyl)amino)pyrrolidine-3-carboxylic acid) or salt thereof.

In certain embodiments, the process comprises preparing a compound of formula 22, wherein the compound of formula 20c is:

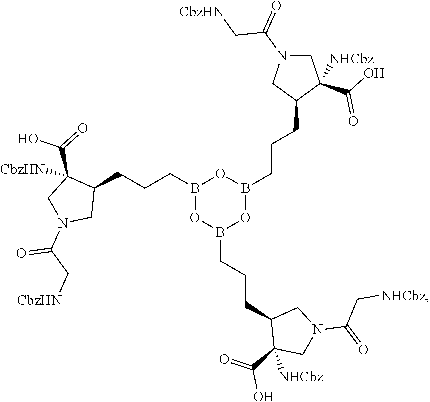 [SMILES: O=C(NCC(=O)N1C[C@H](CCCB2OB(CCC[C@H]3CN(C(=O)CNC(=O)OCc4ccccc4)C[C@@]3(NC(=O)OCc3ccccc3)C(=O)O)OB(CCC[C@H]3CN(C(=O)CNC(=O)OCc4ccccc4)C[C@@]3(NC(=O)OCc3ccccc3)C(=O)O)O2)[C@](NC(=O)OCc2ccccc2)(C(=O)O)C1)OCc1ccccc1]

(3R,3′R,3″R,4S,4′S,4″S)-4,4′,4″-((1,3,5,2,4,6-trioxatriborinane-2,4,6-triyl)tris(propane-3,1-diyl))tris(3-(((benzyloxy)carbonyl)amino)-1-(((benzyloxy)carbonyl)glycyl)pyrrolidine-3-carboxylic acid) or salt thereof.

In some embodiments, the alternative process for preparing a compound of formula 22 further comprises preparing the compound of formula 20c, the process comprising treating a compound of formula 21c

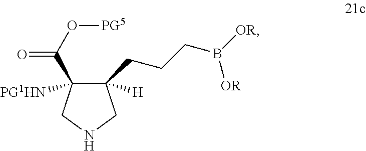

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle; and
PG¹ is a suitable amine protecting group;
PG⁵ is a suitable carboxylic acid protecting group,
   under suitable conditions to form the compound of formula 20c or a salt thereof.

In some embodiments, the suitable conditions used to prepare a compound of formula 20c from a compound of formula 21c include conditions known in the art to a) form an amide bond between a secondary amine and a carboxylic acid; b) hydrolyze a pinacol ester boronate to a boronic acid; c) deprotect a primary amine (e.g., PG¹ is —COCF₃) and carboxylic acid protecting group (e.g., PG⁵ is C₁₋₆ aliphatic); d) add a protecting group (e.g., PG¹) to a primary amine; and e) dehydrate boronic acids to form boroxines.

In some embodiments, the suitable conditions to form an amide bond include amide forming conditions using an amide coupling reagent known in the art such as, but not limited to HATU, PyBOP, DCC, DIC, EDC, HBTU, HCTU, PyAOP, PyBrOP, BOP, BOP-Cl, DEPBT, T₃P, TATU, TBTU, TNTU, TOTU, TPTU, TSTU, or TDBTU. In certain embodiments, the amide coupling reagent is 1-propanephosphonic anhydride solution (T₃P).

In some embodiments, the suitable conditions to hydrolyze a pinacol ester boronate to a boronic acid include oxidative cleavage with sodium periodate, biphasic transesterification with other boronic acids, transborylation with boron trichloride, acidic hydrolysis, solid-phase processes using polystyrene-boronic acid, formation of fluorinated intermediates followed by trimethylsilyl chloride or lithium hydroxide, or transesterification with diethanolamine followed by acidic hydrolysis. In certain embodiments, the pinacol ester boronate is hydrolyzed by transesterification with phenylboronic acid under acidic conditions (e.g., using mineral acids, organic acids, etc.).

In some embodiments, the suitable conditions used to deprotect a primary amine (e.g., PG¹ is —COCF₃) and carboxylic acid protecting group (e.g., PG⁵ is C₁₋₆ aliphatic) include basic conditions (e.g., using hydroxides, organic bases, etc.) or acidic conditions (e.g., using mineral acids, organic acids, etc.). In certain embodiments, the suitable conditions include the use of lithium hydroxide.

In some embodiments, the suitable conditions to add a protecting group to a primary amine includes the use of suitable amine protecting groups and the reagents and reaction conditions appropriate for using them include those defined above and described herein. In certain embodiments, PG¹ is benzyloxycarbonyl (Cbz) added using benzyl (2,5-dioxopyrrolidin-1-yl) carbonate (Z-OSu) and a base (e.g., a carbonate or amine base).

In some embodiments, the conditions used to dehydrate boronic acids to form boroxines include using a drying agent (e.g., sulfuric acid, phosphorus pentoxide, etc.), heating under a high vacuum, azeotropic distillation, or warming in an anhydrous solvent (e.g., carbon tetrachloride or chloroform). In certain embodiments, the conditions include azeotropic distillation in isopropyl acetate.

In some embodiments, a process to prepare a compound of formula 20c from a compound of formula 21c includes providing a compound of formula 21c in a crude form or a purified form. In certain embodiments, a compound of formula 21c is provided in a purified form after a crystallization step. In some embodiments, a compound of formula 21c is provided in greater than 90%, 91%, 92%, 93%, or greater than 94% purity. In some embodiments, a compound of formula 21c is isolated as a solid appropriate for use as an intermediate compound. For instance, in one embodiment, a compound of formula 21c is isolated as a solid appropriate for use in producing a compound of formula 21 or formula 22. In one such embodiment, a compound of formula 21c is a crystalline solid for use in producing a compound of formula 21 or formula 22.

In certain embodiments, the alternate process comprises preparing a compound of formula 21c, wherein the compound is:

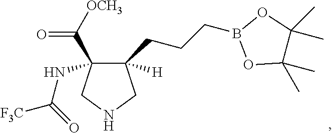 [SMILES: COC(=O)[C@]1(NC(=O)C(F)(F)F)CNC[C@@H]1CCCB1OC(C)(C)C(C)(C)O1]

methyl (3R,4S)-4-(3-(4,4,5,5-tetramethyl-1,3,2-dioxaborolan-2-yl)propyl)-3-(2,2,2-trifluoroacetamido)pyrrolidine-3-carboxylate or salt thereof.

In some embodiments, the alternative process for preparing a compound of formula 22 further comprises preparing the compound of formula 21c, the process comprising removing a protecting group to the primary amine and carboxylic acid of a compound of formula 16c

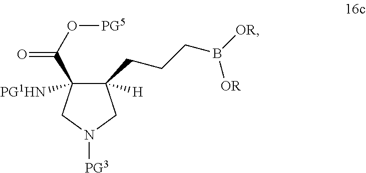

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
PG¹ and PG³ are independently suitable amine protecting groups; and
PG⁵ is a suitable carboxylic acid protecting group,
   under suitable conditions to form the compound of formula 21c or a salt thereof.

Suitable amine protecting groups and the reagents and reaction conditions appropriate for using them to protect and deprotect amine groups include those defined above and described herein. In some embodiments, PG¹ is benzyloxycarbonyl (Cbz). In some embodiments, PG³ is tert-butyloxycarbonyl (Boc).

Suitable carboxylic acid protecting groups and the reagents and reaction conditions appropriate for using them to protect and deprotect carboxylic acid groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Representative carboxylic acid protecting groups include, but are not limited to, optionally substituted C₁₋₆ aliphatic esters, optionally substituted aryl esters, optionally substituted benzyl esters, silyl esters, dihydroxazoles, activated esters (e.g., derivatives of nitrophenol, pentafluorophenol, N-hydroxylsuccinimide, hydroxybenzotriazole, etc.), orthoesters, and the like. In some embodiments, PG⁵ is methyl. In some embodiments, PG⁵ is ethyl. In some embodiments, PG⁵ is isopropyl. In some embodiments, PG⁵ is tert-butyl.

In some embodiments, a process to prepare a compound of formula 21c from a compound of formula 16c includes providing a compound of formula 16c in a crude form or a purified form. In certain embodiments, the compound of formula 16c is provided in a crude form.

In some embodiments, the alternative process for preparing a compound of formula 22 further comprises preparing the compound of formula 16c, the process comprising hydroborating a compound of formula 15c

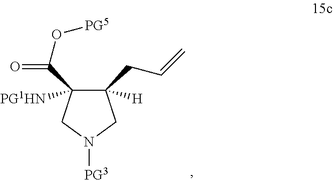

or a salt thereof, wherein:
PG¹ and PG³ are independently suitable amine protecting groups;
PG⁵ is a suitable carboxylic acid protecting group,
   under suitable conditions to form the compound of formula 16c or a salt thereof.

In some embodiments, the suitable conditions used to prepare a compound of formula 16c from a compound of formula 15c include conditions known in the art to hydroborate an alkene, specifically a terminal alkene. In some embodiments, the conditions include hydroboration using a borane reagent or metal-catalyzed hydroboration using pinacolborane or bis(pinacolato)diboron (B₂Pin₂) and a metal catalyst comprising rhodium, iridium, iron, and the like. In certain embodiments, the suitable conditions include hydroboration using an iridium catalyst and pinacolborane.

In certain embodiments, the alternate process comprises preparing a compound of formula 15c, wherein the compound is:

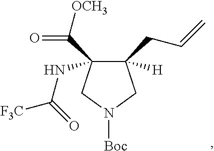 [SMILES: C=CC[C@H]1CN(C(=O)OC(C)(C)C)C[C@@]1(NC(=O)C(F)(F)F)C(=O)OC]

1-(tert-butyl) 3-methyl (3R,4S)-4-allyl-3-(2,2,2-trifluoroacetamido)pyrrolidine-1,3-dicarboxylate or salt thereof.

3.5.2. Alternate Route B

In some embodiments, a compound of formula 22 is prepared according to Scheme 5.

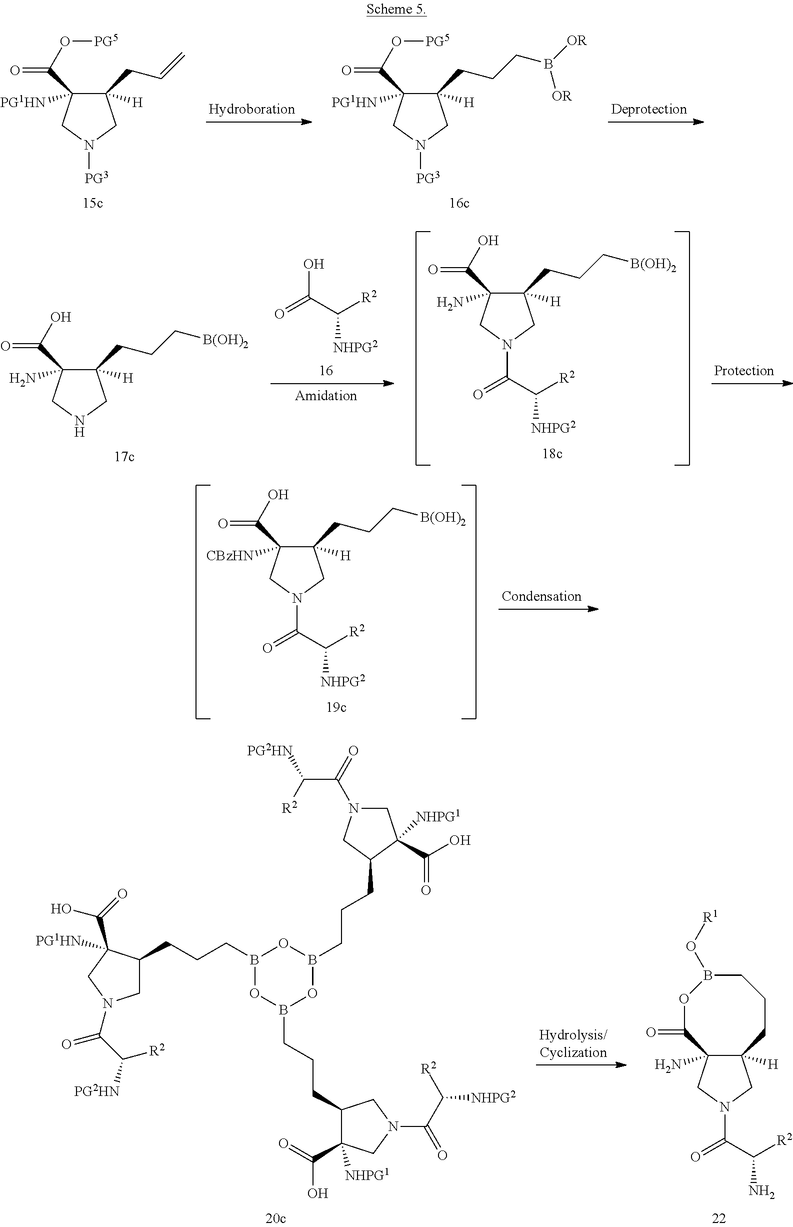

In Scheme 5 above, the reactions steps and variables R, R¹, R², PG¹, PG², PG³, and PG⁵ are defined above and described herein.

In some embodiments, the alternate process of preparing a compound of formula 22 or a pharmaceutically acceptable salt thereof comprises treating a compound of formula 20c

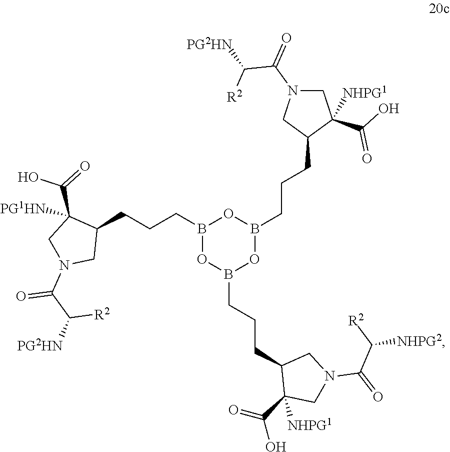

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

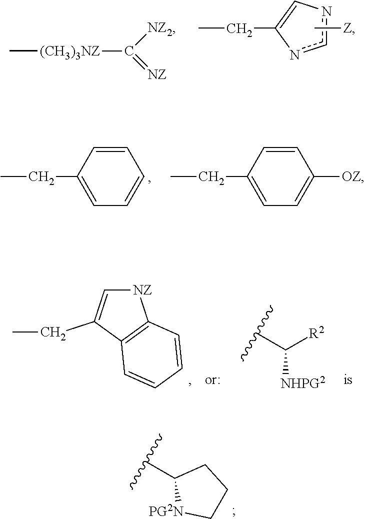

each Z is independently H, (C₁-C₆)alkyl, or a suitable protecting group; and
PG¹ and PG² are independently suitable amine protecting groups,
   under suitable conditions to form the compound of formula 22 or a pharmaceutically acceptable salt thereof.

In some embodiments, the suitable conditions used to prepare a compound of formula 22 from a compound of formula 20c include conditions known in the art to remove PG¹ and PG², and promote hydrolysis of the boroxine ring and intramolecular cyclization by a condensation reaction (e.g., removal of water). In some embodiments, the conditions include acidic conditions, basic conditions, reducing conditions, thermal conditions, or combinations thereof. In some embodiments, PG¹ and PG² are removed under acidic conditions (e.g., using mineral acids, organic acids, etc.). In some embodiments, PG¹ and PG² are removed under basic conditions (e.g., using hydroxides, organic bases, etc.). In some embodiments, PG¹ and PG² are removed under reducing conditions (e.g., using hydride reagents, hydrogenolysis with compositions comprising palladium, nickel, or platinum, etc.). In some embodiments, PG¹ and PG² are removed under thermal conditions. In some embodiments, removal of PG¹ and PG², hydrolysis of the boroxine ring, and intramolecular cyclization are promoted under the same conditions. In some embodiments, removal of PG¹ and PG², hydrolysis of the boroxine ring, and intramolecular cyclization are promoted under the different conditions. In some embodiments, intramolecular cyclization is promoted by azeotropic distillation. In some embodiments, intramolecular cyclization is promoted by slurrying in a hot alcohol.

In certain embodiments, removal of PG¹ and PG² and hydrolysis of the boroxine ring occurs under reducing conditions (e.g., hydrogenolysis with a palladium composition) and intramolecular cyclization is facilitated by azeotropic distillation in an alcohol (e.g., methanol, ethanol, isopropanol, and the like).

In certain embodiments, removal of PG¹ and PG² occurs under reducing conditions (e.g., hydrogenolysis with a palladium composition) and hydrolysis of the boroxine ring and intramolecular cyclization is facilitated by azeotropic distillation in an alcohol (e.g., methanol, ethanol, isopropanol, and the like).

In some embodiments, a process to prepare a compound of formula 22 from a compound of formula 20c includes providing a compound of formula 20c in a crude form or a purified form. In certain embodiments, a compound of formula 20c is provided in a purified form after a crystallization step. In some embodiments, a compound of formula 20c is provided in greater than 90%, 95%, 96%, 97%, or greater than 98% purity. In some embodiments, a compound of formula 20c is isolated as a solid appropriate for use as an intermediate compound. For instance, in one embodiment, a compound of formula 20c is isolated as a solid appropriate for use in producing a compound of formula 21 or formula 22. In one such embodiment, a compound of formula 20c is a crystalline solid for use in producing a compound of formula 21 or formula 22.

In some embodiments, the alternative process for preparing a compound of formula 22 further comprises preparing the compound of formula 20c, the process comprising treating a compound of formula 19c

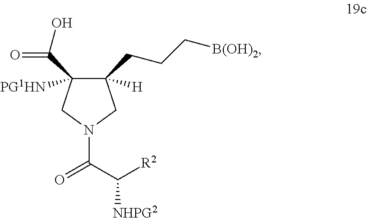

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

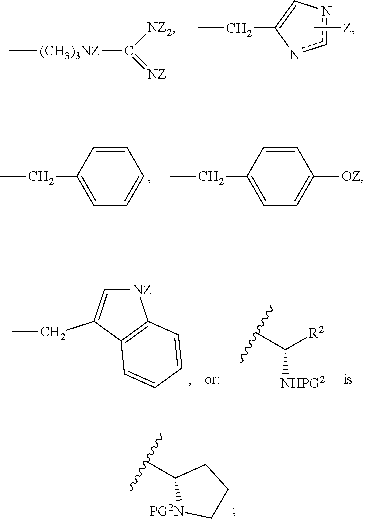

each Z is independently H, (C₁-C₆)alkyl, or a suitable protecting group; and
PG¹ and PG² are independently suitable amine protecting groups,
   under suitable conditions to form the compound of formula 20c or a salt thereof.

In some embodiments, the suitable conditions used to prepare a compound of formula 20c from a compound of formula 19c include conditions known in the art to dehydrate boronic acids to form boroxines. In some embodiments, the conditions include using a drying agent (e.g., sulfuric acid, phosphorus pentoxide, etc.), heating under a high vacuum, azeotropic distillation, or warming in an anhydrous solvent (e.g., carbon tetrachloride or chloroform). In certain embodiments, the conditions include azeotropic distillation in isopropyl acetate.

In some embodiments, a process to prepare a compound of formula 20c from a compound of formula 19c includes providing a compound of formula 19c in a crude form or a purified form. In certain embodiments, a compound of formula 19c is provided in a crude form after one or more extraction and washing steps.

In certain embodiments, the process comprises preparing a compound of formula 20c, wherein the compound of formula 19c is:

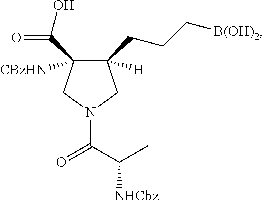

(3R,4S)-1-(((benzyloxy)carbonyl)-L-alanyl)-4-(3-boronopropyl)-3-((2-oxo-2-phenyl-1λ²-ethyl)amino)pyrrolidine-3-carboxylic acid or salt thereof.

In some embodiments, the alternative process for preparing a compound of formula 22 further comprises preparing the compound of formula 19c, the process comprising adding a protecting group to the primary amine of a compound of formula 18c

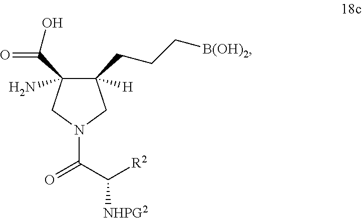

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

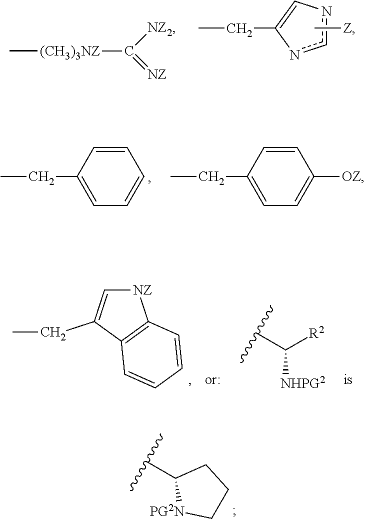

each Z is independently H, (C₁-C₆)alkyl, or a suitable protecting group; and
PG² is a suitable amine protecting group,
   under suitable conditions to form the compound of formula 19c or a salt thereof.

In some embodiments, the suitable conditions used to prepare a compound of formula 19c from a compound of formula 18c include conditions known in the art for adding a protecting group (e.g., PG¹) to a primary amine. Suitable amine protecting groups and the reagents and reaction conditions appropriate for using them to protect and deprotect amine groups include those defined above and described herein. In certain embodiments, PG¹ is benzyloxycarbonyl (Cbz) added using benzyl (2,5-dioxopyrrolidin-1-yl) carbonate (Z-OSu) and abase (e.g., a carbonate or amine base).

In some embodiments, a process to prepare a compound of formula 19c from a compound of formula 18c includes providing a compound of formula 18c in a crude form or a purified form. In certain embodiments, a compound of formula 18c is provided in a crude form after one or more extraction and washing steps.

In certain embodiments, the process comprises preparing a compound of formula 19c, wherein the compound of formula 18c is:

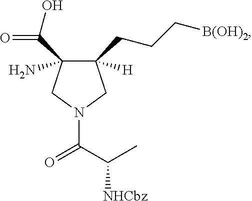 [SMILES: C[C@H](NC(=O)OCc1ccccc1)C(=O)N1C[C@H](CCCB(O)O)[C@](N)(C(=O)O)C1]

(3R,4S)-3-amino-1-(((benzyloxy)carbonyl)-L-alanyl)-4-(3-boronopropyl)pyrrolidine-3-carboxylic acid or salt thereof.

In some embodiments, the alternate process for preparing a compound of formula 22 further comprises preparing the compound of formula 18c, the process comprising coupling a compound of formula 17c

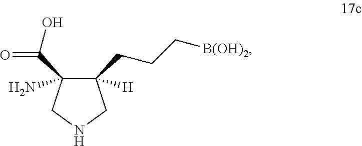 [SMILES: N[C@@]1(C(=O)O)CNC[C@@H]1CCCB(O)O]

or a salt thereof,
with a compound of formula 16

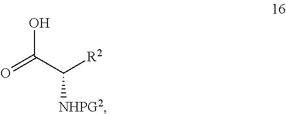

or salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

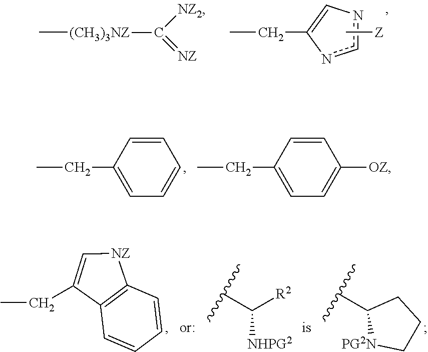

each Z is independently H, (C₁-C₆)alkyl, or a suitable protecting group; and
PG² is a suitable amine protecting group,
   under amide forming conditions to form the compound of formula 18c or a salt thereof.

In some embodiments, the amide forming conditions used to prepare a compound of formula 18c from a compound of formula 17c and a compound of formula 16 include conditions known in the art to form an amide bond between a secondary amine and a carboxylic acid. In some embodiments, the amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T₃P, TATU, TBTU, TNTU, TOTU, TPTU, TSTU, or TDBTU. In certain embodiments, the amide coupling reagent is 1-propanephosphonic anhydride solution (T₃P).

In some embodiments, a process to prepare a compound of formula 18c from a compound of formula 17c includes providing a compound of formula 17c in a crude form or a purified form. In certain embodiments, a compound of formula 17c is provided in a purified form after a crystallization step. In some embodiments, a compound of formula 17c is provided in greater than 90%, 91%, 92%, 93%, or greater than 94% purity. In some embodiments, a compound of formula 17c is isolated as a solid appropriate for use as an intermediate compound. For instance, in one embodiment, a compound of formula 17c is isolated as a solid appropriate for use in producing a compound of formula 21 or formula 22. In one such embodiment, a compound of formula 17c is a crystalline solid for use in producing a compound of formula 21 or formula 22.

In some embodiments, the alternative process for preparing a compound of formula 22 further comprises preparing the compound of formula 17c, the process comprising adding a protecting group to the primary amine of a compound of formula 16c

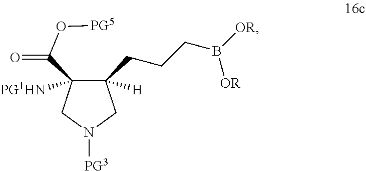

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
PG¹ and PG³ are independently suitable amine protecting groups; and
PG⁵ is a suitable carboxylic acid protecting group,
   under suitable conditions to form the compound of formula 17c or a salt thereof.

Suitable amine protecting groups and the reagents and reaction conditions appropriate for using them to protect and deprotect amine groups include those defined above and described herein. In some embodiments, PG¹ is benzyloxycarbonyl (Cbz). In some embodiments, PG³ is tert-butyloxycarbonyl (Boc).

Suitable carboxylic acid protecting groups and the reagents and reaction conditions appropriate for using them to protect and deprotect carboxylic acid groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Representative carboxylic acid protecting groups include, but are not limited to, optionally substituted C₁₋₆ aliphatic esters, optionally substituted aryl esters, optionally substituted benzyl esters, silyl esters, dihydroxazoles, activated esters (e.g., derivatives of nitrophenol, pentafluorophenol, N-hydroxylsuccinimide, hydroxybenzotriazole, etc.), orthoesters, and the like. In some embodiments, PG⁵ is methyl. In some embodiments, PG⁵ is ethyl. In some embodiments, PG⁵ is isopropyl. In some embodiments, PG⁵ is tert-butyl.

In some embodiments, the suitable conditions used to prepare a compound of formula 17c from a compound of formula 16c include conditions known in the art to remove a primary amine protecting group, remove a carboxylic acid protecting group, and hydrolyze a pinacol ester boronate to a boronic acid.

In some embodiments, the suitable conditions to hydrolyze a pinacol ester boronate include oxidative cleavage with sodium periodate, biphasic transesterification with other boronic acids, transborylation with boron trichloride, acidic hydrolysis, solid-phase processes using polystyrene-boronic acid, formation of fluorinated intermediates followed by trimethylsilyl chloride or lithium hydroxide, or transesterification with diethanolamine followed by acidic hydrolysis.

In certain embodiments, the conditions to remove a primary amine protecting group, remove a carboxylic acid protecting group, and hydrolyze a pinacol ester boronate of a compound of formula 16c is achieved using acidic hydrolysis (e.g., a mineral acid such as HCl).

In some embodiments, a process to prepare a compound of formula 17c from a compound of formula 16c includes providing a compound of formula 16c in a crude form or a purified form.

In certain embodiments, the process comprises preparing a compound of formula 17c, wherein the compound of formula 16c is:

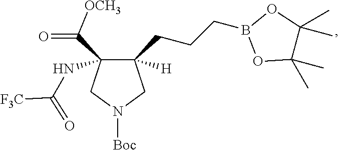 [SMILES: COC(=O)[C@]1(NC(=O)C(F)(F)F)CN(C(=O)OC(C)(C)C)C[C@@H]1CCCB1OC(C)(C)C(C)(C)O1]

1-(tert-butyl) 3-methyl (3R,4S)-4-(3-(4,4,5,5-tetramethyl-1,3,2-dioxaborolan-2-yl)propyl)-3-(2,2,2-trifluoroacetamido)pyrrolidine-1,3-dicarboxylate or salt thereof.

In some embodiments, the alternative process for preparing a compound of formula 22 further comprises preparing the compound of formula 16c, the process comprising hydroborating a compound of formula 15c

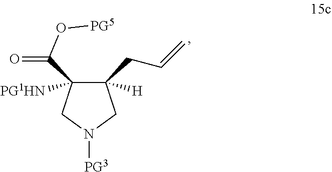

or a salt thereof, wherein:
PG¹ and PG³ are independently suitable amine protecting groups;
PG⁵ is a suitable carboxylic acid protecting group,
   under suitable conditions to form the compound of formula 15c or a salt thereof.

In some embodiments, the suitable conditions used to prepare a compound of formula 16c from a compound of formula 15c include conditions known in the art to hydroborate an alkene, specifically a terminal alkene. In some embodiments, the conditions include hydroboration using a borane reagent or metal-catalyzed hydroboration using pinacolborane or bis(pinacolato)diboron (B₂Pin₂) and a metal catalyst comprising rhodium, iridium, iron, and the like. In certain embodiments, the suitable conditions include hydroboration using an iridium catalyst and pinacolborane.

### 3.6. Compounds of the Disclosure

Also provided herein are intermediate compounds useful for the preparation of a compound of formula 21 or formula 22.

In some embodiments, the present disclosure provides a compound of formula 20:

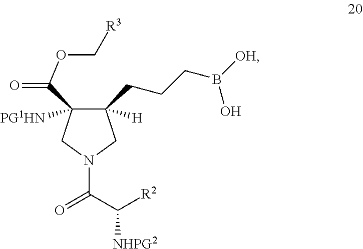

or a salt thereof, wherein:
R² is H, (C₁-C₆)alkyl, —CH₂OZ, —CH(CH₃)OZ, —CH₂SZ, —(CH₂)₂SCH₃—CH₂CONZ₂, —(CH₂)₂CONZ₂, —CH₂CO₂Z, —(CH₂)₂CO₂Z, —(CH₂)₄NZ₂,

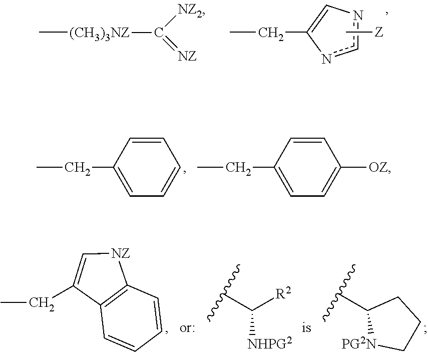

each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

In certain embodiments, the compound of formula 20 is:

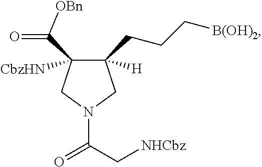 [SMILES: O=C(NCC(=O)N1C[C@H](CCCB(O)O)[C@](NC(=O)OCc2ccccc2)(C(=O)OCc2ccccc2)C1)OCc1ccccc1]

(3-((3S,4R)-4-((benzyloxy)carbonyl)-4-(((benzyloxy)carbonyl)amino)-1-(((benzyloxy)carbonyl)glycyl)pyrrolidin-3-yl)propyl)boronic acid or salt thereof.

In certain embodiments, the compound of formula 20 is:

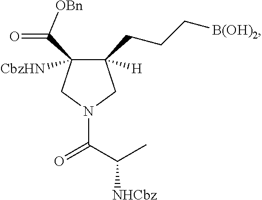 [SMILES: C[C@H](NC(=O)OCc1ccccc1)C(=O)N1C[C@H](CCCB(O)O)[C@](NC(=O)OCc2ccccc2)(C(=O)OCc2ccccc2)C1]

(3-((3S,4R)-4-((benzyloxy)carbonyl)-1-(((benzyloxy)carbonyl)-L-alanyl)-4-(((benzyloxy)carbonyl)amino)pyrrolidin-3-yl)propyl)boronic acid or salt thereof.

In certain embodiments, the compound of formula 20 is:

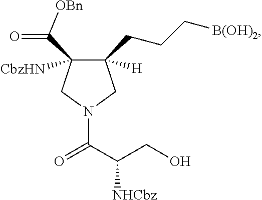 [SMILES: O=C(N[C@@H](CO)C(=O)N1C[C@H](CCCB(O)O)[C@](NC(=O)OCc2ccccc2)(C(=O)OCc2ccccc2)C1)OCc1ccccc1]

(3-((3S,4R)-4-((benzyloxy)carbonyl)-1-(((benzyloxy)carbonyl)-L-seryl)-4-(((benzyloxy)carbonyl)amino)pyrrolidin-3-yl)propyl)boronic acid or salt thereof.

In some embodiments, the present disclosure provides a compound of formula 19:

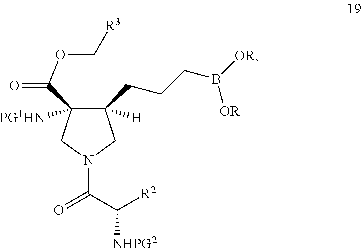

or a salt thereof, wherein:
each R is independently (C₁-C₆)alkyl, or:
   two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

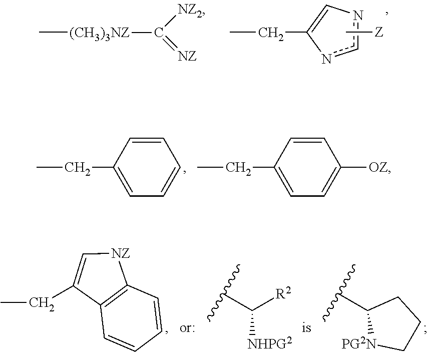

each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

In certain embodiments of the compound of formula 19, each R is independently selected from methyl, ethyl, and isopropyl.

In certain embodiments of the compound of formula 19, the two R groups are taken together with their intervening atoms to form a substituted or unsubstituted monocyclic or bicyclic ring selected from

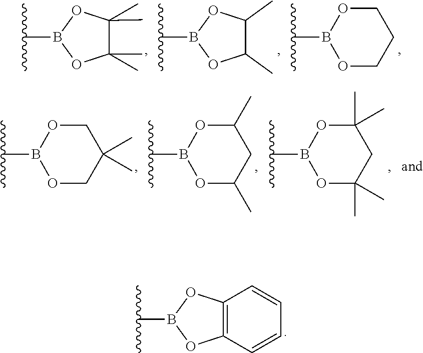

In certain embodiments, the compound of formula 19 is:

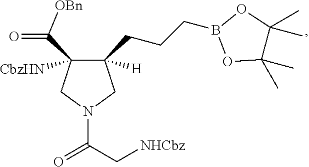 [SMILES: CC1(C)OB(CCC[C@H]2CN(C(=O)CNC(=O)OCc3ccccc3)C[C@@]2(NC(=O)OCc2ccccc2)C(=O)OCc2ccccc2)OC1(C)C]

benzyl (3R,4S)-3-(((benzyloxy)carbonyl)amino)-1-(((benzyloxy)carbonyl)glycyl)-4-(3-(4,4,5,5-tetramethyl-1,3,2-dioxaborolan-2-yl)propyl)pyrrolidine-3-carboxylate or salt thereof.

In certain embodiments, the compound of formula 19 is:

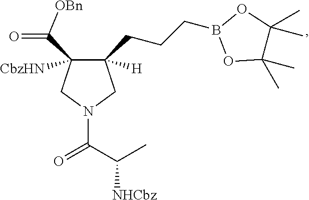 [SMILES: C[C@H](NC(=O)OCc1ccccc1)C(=O)N1C[C@H](CCCB2OC(C)(C)C(C)(C)O2)[C@](NC(=O)OCc2ccccc2)(C(=O)OCc2ccccc2)C1]

benzyl (3R,4S)-1-(((benzyloxy)carbonyl)-L-alanyl)-3-(((benzyloxy)carbonyl)amino)-4-(3-(4,4,5,5-tetramethyl-1,3,2-dioxaborolan-2-yl)propyl)pyrrolidine-3-carboxylate or salt thereof.

In certain embodiments, the compound of formula 19 is:

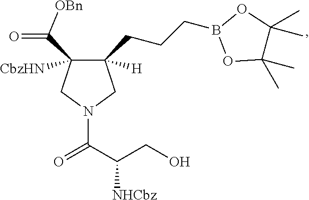 [SMILES: CC1(C)OB(CCC[C@H]2CN(C(=O)[C@H](CO)NC(=O)OCc3ccccc3)C[C@@]2(NC(=O)OCc2ccccc2)C(=O)OCc2ccccc2)OC1(C)C]

benzyl (3R,4S)-1-(((benzyloxy)carbonyl)-L-seryl)-3-(((benzyloxy)carbonyl)amino)-4-(3-(4,4,5,5-tetramethyl-1,3,2-dioxaborolan-2-yl)propyl)pyrrolidine-3-carboxylate or salt thereof.

In some embodiments, the present disclosure provides a compound of formula 18:

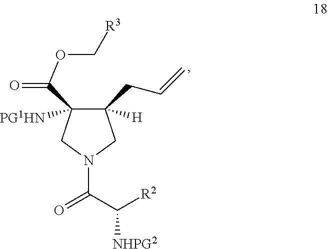

or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂,

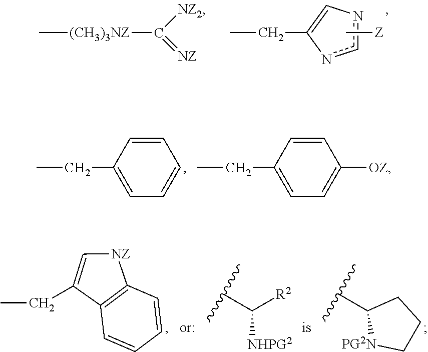

each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

In certain embodiments, the compound of formula 18 is:

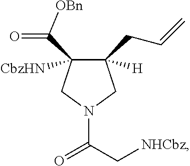 [SMILES: C=CC[C@H]1CN(C(=O)CNC(=O)OCc2ccccc2)C[C@@]1(NC(=O)OCc1ccccc1)C(=O)OCc1ccccc1]

benzyl (3R,4S)-4-allyl-3-(((benzyloxy)carbonyl)amino)-1-(((benzyloxy)carbonyl)glycyl)pyrrolidine-3-carboxylate or salt thereof.

In certain embodiments, the compound of formula 18 is:

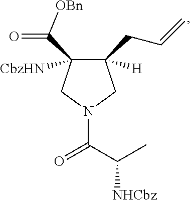 [SMILES: C=CC[C@H]1CN(C(=O)[C@H](C)NC(=O)OCc2ccccc2)C[C@@]1(NC(=O)OCc1ccccc1)C(=O)OCc1ccccc1]

benzyl (3R,4S)-4-allyl-1-(((benzyloxy)carbonyl)-L-alanyl)-3-(((benzyloxy)carbonyl)amino)pyrrolidine-3-carboxylate or salt thereof.

In certain embodiments, the compound of formula 18 is:

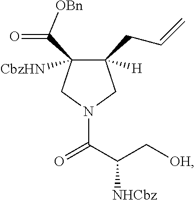 [SMILES: C=CC[C@H]1CN(C(=O)[C@H](CO)NC(=O)OCc2ccccc2)C[C@@]1(NC(=O)OCc1ccccc1)C(=O)OCc1ccccc1]

benzyl (3R,4S)-4-allyl-1-(((benzyloxy)carbonyl)-L-seryl)-3-(((benzyloxy)carbonyl)amino)pyrrolidine-3-carboxylate or salt thereof.

In certain embodiments, the compound of formula 18 is:

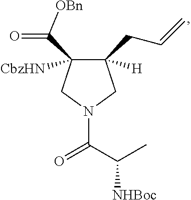 [SMILES: C=CC[C@H]1CN(C(=O)[C@H](C)NC(=O)OC(C)(C)C)C[C@@]1(NC(=O)OCc1ccccc1)C(=O)OCc1ccccc1]

benzyl (3R,4S)-4-allyl-3-(((benzyloxy)carbonyl)amino)-1-((tert-butoxycarbonyl)-L-alanyl)pyrrolidine-3-carboxylate or salt thereof.

In certain embodiments, the compound of formula 18 is:

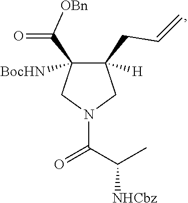 [SMILES: C=CC[C@H]1CN(C(=O)[C@H](C)NC(=O)OCc2ccccc2)C[C@@]1(NC(=O)OC(C)(C)C)C(=O)OCc1ccccc1]

benzyl (3R,4S)-4-allyl-1-(((benzyloxy)carbonyl)-L-alanyl)-3-((tert-butoxycarbonyl)amino)pyrrolidine-3-carboxylate or salt thereof.

In certain embodiments, the compound of formula 18 is:

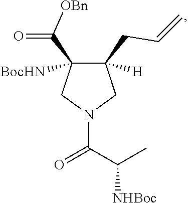 [SMILES: C=CC[C@H]1CN(C(=O)[C@H](C)NC(=O)OC(C)(C)C)C[C@@]1(NC(=O)OC(C)(C)C)C(=O)OCc1ccccc1]

benzyl (3R,4S)-4-allyl-1-((tert-butoxycarbonyl)-L-alanyl)-3-((tert-butoxycarbonyl)amino)pyrrolidine-3-carboxylate or salt thereof.

In certain embodiments, the compound of formula 18 is:

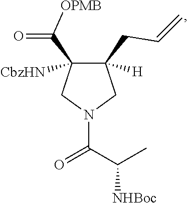

4-methoxybenzyl (3R,4S)-4-allyl-3-(((benzyloxy)carbonyl)amino)-1-((tert-butoxycarbonyl)-L-alanyl)pyrrolidine-3-carboxylate or salt thereof.

In certain embodiments, the compound of formula 18 is:

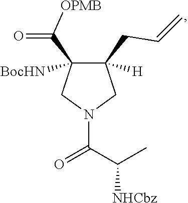

4-methoxybenzyl (3R,4S)-4-allyl-1-(((benzyloxy)carbonyl)-L-alanyl)-3-((tert-butoxycarbonyl)amino)pyrrolidine-3-carboxylate or salt thereof.

In certain embodiments, the compound of formula 18 is:

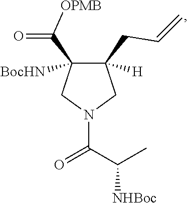

4-methoxybenzyl (3R,4S)-4-allyl-1-((tert-butoxycarbonyl)-L-alanyl)-3-((tert-butoxycarbonyl)amino)pyrrolidine-3-carboxylate or salt thereof.

In some embodiments, the present disclosure provides a compound of formula 17:

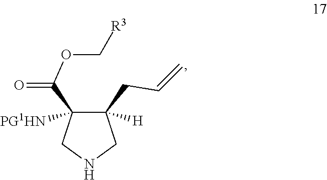

or a salt thereof, wherein:
R³ is a substituted or unsubstituted ring selected from aryl and heteroaryl; and
PG¹ is a suitable amine protecting group.

In certain embodiments, the compound of formula 17 is:

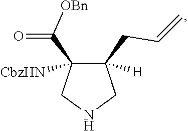 [SMILES: C=CC[C@H]1CNC[C@@]1(NC(=O)OCc1ccccc1)C(=O)OCc1ccccc1]

benzyl (3R,4S)-4-allyl-3-(((benzyloxy)carbonyl)amino)pyrrolidine-3-carboxylate or salt thereof.

In some embodiments, the present disclosure provides a compound of formula 15:

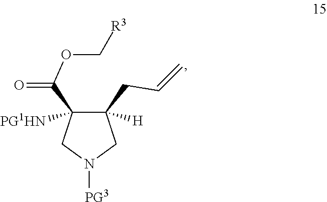

or a salt thereof, wherein:
R³ is a substituted or unsubstituted ring selected from aryl and heteroaryl; and
PG¹ and PG³ are independently suitable amine protecting groups.

In certain embodiments, a compound of formula 15 is:

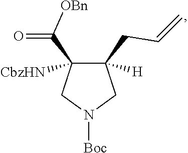 [SMILES: C=CC[C@H]1CN(C(=O)OC(C)(C)C)C[C@@]1(NC(=O)OCc1ccccc1)C(=O)OCc1ccccc1]

3-benzyl 1-(tert-butyl) (3R,4S)-4-allyl-3-(((benzyloxy)carbonyl)amino)pyrrolidine-1,3-dicarboxylate or salt thereof.

In some embodiments, the present disclosure provides a compound of formula 14:

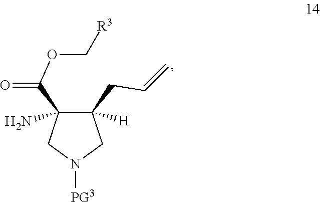

or a salt thereof, wherein:
R³ is a substituted or unsubstituted ring selected from aryl and heteroaryl; and
PG³ is a suitable amine protecting group.

In certain embodiments, the compound of formula 14 is:

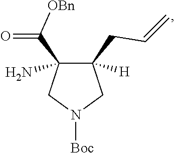 [SMILES: C=CC[C@H]1CN(C(=O)OC(C)(C)C)C[C@@]1(N)C(=O)OCc1ccccc1]

3-benzyl 1-(tert-butyl) (3R,4S)-4-allyl-3-aminopyrrolidine-1,3-dicarboxylate or salt thereof.

In some embodiments, the present disclosure provides a compound of formula 13:

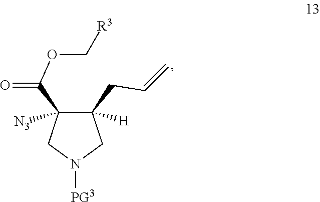

or a salt thereof, wherein:
R³ is a substituted or unsubstituted ring selected from aryl and heteroaryl; and
PG³ is a suitable amine protecting group.

In certain embodiments, the compound of formula 13 is:

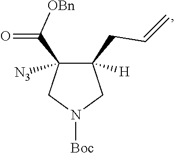 [SMILES: C=CC[C@H]1CN(C(=O)OC(C)(C)C)C[C@@]1(N=[N+]=[N-])C(=O)OCc1ccccc1]

3-benzyl 1-(tert-butyl) (3R,4S)-4-allyl-3-azidopyrrolidine-1,3-dicarboxylate or salt thereof.

In some embodiments, the present disclosure provides a compound of formula 12:

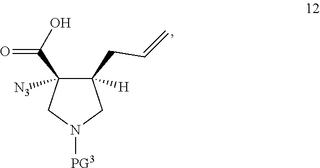

or a salt thereof, wherein:
PG³ is a suitable amine protecting group.

In certain embodiments, the compound of formula 12 is:

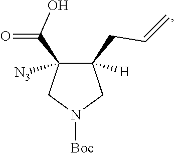 [SMILES: C=CC[C@H]1CN(C(=O)OC(C)(C)C)C[C@@]1(N=[N+]=[N-])C(=O)O]

(3R,4S)-4-allyl-3-azido-1-(tert-butoxycarbonyl)pyrrolidine-3-carboxylic acid or salt thereof.

In some embodiments, the present disclosure provides a compound of formula 11:

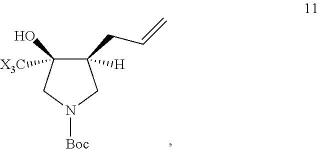

or a salt thereof, wherein:
each X is independently selected from hydrogen or halogen (including chloro, bromo, or iodo);
PG³ is a suitable amine protecting group.

In certain embodiments, the compound of formula 11 is:

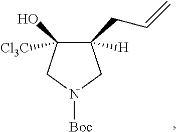

tert-butyl (3S,4S)-4-allyl-3-hydroxy-3-(trichloromethyl)pyrrolidine-1-carboxylate or salt thereof.

In certain embodiments, the compound of formula 11 is:

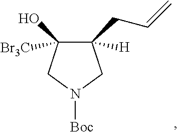

tert-butyl (3S,4S)-4-allyl-3-hydroxy-3-(tribromomethyl)pyrrolidine-1-carboxylate or salt thereof.

In some embodiments, the present disclosure provides a compound of formula 20c

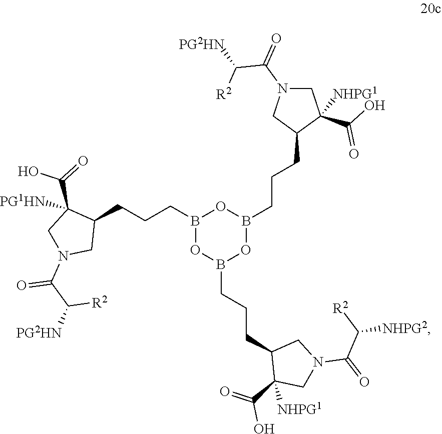

or a salt thereof, wherein:
R² is H, (C₁-C₆)alkyl, or —CH₂OH; and
PG¹ and PG² are independently suitable amine protecting groups.

In some embodiments of the compounds of Formulas 20, 19, 18, 17, 15, 14 or 13, suitable amine protecting groups for PG¹, PG² and PG³ are independently selected from tert-butyloxycarbonyl, ethyloxycarbonyl, methyloxycarbonyl, trichloroethyloxycarbonyl, allyloxycarbonyl, benzyloxocarbonyl, allyl, benzyl, fluorenylmethylcarbonyl, acetyl, chloroacetyl, dichloroacetyl, trichloroacetyl, trifluoroacetyl, phenylacetyl, and benzoyl.

In some embodiments of the compounds of Formulas 20c, 20, 19, or 18, R² is H, methyl, or —CH₂OH.

In some embodiments of the compounds of Formulas 20, 19, 18, 17, 15, 14 or 13, R³ is

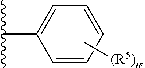

wherein each R⁵ is independently selected from halo, nitro, azido, cyano, aldehyde, amide, carboxylic acid, amino, hydroxyl, thiol, —(C₁-C₆)alkyl, —NH(C₁-C₆)alkyl, —N[(C₁-C₆)alkyl]₂, —CO(C₁-C₆)alkyl, —CO₂(C₁-C₆)alkyl, —O(C₁-C₆)alkyl, —S(C₁-C₆)alkyl, —NHCO(C₁-C₆)alkyl, and —NHCO₂(C₁-C₆)alkyl, aryl, and heteroaryl, and n is an integer from 0-5. In certain embodiments, R³ is phenyl.

In some embodiments of the compounds of Formulas 20, 19, 18, 17, 15, 14 or 13, R³ is a heteroaryl ring substituted with (R⁵)ₙ, wherein each R⁵ is independently selected from halo, nitro, azido, cyano, aldehyde, amide, carboxylic acid, amino, hydroxyl, thiol, —(C₁-C₆)alkyl, —NH(C₁-C₆)alkyl, —N[(C₁-C₆)alkyl]₂, —CO(C₁-C₆)alkyl, —CO₂(C₁-C₆)alkyl, —O(C₁-C₆)alkyl, —S(C₁-C₆)alkyl, —NHCO(C₁-C₆)alkyl, and —NHCO₂(C₁-C₆)alkyl, aryl, and heteroaryl, and n is an integer from 0-5.

In some embodiments of the compounds of Formulas 20c, 20, 19, 18, 17, or 15, PG¹ is benzyloxocarbonyl. In some embodiments of the compounds of Formulas 20c, 20, 19, or 18, PG² is benzyloxocarbonyl. In some embodiments of the compounds of Formulas 20c, 15, 14, 13, 12, or 11, PG³ is tert-butyloxycarbonyl.

### 3.7. Pharmaceutical Compositions

In some embodiments, provided herein is a process for preparing a pharmaceutical composition comprising mixing (i) a compound of formula 21 or formula 22, or a pharmaceutically acceptable salt thereof prepared according to any of the processes described herein, and (ii) a pharmaceutically acceptable carrier.

Pharmaceutical compositions containing a compound of formula 21 or formula 22, or a pharmaceutically acceptable salt thereof as the active ingredient can be prepared by intimately mixing a compound of formula 21 or formula 22, or a pharmaceutically acceptable salt thereof with a pharmaceutical carrier according to conventional pharmaceutical compounding techniques. The carrier can take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents, and the like; for solid oral preparations, such as powders, capsules and tablets, suitable carriers and additives include starches, sugars, diluents, granulating agents, lubricants, binders, disintegrating agents, and the like. Solid oral preparations can also be coated with substances such as sugars or be enteric-coated so as to modulate major site of absorption. For parenteral administration, the carrier will usually consist of sterile water, and other ingredients can be added to increase solubility or preservation. Injectable suspensions or solutions can also be prepared utilizing aqueous carriers along with appropriate additives.

A compound of formula 21 or formula 22, or a pharmaceutically acceptable salt thereof can be administered by any convenient route, e.g., into the gastrointestinal tract (e.g., rectally or orally), the nose, lungs, musculature or vasculature, or transdermally or dermally. A compound of formula 21 or formula 22, or a pharmaceutically acceptable salt thereof can be administered in any convenient administrative form, e.g., tablets, powders, capsules, solutions, dispersions, suspensions, syrups, sprays, suppositories, gels, emulsions, patches, etc. Such compositions can contain components that are conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion. Such compositions form a further aspect of the present disclosure.

Also provided herein are pharmaceutical compositions comprising a compound of formula 21 or formula 22, or a pharmaceutically acceptable salt thereof. To prepare the pharmaceutical compositions provided herein, a compound of formula 21 or formula 22, or a pharmaceutically acceptable salt thereof as the active ingredient is intimately admixed with a pharmaceutical carrier according to conventional pharmaceutical compounding techniques. The carrier can take a wide variety of forms depending of the form of preparation desired for administration, e.g., oral or parenteral, such as intramuscular. Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain. Methods of formulating pharmaceutical compositions have been described in numerous publications, such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

### 4. EXAMPLES

This section will describe the various different working examples that will be used to highlight the features of the present disclosure.

The starting materials and reagents used in the preparation of the compounds in the present disclosure are either available from commercial suppliers such as Sigma-Aldrich (St. Louis, Mo.) or Fisher Scientific (Hampton, N.H.) or are prepared by methods known to those skilled in the art following procedures set forth in references such as Fieser and Fieser's Reagents for Organic Synthesis, Volumes 1-17 (John Wiley and Sons, 1991), Organic Reactions, Volumes 1-40 (John Wiley and Sons, 1991), and March's Advanced Organic Chemistry (John Wiley and Sons, 4^{th} Edition).

### Example 1: Synthesis of (3R,4S)-3-benzyl 1-tert-butyl 4-allyl-3-(benzyloxycarbonylamino)pyrrolidine-1,3-dicarboxylate from L(−)-malic Acid

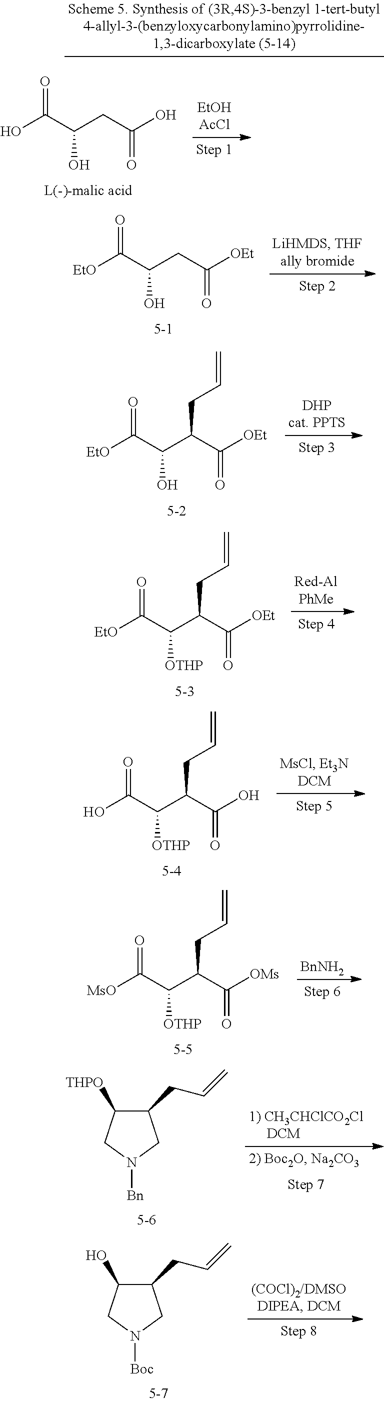

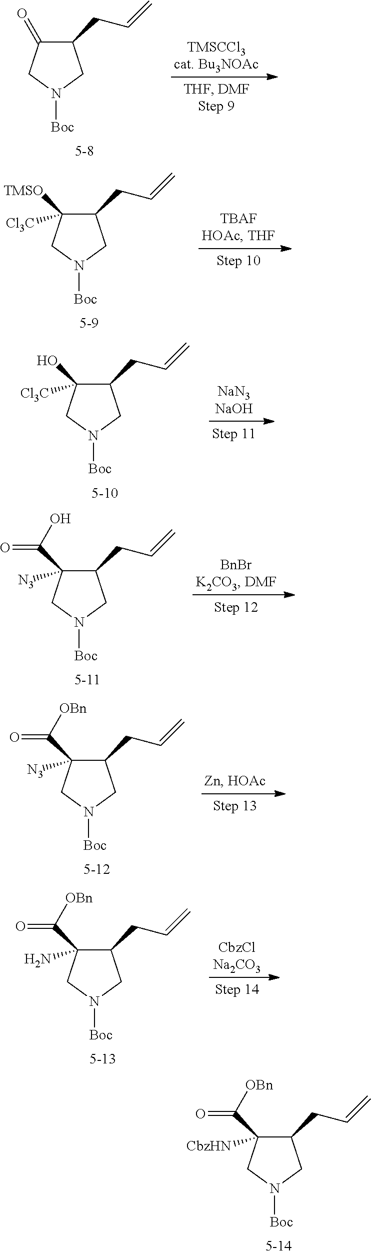

All yields for the synthesis of 5-12 are considered crude and uncorrected.

Step 1: (S)-Diethyl 2-hydroxysuccinate (5-1). To an inerted reactor was charged ethanol (2400 kg). Agitation was started and upon cooling to 0±10° C., acetyl chloride (708 kg, 9,000 mol) was added slowly over the course of approx. 5 hrs. Next, L(−)-malic acid (600 kg, 4,470 mol) was charged to the mixture while maintaining the batch between −10 to −20° C. The mixture was then warmed to 23-27° C. and stirred for 18 hrs after which the starting material was deemed consumed by TLC. The batch was then concentrated under reduced pressure and the residue was diluted with DCM (1050 kg) and water (900 kg). After agitating for 15 min., agitation was stopped and the phases were separated. The aqueous phase was back-extracted with DCM (600 kg). The organic phases were combined and washed with 8% aqueous sodium bicarbonate (1100 kg) and 20% brine (600 kg). After drying over Na₂SO₄ (174 kg), the batch was filtered and concentrated under reduced pressure. THF (250 kg) was charged to the residue and the solution was evaporated to dryness to afford 5-1 as yellow oil (569 kg, 66.9% yield, 93% purity) which was used without further purification.

Step 2: (2R,3S)-Diethyl 2-allyl-3-hydroxysuccinate (5-2). To an inerted reactor was charged THF (855 kg) and HMDS (320 kg, 1.98 mol) at ambient temperature. Agitation was started and the mixture was cooled to about −70° C. At this temperature, 2.5M n-BuLi (500 kg, 1843 Mol) was added slowly while the temperature was maintained at −60±5° C. The mixture was stirred for 0.5 h at this temperature. Next, a solution of 5-1 (171 kg) from the previous step in THF (171 kg) was added slowly while the temperature was maintained at −60±5° C. The mixture was stirred for 1.0 h at this temperature. Next allyl bromide (163.3 kg, 1349 mol) was added slowly while the temperature was being maintained at −60±5° C. The mixture was stirred for 1.0 h after addition and warmed to 10±5° C. After stirring for 2 hrs at this temperature, the reaction was deemed complete by HPLC. The mixture was then quenched by slow addition of aqueous 20% citric acid (1400 kg) keeping the temperature below 15° C. The mixture was agitated for 15 min and upon ceasing agitation, the phases were separated. The aqueous phase was back-extracted with MTBE (513 kg) and the combined organic phases were washed with 10% brine (496 kg). After being dried with Na₂SO₄ (50 kg), the solution was concentrated to dryness to afford crude (2R,3S)-diethyl 2-allyl-3-hydroxysuccinate (5-2) as a yellow oil (192.0 kg, 92.7% yield, ˜9:1 mixture of diastereoisomers, 75.6% purity), which was used without further purification in the next step.

Step 3: (2R,3S)-Diethyl 2-allyl-3-(tetrahydro-2H-pyran-2-yloxy)succinate (5-3). To a reactor was charged dichloromethane (2100 kg), crude (2R,3S)-diethyl 2-allyl-3-hydroxysuccinate (5-2) (700 kg, 75.6% purity), dihydropyran (384 kg, 4561 mol) and PPTS (21 kg, 83.6 mol). Agitation was started and the mixture was heated to 35±3° C. and stirred for 8 hrs after which it was deemed complete by HPLC. The mixture was then cooled to 20±10° C. Next water (1400 kg) was added and the organic layer was separated. The aqueous phase was then back-extracted with DCM (1400 kg) and the combined organic phases were washed with 10% brine (700 kg), dried over Na₂SO₄ (50 kg) and filtered. The filtrate was evaporated to dryness under reduced pressure to afford crude 5-3 (1032.1 kg, 99.3% yield, 75.3% purity) as a yellow oil which was used in the next step without further purification.

Step 4: (2S,3S)-2-Allyl-3-(tetrahydro-2H-pyran-2-yloxy)butane-1,4-diol (5-4). To an inerted reactor was charged toluene (720 kg) and 70% Red-Al® in toluene (826 kg) under nitrogen. Agitation was started and the mixture was cooled to 0-10° C. A solution of (2R,3S)-diethyl 2-allyl-3-(tetrahydro-2H-pyran-2-yloxy)succinate (5-3) (360 kg, 75.3% purity) in toluene (360 kg) was added slowly while maintaining the temperature between 0-10° C. After the addition, the mixture was warmed to 20-30° C. and stirred for 4 hrs and the reaction was deemed complete by HPLC. The reaction mixture was then added to a solution of potassium sodium tartrate (1616 kg) in water (2412 kg) and the organic layer was separated after agitation. The aqueous phase was back-extracted with MTBE twice (2×720 kg). The combined organic phases were washed with 25% brine (504 kg). The aqueous phase was back-extracted with MTBE (360 kg). The organic phases were combined, dried over Na₂SO₄ (50 kg) and filtered. The filtrate was concentrated to afford 5-4 (227.4 kg, 87.4% yield, 88.7% purity) as a yellow oil which was used directly for the next step.

Step 5: (2S,3S)-2-Allyl-3-(tetrahydro-2H-pyran-2-yloxy)butane-1,4-diyl dimethanesulfonate (5-5). To a reactor was charged crude (2S,3S)-2-allyl-3-(tetrahydro-2H-pyran-2-yloxy)butane-1,4-diol (5-4). (332 kg, 88.7% purity), Et3N (486 kg, 4812 mol) and dichloromethane (1476 kg). Agitation was started and the mixture was cooled to 0±10° C. At this temperature, methanesulfonyl chloride (441 kg, 3851 mol) was added slowly over the course of approx. 12 hrs. The mixture was warmed to 15-25° C. and stirred for 1 h at this temperature. No starting material was detected by TLC. The mixture was quenched by slow addition to water (996 kg), keeping the internal temperature below 15° C. Agitation of the mixture was continued for 15 min and the phases were separated. The aqueous phase was back-extracted with DCM (738 kg). The combined organic phase was washed with 20% brine (664 kg). After being dried with Na₂SO₄ (45 kg), the batch was concentrated under reduced pressure to afford crude 5-5 (527.7 kg, 94% yield) as a brown oil which was used directly for the next step without further purification.

Step 6: (3S,4S)-3-Allyl-1-benzyl-4-(tetrahydro-2H-pyran-2-yloxy)pyrrolidine (5-6). To a reactor was charged diisopropylethylamine (530.1 kg, 4108 mol), crude (2S,3S)-2-allyl-3-(tetrahydro-2H-pyran-2-yloxy)butane-1,4-diyl dimethanesulfonate (5-5) (527.7 kg) from the previous step and benzylamine (292.3 kg, 2728 mol). The mixture was agitated and heated to 55-65° C. for 2 hrs. Next, the mixture was heated to 97-99° C. and stirred for 7 hrs where TLC showed no starting material. The mixture was allowed to cool to 10-30° C. Next, CH₃CN (528 kg) and Et₃N (276 kg, 2727 mol) were added to the reaction mixture, followed by addition of succinic anhydride (178 kg, 1775 mol) over the course of 2 hrs while the internal temperature was being maintained at 15-30° C. The mixture was stirred for 2 hrs at this temperature until benzylamine was consumed. The batch was next concentrated under reduced pressure. A solution of Na₂CO₃ (1.0 eq) in water (1056 kg) was then added slowly while maintaining the temperature between 10-25° C. The mixture was next diluted with MTBE (1056 kg). Agitation of the mixture was continued for 15 min and the phases were separated. The aqueous phase was back-extracted with MTBE (528 kg). The combined organic phases were washed with a solution of Na₂CO₃ (144 kg) in water (972 kg) and 10% brine (1056 kg). After being dried with Na₂SO₄ (50 kg), the batch was filtered. The filtrate was concentrated under reduced pressure to afford crude 5-6 (369.5 kg, 89.8% yield, 90.7% purity) as a brown oil which was used without further purification in the next step.

Step 7: (3S,4S)-tert-Butyl 3-allyl-4-hydroxypyrrolidine-1-carboxylate (5-7). To a reactor was charged dichloromethane (1536 kg) and 1-chloroethyl chloroformate (273 kg, 1906 mol) under nitrogen and at ambient temperature. Agitation was started and the mixture was cooled to between −8 and −11° C. Diisopropylethylamine (49.4 kg, 383 mol) was added slowly while the temperature was being maintained between −8 and −11° C. The mixture was stirred for 5 mins at this temperature. Next a solution of crude 5-6 (384 kg) in DCM (384 kg) was added slowly while the temperature was being maintained between −8 and −11° C. After the addition, the mixture was warmed to NMT 20° C. and stirred for 1 h at this temperature. Upon consumption of starting material, the reaction mixture was added to MeOH (1536 kg) while the temperature was being maintained between 5 and 25° C. The batch was stirred for 8 hrs at this temperature and then concentrated under reduced pressure. The residue was diluted with MTBE (768 kg) and water (1920 kg). The mixture was agitated for 15 min and the phases were separated. The aqueous phase was back-extracted with MTBE (768 kg) and then a solution of Na₂CO₃ (406 kg) in water (1574 kg) was added slowly while the temperature was being maintained between 10 and 25° C. A solution of Boc anhydride (277 kg, 1271 mol) in THF (384 kg) was added slowly while maintaining the temperature between 0 and 10° C. The mixture was warmed to ambient temperature and stirred for 8 hrs. The mixture was next diluted with MTBE (768 kg) and water (3000 kg). Agitation of the mixture continued for 15 min and ceased wherein the phases were separated. The aqueous phase was then back-extracted with MTBE (768 kg). The combined organic phases were washed with 10% brine (768 kg). After being dried with Na₂SO₄ (50 kg), the batch was concentrated under reduced pressure to give a brown solid. The brown solid was suspended in petroleum ether (768 kg) and heated to 50-60° C. until a solution was obtained. The solution was allowed to cool slowly to 40° C. under agitation and aged for 2 hrs at this temperature. The slurry was further cooled to 10-15° C. and the solids were collected by filtration. The filter cake was rinsed with cold petroleum ether (384 kg) to give 5-7 (158.5 kg, 55.1% yield, 99.3% purity) as a yellow solid.

Step 8: (S)-tert-butyl 3-allyl-4-oxopyrrolidine-1-carboxylate (5-8). To a reactor was charged DCM (670 kg) and oxalyl chloride (7.3 kg, 57.5 mol) under nitrogen. Agitation was started and the mixture was cooled to between −60 and −65° C. At this temperature, a solution DMSO (5.2 kg. 66.6 mol) in dichloromethane (13 kg) was added slowly while maintaining the temperature between at −54 and −65° C. The mixture was stirred for 0.5 h at this temperature. A solution of 5-7 (10 kg) in dichloromethane (27 kg) was added slowly while maintaining the temperature being between −54 and −65° C. The mixture was stirred for 1.0 h at this temperature. Diisopropylethyl amine was added slowly while maintaining the temperature being between −54 and −65° C. Next, the mixture was stirred for 1.0 h at this temperature and warmed to about −30° C. The reaction was stirred for 2.0 h at this temperature and deemed complete by HPLC. The reaction solution was next charged to a mixture citric acid monohydrate (4.6 kg) and water (50 kg). The organic layer was separated and the aqueous layer extracted with DCM (66 kg). The DCM extracts were combined and washed successively with a solution of citric acid monohydrate (2.7 kg) in H₂O (50 kg), H₂O (30 kg×4), 10% brine (20 kg). After drying with Na₂SO₄ (10 kg), the solution was concentrated to 2.5V. Next, THF (18 kg) was added to the reactor followed by concentrating the solution to 2V. THF (18 kg) was added again to the reactor and the solution was concentrated to 2V to afford 5-8 (80.6% purity, 97.8% ee) in THF which was used directly for the next reaction.

Step 9: (3S,4S)-tert-Butyl 4-allyl-3-(trichloromethyl)-3-(trimethylsilyloxy)pyrrolidine-1-carboxylate (5-9). To a reactor was charged THF (14.4 kg), DMF (14.4 kg) and TMSCCl₃ (12.8 kg, 66.8 mol) under nitrogen. Agitation was started and the mixture was cooled to about −20° C. At this temperature, the solution of 5-8 in THF from the previous step was added slowly while maintaining the temperature between −25 and −20° C. After the addition, a solution of tetrabutylammonium acetate (1.4 kg, 4.6 mol) in DMF (14.4 kg) was added slowly while the temperature being maintained between −25 and −20° C. The reaction mixture was stirred for 1 h at this temperature or until starting material was consumed. The reaction solution was charged to a mixture of sat. NH₄Cl (128 kg) and MTBE (74 kg) with agitation. After agitation stopped, the organic layer was separated and the aqueous layer back-extracted with MTBE (37 kg). The organic extracts were combined and washed twice with H₂O (60 kg total). After being dried with Na₂SO₄ (15 kg), the solution was concentrated to dryness to afford 5-9 (60.3% purity) as a brown oil which was used directly in the next step.

Step 10: (3S,4S)-tert-Butyl 4-allyl-3-hydroxy-3-(trichloromethyl)pyrrolidine-1-carboxylate (5-10). To an inerted reactor was charged THF (26.7 kg) and 5-9 from the previous reaction. The mixture was agitated and cooled to between 0 and 10° C. At this temperature, a solution of TBAF (14.4 kg, 453 mol) and AcOH (2.7 kg, 453 mol) in THF (18 kg) was added slowly while the temperature was kept between 0 and 10° C. The reaction mixture was stirred at this temperature for 1 h and deemed complete by HPLC. The mixture was then added to an aqueous solution of NaHCO₃ (4.2 kg, 500 mol) in H₂O (50 kg) and ethyl acetate (45.1 kg). The organic layer was separated and the aqueous layer extracted twice with EA (50 kg total). The organic layers were combined and washed twice with half sat. brine (47 kg) and sat. brine (27 kg). After being dried with Na₂SO₄ (38 kg), the solution was concentrated to dryness. The residue was dissolved in PE (54 kg) and EA (5.7 kg). The mixture was warmed until most of solid dissolved. The solution was cooled slowly to 0° C. and filtered. The cake was dried under reduced pressure to afford 5-10 (11.2 kg, 74% based on 5-7, 98.8% purity, 100% ee), as an off-white solid.

Step 11: (3R,4S)-4-allyl-3-azido-1-(tert-butoxycarbonyl)pyrrolidine-3-carboxylic acid (5-11). To an inerted reactor was charged 1,4-dioxane (44.5 kg) and (3S,4S)-tert-Butyl 4-allyl-3-hydroxy-3-(trichloromethyl)pyrrolidine-1-carboxylate 5-10 (11 kg) from the previous reaction. Agitation was started and the mixture was cooled to between 8-13° C. At this temperature, a solution NaN₃ (3.1 kg, 47.7 mol), NaOH (3.8 kg, 95 mol) in H₂O (44 kg) was added slowly while the temperature was being maintained between 10-15° C. Agitation was continued for 1 h. The reaction solution was then warmed to about 20° C. and stirred for 1 h. The reaction solution was then warmed to 25-32° C. and stirred for 2 hrs and deemed complete by HPLC. The mixture was next added to sat. aq. NH₄Cl (33.3 kg) and ethyl acetate (49.5 kg). The organic layer was separated and the aqueous layer back-extracted twice with ethyl acetate (99 kg total). The organic layers were combined and washed twice with half sat. brine (52 kg total). The aqueous layer was next back-extracted with EA (30 kg). The organic layers were combined and washed with aqueous citric acid monohydrate (7.3 kg) in H₂O (36.3 kg). The organic layer was separated and washed with sat. brine (30 kg). After being dried with Na₂SO₄ (11 kg), the solution was concentrated to dryness to afford 5-11 as a yellow oil and used directly in the next step.

Step 12: (3R,4S)-3-benzyl 1-tert-butyl 4-allyl-3-azidopyrrolidine-1,3-dicarboxylate (5-12). To a reactor were charged DMF (26.9 kg) and (3R,4S)-4-allyl-3-azido-1-(tert-butoxycarbonyl)pyrrolidine-3-carboxylic acid (5-11) from the previous reaction. Next, K₂CO₃ (3.6 kg, 28.6 mol) was added to the reaction solution while the temperature being kept at 15-30° C., followed by addition of BnBr (4.9 kg, 28.6 mol). After the addition, the reaction mixture was heated to 35-45° C. Agitation was continued at 35-45° C. for 4 hrs where the reaction was deemed complete by HPLC. The reaction mixture was then charged to a mixture of MTBE (35.8 kg) and H₂O (47 kg) while maintaining the temperature between 0 and 10° C. The organic layer was separated and the aqueous layer extracted with MTBE twice (36 kg total). The MTBE extracts were combined and washed with H₂O (38 kg). After being dried with Na₂SO₄ (5.6 kg), the solution was concentrated to dryness to afford 5-12 (9.4 kg, 76.4% yield based on 5-10, 89.4% purity) as a yellow oil which was used directly in the next reaction.

Step 13: (3R,4S)-3-benzyl 1-tert-butyl 4-allyl-3-aminopyrrolidine-1,3-dicarboxylate (5-13). To an inerted reactor was charged THF (34 kg), AcOH (39.5 kg) and crude (3R,4S)-3-benzyl 1-tert-butyl 4-allyl-3-azidopyrrolidine-1,3-dicarboxylate (5-12) from the previous reaction. Agitation was started and the mixture was heated to between 38-42° C. Zinc powder (3.4 kg, 52 mol) was added to the mixture while the reaction temperature was being maintained between 59±3° C. At this temperature, the reaction mixture was stirred for 1 h after which it was deemed complete. The mixture was filtered and the filter cake washed with THF (3.8 kg). The filtrate was evaporated and to the residue was charged MTBE (35.7 kg) and H₂O (37.6 kg). The pH of the reaction mixture was brought up to 8 by addition of NH₄OH. The organic layer was separated and the aqueous layer extracted with MTBE twice (42 kg total). The organic layers were combined and washed twice with H₂O (18.9 kg total). After being dried with Na₂SO₄ (7.5 kg), the solution was concentrated to dryness. To the resulting residue was charged acetonitrile (58.8 kg) and the batch was heated to between 55-68° C. A solution of L(+)-tartaric acid (3.65 kg, 24.3 mol) in THF (44 kg) was added to the mixture while the temperature was between 55-65° C. After the addition, the batch was cooled to 0° C. and stirred for 0.5 h. The suspension was filtered and the solids were washed with MeCN (15 kg). The wet cake was charged to a mixture of MTBE (28.2 kg) and H₂O (28.2 kg). The pH of the mixture was brought to 8 by addition of NaHCO₃ (4.2 kg, 50 mol). The organic layer was separated and the aqueous layer extracted twice with MTBE (35 kg total). The organic extracts were combined and washed twice with H₂O (18.8 kg total). After being dried with Na₂SO₄ (7.5 kg), the filtered solution was concentrated to dryness to afford 5-13 as a colorless to light yellow oil which was used directly in the next reaction.

Step 14: (3R,4S)-3-benzyl 1-tert-butyl 4-allyl-3-(benzyloxycarbonylamino)pyrrolidine-1,3-dicarboxylate (5-14). To an inerted reactor was charged THF (31.5 kg), 5-13 from the previous reaction, H₂O (35 kg), NaHCO₃ (0.5 kg) and Cbz-OSu (4.85 kg). Agitation was started and the mixture was heated to 35-40° C. Agitation was continued at 35-40° C. for 8 hrs where the reaction was deemed complete. The reaction mixture was cooled to 20±10° C. NaHCO₃ (1.6 kg) was added slowly to the mixture, followed by EA (28 kg). The organic layer was separated and the aqueous layer extracted with ethyl acetate (31.5 kg). The organic layers were combined and washed with 5% aqueous NaHCO₃ (14.7 kg), half sat. brine (25 kg) and sat. brine (19 kg). After being dried with Na₂SO₄ (7. Kg), the solution was concentrated to dryness. Next MeCN (16.8 kg) was charged. The mixture was heated to 60-81° C. and H₂O (28 kg) was added. The solution was cooled slowly to about 0° C. and filtered. The wet cake was washed with a mixture of MeCN (4.97 kg) and H₂O (8.4 kg). The wet cake was suspended in MeCN (17 kg) and the mixture was heated to 70±10° C. and H₂O (28 kg) was added. The solution was cooled slowly to about 0° C. and filtered. The filter cake was washed with a mixture of MeCN (5 kg) and H₂O (8.4 kg). The cake was dried under reduced pressure at about 55° C. to afford 5-14 as white solid (8.96 kg, 56.8% yield based on 5-10 initial charge of 11 kg, 99% purity, greater than 99.95% ee. ¹H NMR (500 MHz, DMSO) δ 8.26 (d, J=10.1 Hz, 1H), 7.46-7.30 (m, 10H), 5.74-5.52 (m, 1H), 5.23-5.08 (m, 2H), 5.08-4.95 (m, 4H), 4.18 (d, J=11.4 Hz, 1H), 3.47 (dd, J=10.7, 7.5 Hz, 1H), 3.38-3.24 (m, 1H), 2.95 (dt, J=10.9, 7.7 Hz, 1H), 2.48-2.36 (m, 1H), 2.36-2.23 (m, 1H), 1.58 (dt, J=12.0, 6.3 Hz, 1H), 1.38 (s, 9H); HRMS (ESI⁺): [M+Na]⁺ calc. m/z=517.2309, found m/z=517.2315.

### Example 2: Alternate Synthesis of (3R,4S)-3-benzyl 1-tert-butyl 4-allyl-3-(benzyloxycarbonylamino)pyrrolidine-1,3-dicarboxylate Starting from diethyl (2S,3S)-2,3-dihydroxysuccinate

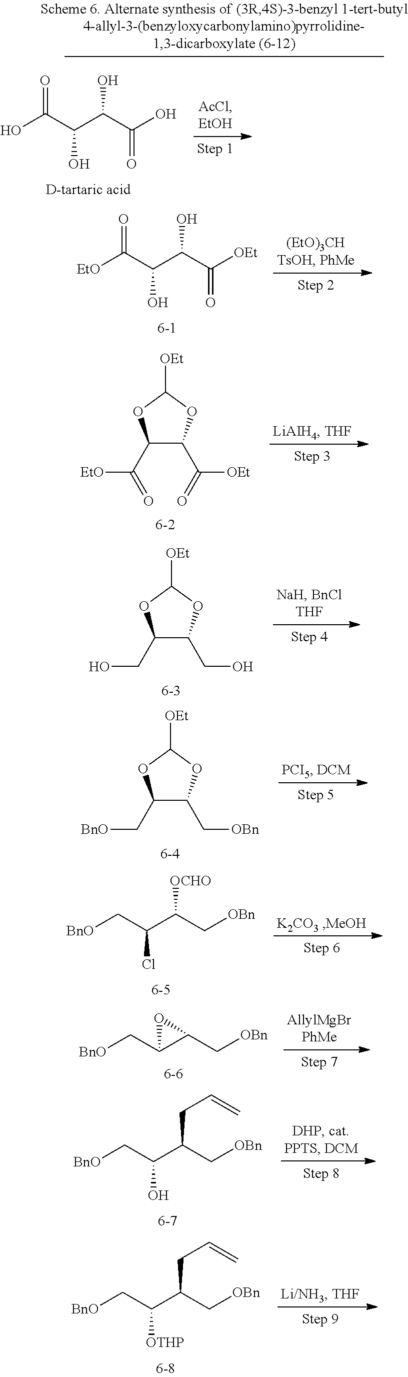

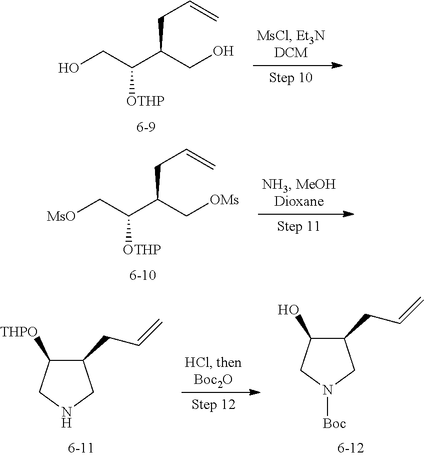

Step 1: Diethyl D-tartrate (6-1). To a three-necked flask was added anhydrous EtOH (5V) under nitrogen. Next, acetyl chloride (2.0 eq) was added dropwise at −10 to 10° C., followed by D-tartaric acid (1.0 eq). The reaction temperature was raised to 23-27° C. and maintained at that temperature for 18 hours after which the reaction was deemed complete. The mixture was then concentrated to dryness. Next, dichloromethane (5V) was added with agitation, followed by water (5V). After stopping agitation, the phases were separated. The organic phase was washed with a sodium bicarbonate solution and a 20% sodium chloride solution and the solvent was removed to afford the titled 6-1 as a yellow oil (75.8% yield) used without further purification in the next step.

Step 2: Diethyl (4S,5S)-2-ethoxy-1,3-dioxolane-4,5-dicarboxylate (6-2). To a flask was added 6-1 (1.0 eq), triethyl orthoformate (2.7 eq), p-toluenesulfonic acid (0.01 eq) and toluene (4V). The reaction mixture was heated to 100-110° C. Ethanol was removed by distillation with a Dean-Stark trap for 8 hours and toluene was replenished until completion by TLC. The reaction mixture was cooled to room temperature and washed with a NaHCO₃ solution (2V). The organic phase was concentrated to dryness to afford the titled 6-2 (99.8% yield, GC 95.9% purity) and used without further purification in the next step.

Step 3: ((4R,5R)-2-Ethoxy-1,3-dioxolane-4,5-diyl)dimethanol (6-3). To a flask was added LiAlH₄ (1.0 eq) and THF (6V) under nitrogen. The mixture was cooled to 0° C. with an ice-salt bath. 6-2 (1.0 eq) was added dropwise and the reaction temperature was maintained at 0-10° C. After the addition was completed, the reaction mixture was stirred at 25° C. for 1 hour and shown to be complete by TLC. The reaction mixture was cooled to 0° C. Na₂SO₄.10H₂O was next added in portions and the temperature was maintained 0-10° C. After the addition, the reaction mixture was stirred at room temperature for 1 hour and filtered. The solvent in the filtrate was removed to afford the titled 6-3 as an oil (73% yield, 90.1% GC purity).

Step 4: (4R,5R)-4,5-Bis((benzyloxy)methyl)-2-ethoxy-1,3-dioxolane (6-4). THF (4V), DMF (1V), and NaH (2.5 eq) were added to a flask under nitrogen. The mixture was then cooled to 0° C. Next a solution of 6-3 (1.0 eq) in THF (1V) was added dropwise. After the addition, the reaction mixture was allowed to stir at room temperature for 1 hour. The reaction mixture was then cooled to 0° C. and benzyl chloride (2.5 eq) was added drop wise at 0-10° C. After the addition, the reaction mixture was stirred at room temperature overnight. Upon completion, the reaction was quenched by adding the mixture to a NaHCO₃ solution. The mixture was then extracted with methyl t-butyl ether twice. The combined organic phase was washed with water, followed by saturated brine. The organic phase was then dried with anhydrous Na₂SO₄ and filtered. The solvent was removed to afford the titled 6-4 as an oil (107% yield containing unreacted BnCl).

Step 5: (2R,3S)-1,4-Bis(benzyloxy)-3-chlorobutan-2-yl formate (6-5). To a flask was added CH₂Cl₂ (4V) and PCl₅ (1.2 eq) under nitrogen. Next, a solution of 6-4 (1.0 eq) in DCM (1V) was added dropwise and the temperature was maintained at 0-10° C. The mixture was cooled to 0° C. After the addition, the reaction mixture was stirred at room temperature overnight. Upon completion, the reaction was quenched by adding the mixture to a NaHCO₃ solution. After phase separation, the organic phase was washed with a NaHCO₃ solution and a saturated NaCl solution. The organic phase was dried with anhydrous Na₂SO₄ and filtered. The filtrate was concentrated to afford the titled 6-5 as an oil (92.4% yield) which was used without further purification in the next step.

Step 6: (2R,3R)-2,3-Bis((benzyloxy)methyl)oxirane (6-6). Methanol (5V), 6-5 (1.0 eq), and K₂CO₃ (3.0 eq) were added to a flask. The mixture was stirred at room temperature overnight. Upon completion, the reaction mixture was concentrated to remove most of the methanol. Water (5V) was added and the mixture was extracted with MTBE (2V x 2). The combined organic phase was washed once with water and once with saturated brine. The organic phase was dried with anhydrous Na₂SO₄ and then filtered. After removal of the solvent, the residue was dissolved in petroleum ether (2V) and cooled to −50° C. A precipitate was observed, collected by filtration and dried to afford the titled 6-6 as a light-yellow solid (61.2% yield, 95.2% GC purity, 99.9% HPLC purity)

Step 7: (2S,3S)-1-(Benzyloxy)-3-((benzyloxy)methyl)hex-5-en-2-ol (6-7). To a flask was added a solution of allylmagnesium bromide (2.5 eq) in THF. The solvent was removed by distillation. Toluene (5V) was then added and the solvent was again removed by distillation. Toluene (5V) was added under nitrogen and the reaction mixture was cooled to 0° C. A solution of 6-6 (1.0 eq) in toluene (1V) was added dropwise and the reaction temperature was maintained at −5 to 5° C. After the addition, the reaction mixture was stirred at that temperature for an additional 30 min was judged complete by HPLC. The reaction mixture was then added to a 20% NH₄Cl solution. After phase separation, the aqueous phase was extracted with MTBE (10V). The combined organic phase was washed with water, dried with anhydrous Na₂SO₄ and filtered. The filtrate was concentrated to dryness to give the titled 6-7 as an oil (99% yield, 97.9% HPLC purity) which was used without further purification in the next step.

Step 8: 2-(((2S,3S)-1-(Benzyloxy)-3-((benzyloxy)methyl)hex-5-en-2-yl)oxy)tetrahydro-2H-pyran (6-8). DCM (3V), 3,4-dihydropyran (1.5 eq), 6-7 (1 eq) and PPTS (0.02 eq) were added to a flask and the mixture stirred at room temperature overnight. Upon completion, the reaction mixture was washed with a NaHCO₃ solution. The aqueous layer was back extracted with DCM (3V) and the combined organic phase was washed with water (2V), dried with anhydrous Na₂SO₄ and filtered. The solvent was removed from the filtrate to afford the titled 6-8 as an oil (99.5% yield) which was used without further purification in the next step

Step 9: (2S,3S)-2-Allyl-3-((tetrahydro-2H-pyran-2-yl)oxy)butane-1,4-diol (6-9). To a flask was added THF (2V) and 6-8 (1.0 eq) under nitrogen. The mixture was cooled to −60° C. and ammonia (6V) was bubbled through the reaction mixture. Lithium wire (4.1 eq) was cut and added in portions while maintaining the reaction temperature at −60 to −50° C. Upon completion the reaction mixture was slowly warmed to room temperature to evaporate the ammonia. DCM (10V) and anhydrous Na₂SO₄ (2.0 wt) were added to the reaction mixture. After stirring, the mixture was filtered. The solids were rinsed with DCM and the filtrate was concentrated to dryness to afford the titled 6-9 as an oil (100% yield).

Step 10: (2S,3S)-2-Allyl-3-((tetrahydro-2H-pyran-2-yl)oxy)butane-1,4-diyl dimethanesulfonate (6-10). DCM (4V), 6-9 (1.0 eq), and Et₃N (3.0 eq) were added to a flask under nitrogen. The mixture was cooled to 0° C. Next MsCl (2.4 eq) was added dropwise while the reaction temperature was maintained at 0-10° C. The reaction mixture was then stirred at room temperature for 1 hour after the addition. After completion, the reaction mixture was added to ice water (5V). The phases were separated and the water phase was back extracted with DCM (4V). The combined organic phase was washed with water (5V). The organic phase was dried with anhydrous Na₂SO₄ and filtered. The solvent was removed from the filtrate to afford the titled 6-10 as an oil (100% yield) which was used without further purification in the next step.

Step 11: (3S,4S)-3-Allyl-4-((tetrahydro-2H-pyran-2-yl)oxy)pyrrolidine (6-11). To a chilled pressure vessel was added a 50% methanol solution (20V) and a solution of 6-10 (1.0 eq) in 1,4-dioxane (1V). The pressure vessel was closed and heated to 60° C. and kept at that temperature overnight. TLC showed incomplete reaction, LC-MS found the presence of a dimer as side product. After cooling, the solvent was evaporated and DCM (10V), added to the residue followed by a solution of Na₂SO₄. pH was adjusted to 13 with NaOH. The phases were then separated and the aqueous phase was back extracted with DCM. The combined organic phase was concentrated to afford the titled 6-11 as an oil (110% yield containing salts).

Step 12: tert-Butyl (3S,4S)-3-allyl-4-hydroxypyrrolidine-1-carboxylate (6-12). THF (5V), 6-11 (1.0 eq) from the previous reaction, water (10V), and hydrochloric acid (2.2 eq) were added to a flask and the mixture stirred at room temperature for 2 hours. TLC showed that the cyclization was complete. Next THF was removed and the reaction mixture was extracted with MTBE (5V×2). The organic phase was discarded. Sodium carbonate (3.0 eq) was then added to the aqueous phase. Next, a solution of (Boc)₂O (1.5 eq) in THF (5V) was added dropwise. The mixture was stirred at room temperature overnight. Upon completion, the reaction mixture was extracted with MTBE (5V×3) and the solvent was removed from the combined organic phases by distillation. The residue was dissolved in petroleum ether (5V) and stirred at room temperature to form a precipitate. The precipitate was collected by filtration and dried to afford the titled 6-12 as a while solid (28.3% yield, 100% ee, 97.6% HPLC purity). This material was shown to be identical to 5-7 made by a pilot batch via the malic acid route in Example 1.

### Example 3: Alternate Synthesis of (S)-tert-butyl 3-allyl-4-oxopyrrolidine-1-carboxylate (5-8) Using Diastereoselective Enzymatic Reduction

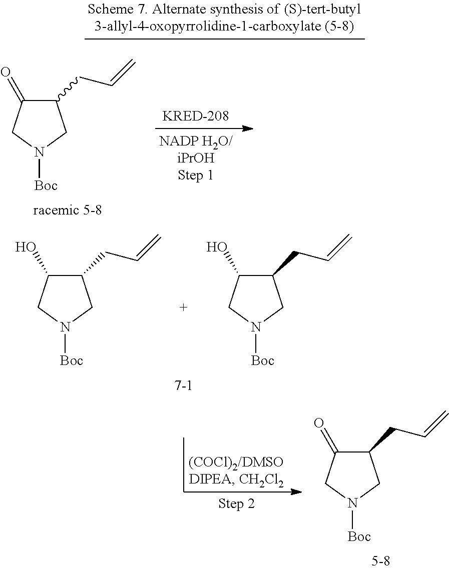 [SMILES: C=CCC1CN(C(=O)OC(C)(C)C)CC1=O ; C=CC[C@@H]1CN(C(=O)OC(C)(C)C)C[C@@H]1O ; C=CC[C@H]1CN(C(=O)OC(C)(C)C)C[C@@H]1O ; C=CC[C@H]1CN(C(=O)OC(C)(C)C)CC1=O]

Step 1: Cis/trans-tert-butyl 3-allyl-4-hydroxypyrrolidine-1-carboxylate (7-1). The NADP stock solution was prepared by adding 100 mL of 0.1 M pH 9 K₂HPO₄ solution to 20 mg of NADP, followed by 400 μL of 1M aqueous MgSO₄. The KRED/NADP solution was prepared by adding 20 mL of NADP stock solution to 80 mg of KRED-208 (4 g/L KRED) (Codexis). To 10 mL of the KRED/NADP stock solution was added 2 g of racemic 5-8 in 10 mL of iPrOH to give a tan solution. The pH was measured to be 9. The solution was heated to 40° C. After 24h, the reaction was extracted with 40 mL of EtOAc. The organic phase was washed twice with water (10 mL total) and the organic layer was concentrated to dryness to give 7-1 (1.81 g, 90% yield, in 97:3 ratio favoring the (3S,4R)/trans isomer over the (3R,4R)/cis isomer. Immediate separation of diastereoisomers was not needed as diastereomeric enrichment occurs through successive crystallizations in later steps.

Step 2: (S)-tert-butyl 3-allyl-4-oxopyrrolidine-1-carboxylate (5-8). The titled compound was oxidized according the same procedure as described in Step 8 of Example 1.

### Example 4: Synthesis of (6aS,9aR)-9a-amino-8-((S)-2-aminopropanoyl)-3-ethoxyoctahydro-[1,2]oxaborocino[7,6-c]pyrrol-1(3H)-one (8-5)

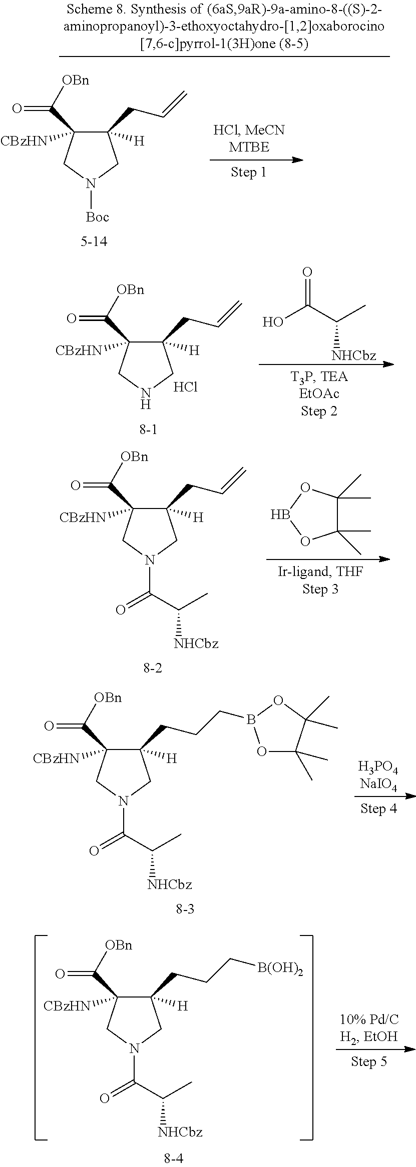

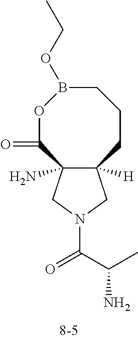 [SMILES: CCOB1CCC[C@H]2CN(C(=O)[C@H](C)N)C[C@@]2(N)C(=O)O1]

Step 1: Benzyl (3R,4S)-4-allyl-3-(((benzyloxy)carbonyl)amino)pyrrolidine-3-carboxylate hydrochloride (8-1). To a jacketed reaction vessel containing 3-benzyl 1-(tert-butyl) (3R,4S)-4-allyl-3-(((benzyloxy)carbonyl)amino)pyrrolidine-1,3-dicarboxylate (5-14) (66.1 Kg, 134 mol) under a nitrogen atmosphere, was charged acetonitrile (MeCN) (396 L) and methyl tert-butyl ether (MTBE) (463 L). The white suspension was then stirred and subsequently cooled to 0-10° C. Then, 37% HCl in water (87.8 L, 1,050 mol) was added to the reaction mixture while maintaining the temperature range of 0-10° C. Once the addition was complete, the vessel was warmed to 15-25° C. and the reaction mixture was stirred for 8 hrs. Next, MTBE (463 L) was added to the reaction suspension and the vessel was subsequently cooled to 5-15° C. After stirring for 30 min at 5-15° C., the suspension was filtered, washing the vessel and filter cake with MTBE (132 L). The white solids were then dried at 40° C. to give 56.2 kg (97%) of 8-1. ¹H NMR (400 MHz, DMSO) δ 9.93 (s, 2H), 8.53 (s, 1H), 7.45-7.28 (m, 10H), 5.74-5.54 (m, 1H), 5.17 (s, 2H), 5.13-4.96 (m, 4H), 3.95 (d, J=12.5 Hz, 1H), 3.48-3.27 (m, 2H), 2.97 (dd, J=11.6, 7.8 Hz, 1H), 2.60-2.52 (m, 1H), 2.26-2.11 (m, 1H), 1.83-1.60 (m, 1H). ¹³C NMR (126 MHz, DMSO) δ 170.32, 156.62, 136.80, 135.76, 135.32, 128.89, 128.87, 128.77, 128.49, 128.39, 118.29, 67.48, 66.95, 66.48, 52.66, 47.50, 44.92, 32.10. HRMS (ESI⁺): [M+H]⁺ calc. m/z=395.1965, found m/z=395.1998.

Step 2: Benzyl (3R,4S)-4-allyl-1-(((benzyloxy)carbonyl)-L-alanyl)-3-(((benzyloxy)carbonyl)amino)pyrrolidine-3-carboxylate (8-2). To a vessel containing benzyl (3R,4S)-4-allyl-3-(((benzyloxy)carbonyl)amino)pyrrolidine-3-carboxylate HCl (56.2 kg, 130 mol, 8-1) and N-carbobenzyloxy-L-alanine (30.6 kg, 137 mol), under nitrogen, was charged ethyl acetate (EtOAc) (562 L). The suspension was stirred and subsequently cooled to 0-10° C. Next, triethylamine (63.3 L, 454 mol) was charged to the reaction vessel, followed by 50% 1-propanephosphonic anhydride solution (T₃P) in ethyl acetate (133 L, 189 mol), while maintaining a batch temperature of 0-10° C. The reaction mixture was then warmed to 10-20° C. and agitated for 1h. Next, the suspension was cooled to 0-10° C. and H₂O (169 L) was added to the reaction vessel. The biphasic mixture was then warmed to 20-30° C. and stirred for 10 min. After phase separation, the aqueous layer was collected and the organics were washed twice with H₂O (169 L). The combined aqueous phases were then back-extracted with 10V of EtOAc (562 L). Next, the organic phases were combined and solvent exchanged into isopropanol by vacuum distillation. The concentrate was diluted to 843 L (15V) of isopropanol. The suspension was then heated to 75-85° C. and held until the solids dissolved. Then, (15V) of H₂O (843 L) was added to the solution while maintaining a temperature of 70-80° C. At the end of the addition, the solution was cooled to 10-20° C. over 2 hrs. After stirring at 10-20° C. for an additional 2 hrs, the suspension was filtered. The vessel and wet cake were rinsed with (5V) of H₂O (281 L), followed by 281 L of n-heptane. The white solids were then dried at 50° C. to yield 74.9 kg (96%) of 8-2. ¹H NMR (400 MHz, DMSO) (rotamers present) δ 8.33 (d, J=30.9 Hz, 1H), 7.54 (d, J=7.7 Hz, 1H), 7.41-7.20 (m, 15H), 5.77-5.55 (m, 1H), 5.20-4.87 (m, 8H), 4.42-4.04 (m, 2H), 3.77-3.42 (m, 2H), 3.40-3.34 (m, 1H), 2.95 (dd, J=12.0, 8.4 Hz, 1H), 2.49-2.36 (m, 1H), 2.38-2.11 (m, 1H), 1.73-1.43 (m, 1H), 1.15 (dd, J=16.8, 6.9 Hz, 3H). ¹³C NMR (rotamers present) (126 MHz, DMSO) δ 170.91, 170.84, 170.69, 170.65, 156.53, 156.46, 156.02, 137.53, 137.51, 137.05, 136.98, 136.09, 135.95, 135.80, 128.84, 128.79, 128.69, 128.63, 128.59, 128.41, 128.32, 128.30, 128.23, 128.13, 128.11, 117.71, 117.35, 67.08, 66.91, 66.62, 66.29, 66.20, 65.82, 65.77, 65.16, 55.29, 55.18, 48.94, 48.88, 48.19, 48.03, 45.35, 43.15, 33.14, 32.91, 17.68, 17.04. HRMS (ESI⁺): [M+H]⁺ calc. m/z=600.2704, found m/z=600.2707.

Step 3: Benzyl (3R,4S)-1-(((benzyloxy)carbonyl)-L-alanyl)-3-(((benzyloxy)carbonyl)amino)-4-(3-(4,4,5,5-tetramethyl-1,3,2-dioxaborolan-2-yl)propyl)pyrrolidine-3-carboxylate (8-3). To a N₂ inerted reaction vessel containing anhydrous THF (60.0 L) at 0-10° C. was added [IrCl(cod)]₂ (211 g, 0.314 mol). The mixture was stirred to dissolve all solids and then 1,2-bis(diphenylphosphanyl)ethane (DPPE) (249 g, 0.625 mol) was added to the catalyst solution. After stirring for 1h, pinacol borane (4.77 L, 32.9 mol) was added, followed by stirring for an additional 10 min. Then, the catalyst solution was warmed to ambient temp and added to a glass lined 500 L vessel containing a suspension of benzyl (3R,4S)-4-allyl-1-(((benzyloxy)carbonyl)-L-alanyl)-3-(((benzyloxy)carbonyl)amino)pyrrolidine-3-carboxylate (8-2) (75.3 kg, 126 mol), pinacol borane (18.9 L, 131 mol) and anhydrous THF (240 L). After stirring for 4 hrs, the reaction was deemed complete by HPLC and the reaction solution was cooled to 0-10° C. Next, 15% aq. N-acetyl cysteine (NAC) (376 L) was added. The biphasic mixture was heated to 45-55° C. and stirred for 30 min. Then, after cooling to 15-25° C., MTBE (376 L) and aq. 13% sodium carbonate (376 L) were added sequentially. The biphasic mixture was allowed to stir for 15 min, followed by phase separation and removal of the aqueous layer. Then, 15% NAC (376 L) was added to the organic layer and the mixture was heated to 45-55° C. After stirring for 30 min, the mixture was cooled to 15-25° C. and 13% aq. sodium carbonate (376 L) was added. The biphasic mixture was then allowed to stir for 15 min, followed by phase separation. Next, the organic phase was washed with a brine solution and the organic phase was charged to a reaction vessel containing C-941 charcoal (7.53 kg). Stirring began and the suspension was heated to 45-55° C. After 2 hrs, the suspension was cooled to 15-20° C. and filtered. The organic filtrate was then concentrated and solvent swapped with 329 L of THF. Next, n-heptane (602 L) was slowly added and the subsequent suspension was stirred for 10 hrs. Next, additional n-heptane (151 L) was added to the crystallization vessel. After stirring for an additional 3 hrs at room temperature, the white suspension was filtered, rinsed, and dried to yield 8-3 as a white solid (82.9 kg, 92.4%). ¹H NMR (500 MHz, DMSO) (rotamers present) δ 8.23 (d, J=39.7 Hz, 1H), 7.52 (dd, J=14.8, 7.6 Hz, 1H), 7.41-7.12 (m, 15H), 5.24-4.80 (m, 6H), 4.42-4.02 (m, 2H), 3.89-3.66 (m, 1H), 3.67-3.55 (m, 1H), 3.38 (d, J=12.8 Hz, 1H), 2.94 (dd, J=11.8, 8.5 Hz, 1H), 2.46-2.25 (m, 1H), 1.56-0.97 (m, 17H), 0.97-0.76 (m, 1H), 0.71-0.40 (m, 2H). ¹³C NMR (126 MHz, DMSO) (rotamers present) δ 170.92, 170.80, 156.53, 156.03, 137.51, 137.02, 136.07, 135.89, 128.82, 128.78, 128.63, 128.50, 128.43, 128.38, 128.36, 128.26, 128.22, 128.17, 128.12, 83.09, 67.48, 67.05, 66.97, 66.78, 66.22, 66.11, 65.80, 65.77, 65.48, 55.38, 55.26, 49.22, 47.99, 46.04, 43.74, 31.42, 31.30, 25.13, 25.09, 25.03, 24.99, 22.56, 22.47, 17.81, 17.02, 11.33. HRMS (ESI⁺): [M+H]⁺ calc. m/z=728.3712, found m/z=728.3721.

Steps 4 and 5: (6aS,9aR)-9a-amino-8-((S)-2-aminopropanoyl)-3-ethoxyoctahydro-[1,2]oxaborocino[7,6-c]pyrrol-1(3H)-one (8-5). To a N₂ inerted 500 gal vessel was charged benzyl (3R,4S)-1-(((benzyloxy)carbonyl)-L-alanyl)-3-(((benzyloxy)carbonyl)amino)-4-(3-(4,4,5,5-tetramethyl-1,3,2-dioxaborolan-2-yl)propyl)pyrrolidine-3-carboxylate (8-3) (82.9 kg, 113.9 mol) followed by THF (368.5 kg). The mixture was agitated at 20±5° C. until a solution formed. Next, 6.7% aqueous phosphoric acid (427.8 kg) followed by sodium periodate (29.3 kg) were added to the reactor and the mixture was agitated for NLT 8 hrs. Upon confirmation of the removal of the pinacol ester, water (415 L) and MTBE (306.7 kg) were added and the mixture was agitated for about 10 minutes. Agitation was stopped and upon settling, the lower level was removed from the reactor. Next the organic layer was washed twice with, THF (73.7 kg) and 5% ascorbic acid (422.0 kg). The organic layer was then washed with USP water until pH of the solution was above 5. Upon removal of the aqueous layer, the mixture was solvent exchanged into ethanol and transferred to an inerted hydrogenation vessel containing 83 L of water and 4.1 kg of 10% Pd/C. The mixture was then hydrogenated at 50 PSIG at 20±5° C. until less than 1% of starting material and intermediates remained by HPLC. Upon completion, the reactor was inerted and the catalyst removed by filtration. The reactor and solids were then rinsed with ethanol. To the inerted, rinsed reactor was charged 8.30 kg of C-941 charcoal and the filtered ethanol solution was recharged to the reaction vessel. The mixture agitated for NLT 30 min at 20±5° C. Next the mixture was filtered and the solids were washed with ethanol. All of the filtrates were combined and transferred to a clean reactor. Next, water was azeotropically removed through distillation with ethanol until KF of the mixture was below 1%, whereupon the product gradually precipitated in the reactor. The mixture was then heated to reflux and gradually cooled to −5±5° C. over 8 hrs. The mixture was then stirred at −5±5° C. for 12h and filtered. The isolated drug substance was then rinsed with cold line filtered ethanol and then dried at NMT 50° C. to yield 27 kg (80%) of 8-5. ¹H NMR (500 MHz, DMSO) (rotamers present) δ 7.20-6.57 (m, 2H), 3.87-3.76 (m, 1H), 3.72-3.58 (m, 1H), 3.57-3.18 (m, 4H), 3.18-2.76 (m, 1H), 2.47-2.22 (m, 1H), 1.90-1.62 (m, 3H), 1.50-1.36 (m, 1H), 1.14-1.04 (m, 6H), 1.05-0.90 (m, 1H), 0.74-0.61 (m, 1H), 0.50-0.35 (m, 1H). ¹³C NMR (126 MHz, DMSO) (rotamers present) δ 174.49, 174.18, 172.81, 172.61, 66.52, 66.44, 65.24, 65.19, 57.03, 56.96, 51.90, 51.72, 49.28, 49.12, 49.08, 49.05, 48.02, 47.98, 47.58, 46.97, 40.50, 40.43, 40.34, 40.28, 40.17, 40.10, 40.00, 39.84, 39.67, 39.50, 27.40, 27.28, 27.04, 26.92, 23.47, 23.26, 23.23, 21.97, 21.38, 19.03, 18.66; MS (ESI⁺): [M-CH₃CH₂O]⁺ calc. m/z=252.15, found m/z=252.11.

### Example 5: Alternate Route to (6aS,9aR)-9a-amino-8-((S)-2-aminopropanoyl)-3-ethoxyoctahydro-[1,2]oxaborocino[7,6-c]pyrrol-1(3H)-one (8-4)

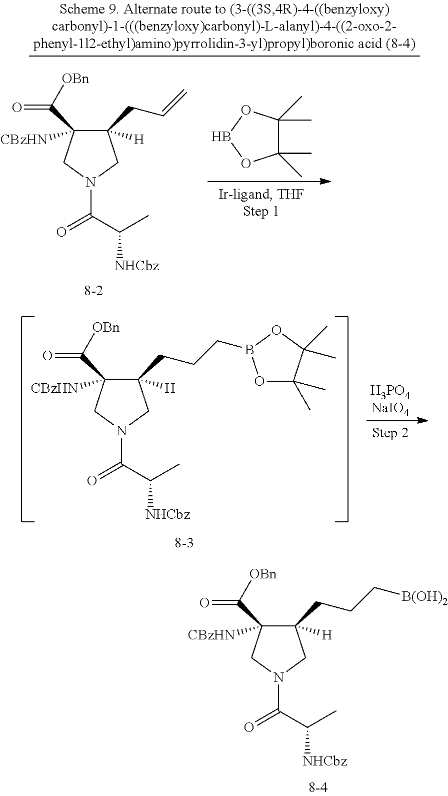

Step 1: To a 500 mL 3-neck round bottom flask equipped with a mechanic stirrer and J-KEM thermal controller was charged chloro-1,5-cyclooctadiene iridium (I) dimer (0.176 g, 0.262 mmol), 1,2-bis(diphenylphosphanyl)ethane (0.208 g, 0.523 mmol) and THF (78.5 mL, 5V). The bright orange/yellow solution was agitated for 10 min and 4,4,5,5-tetramethyl-1,3,2-dioxaborolane (4.35 g, 34.0 mmol) was charged (18.3-20.7° C.). The light green solution was agitated for 30 min and 8-2 (15.69 g, 26.2 mmol) was charged portion wise (3.92 g x 4) over 10 min maintaining 20±5° C. Following 1.5 h at rt the reaction was complete by HPLC (<2% SM).

Step 2: To the above solution was added a solution of 4.0N HCl in water (78.5 mL, 5V) maintaining 20±5° C. (the solution became cloudy after addition of 20 mL of HCl solution). The reaction mixture was stirred at rt for 1h, then NaIO₄ solid was added (11.19 g, 52.3 mmol; exotherm, maintaining <30° C.). The reaction mixture was allowed to stir at rt for 3h, water (5V) and IPAc (10V) was added. The mixture was partitioned between IPAc and water. The aqueous layer was extracted with IPAc (10V). The combined organics were washed with 10% sodium thiosulfate aqueous solution (15V), dried over MgSO₄, filtered and distilled in vacuum to ˜10.0 volumes. Activated charcoal (3.38 g, 20 wt %) was charged to the mixture and stirred at 45° C. (oil bath) for 1h. The mixture was filtered through a pad of celite with IPAc washing (3V) to give light yellow solution (total volumes: 13V). Crystallization was performed by slow addition of heptane (15V) at rt (solution became cloudy after addition of 30 mL Heptane). The mixture was stirred at rt for 30 minutes after addition of heptane. Filtration gave solid product (ML was used to transfer solid product to the filtration funnel). The resultant white solid was dried in a vacuum oven with N₂ sweep at 40° C. overnight to afford the titled 8-4 (15.39 g, 91% yield, 94.7% pure @ 220 nm).

### Example 6: Alternate Route A to (6aS,9aR)-9a-amino-8-((S)-2-aminopropanoyl)-3-ethoxyoctahydro-[1,2]oxaborocino[7,6-c]pyrrol-1(3H)-one (8-5)

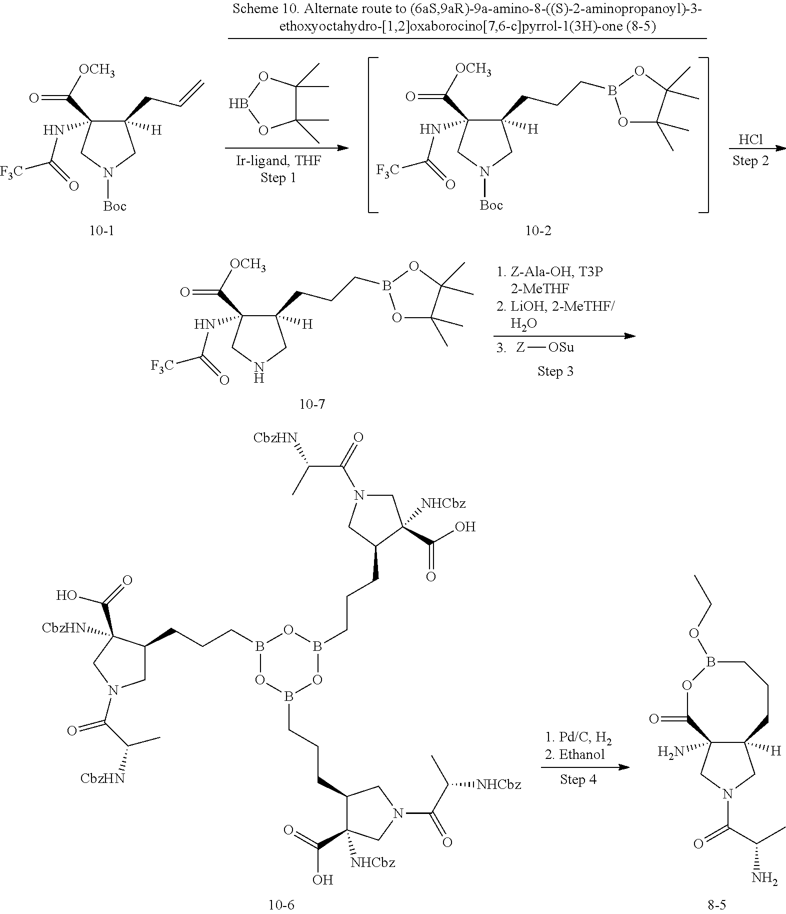

Steps 1 and 2: Methyl (3R,4S)-4-(3-(4,4,5,5-tetramethyl-1,3,2-dioxaborolan-2-yl)propyl)-3-(2,2,2-trifluoroacetamido)pyrrolidine-3-carboxylate hydrochloride (10-7). To a 250 mL jacketed reactor with a jacket temp of 20° C. was charged chloro-1,5-cyclooctadiene iridium (I) dimer (0.141 g, 0.210 mmol), 1,2-bis(diphenylphosphanyl)ethane (0.184 g, 0.463 mmol) and dichloromethane (80 mL, 5V). The bright orange/yellow solution was agitated for 20 min and 4,4,5,5-tetramethyl-1,3,2-dioxaborolane (7.32 ml, 50.5 mmol) was charged maintaining the temperature. The pale yellow solution was agitated for 30 min and 10-1 (16 g, 42.1 mmol) was charged dropwise over 30 min as a solution in dichloromethane (80 mL, 5V) maintaining 20±5° C. Following 3 h at that temperature, the reaction was noted as complete by HPLC and was quenched by the addition of water (80 mL, 5V). The biphasic mixture was allowed to stir for 30 min. Agitation was ceased and the layers were separated. The organics layer was dried over sodium sulfate, filtered and distilled using house vacuum to ca. 2.0 volumes. Dioxane (64 mL, 4V) was charged to the mixture and distillation was continued to ˜2.0 volumes with a max jacket temperature of ˜55° C. The reaction mixture was cooled to 20° C. and 4N hydrochloric acid in dioxane (42.1 ml, 168 mmol) was charged with no exotherm observed. Following 15h at 23° C., the reaction was complete by HPLC and was diluted with MTBE (320 mL, 20V). Crystallization was initiated by addition of seed crystals (1 mg) and within 5 min a thick slurry was present. The reaction mixture was cooled to ˜10° C. over 15 min, agitated for 1h and filtered, rinsing with MTBE (2×50 mL). The resultant white solid was dried in a vacuum oven at 50° C. until constant weight to afford 10.7 (16.77 g, 90% yield, 95% purity). ¹H NMR (400 MHz, DMSO) δ 10.38 (s, 1H), 9.56 (s, 2H), 3.96 (d, J=12.8 Hz, 1H), 3.70 (s, 3H), 3.60-3.52 (m, 1H), 3.34 (d, J=12.8 Hz, 1H) 2.90 (t, J=10.9 Hz, 1H), 2.73-2.62 (m, 1H), 1.55-1.40 (m, 1H), 1.37-1.27 (m, 2H), 1.18 (s, 12H), 1.03-0.81 (m, 1H), 0.69 (t, J=7.7 Hz, 2H).

Steps 3: (3R,3′R,3″R,4S,4′S,4″S)-4,4′,4″-((1,3,5,2,4,6-trioxatriborinane-2,4,6-triyl)tris(propane-3,1-diyl))tris(1-(((benzyloxy)carbonyl)-L-alanyl)-3-(((benzyloxy)carbonyl)amino)pyrrolidine-3-carboxylic acid) (10-6). To a 500 mL jacketed reactor, triethylamine (20.06 ml, 144 mmol) was charged to a cold solution of 10-7 (16 g, 36.0 mmol) in 2-MeTHF (96 mL, 6 vol) maintaining a temperature of <5° C. The heterogeneous mixture was agitated for 20 min and Z-L-alanine (8.84 g, 39.6 mmol) was charged as a solid followed by dropwise addition of 1-propanephosphonic acid cyclic anhydride in 2-MeTHF (26.4 ml, 43.2 mmol) maintaining <5° C. (highly exothermic). Following 1h at 0° C., the reaction was noted as complete by HPLC, quenched by the addition of water (80 mL) and allowed to warm to rt. The layers were separated and the aqueous layer was extracted with 2-MeTHF (32 mL, 2 vol). The combined organic layers were washed with half saturated brine (64 mL). Next, lithium hydroxide monohydrate (6.04 g, 144 mmol) as a solution in water (160 mL) was charged to the organic layer (mild exotherm). The reaction mixture was warmed at 35° C. for 24h with vigorous agitation and whereupon the reaction was deemed complete by HPLC. The reaction mixture pH was then adjusted to ˜2 by the addition of concentrated HCl (12 mL) and diluted with MTBE (80 mL, 5 vol). Next, phenylboronic acid (4.39 g, 36.0 mmol) was charged as a solid. The reaction mixture was agitated for 16 h at 23° C. to complete the deprotection of the pinacol boronic ester and the layers were separated. The organic layer was extracted with 1N HCl (100 mL) and the combined aqueous layers were washed with additional MTBE (100 mL) containing phenylboronic acid (0.439 g, 3.6 mmol) to facilitate removal of pinacol from solution. The layers were separated and the aqueous layer containing product was adjusted to pH 9.0 by addition of 3N NaOH. Acetonitrile (80 mL) was added followed by benzyl (2,5-dioxopyrrolidin-1-yl) carbonate (9.86 g, 39.6 mmol) as a solid. The pH of the reaction was maintained between 8 and 10 by the addition of 1N NaOH. After 1h an additional charge of benzyl (2,5-dioxopyrrolidin-1-yl) carbonate (Z-OSu, 0.986 g, 3.96 mmol) was performed and the pH was continuously adjusted to maintain ˜8. The reaction was deemed complete by HPLC analysis after an additional 1h and the pH was adjusted to ˜3 facilitated by the addition of concentrated HCl. The reaction mixture was extracted with isopropyl acetate (3×300 mL) and the combined organic extracts were washed with brine (3×100 mL), dried over sodium sulfate and filtered to afford the crude monomer in isopropyl acetate. The reaction mixture was distilled under atmospheric pressure with a jacket temperature of 100° C. to a volume of 600 mL to remove water. Isopropyl acetate (100 mL) was charged and the distillation continued until a reaction volume of 600 mL was reached. During distillation the reaction mixture became thick with solids and was cooled to 0° C. over 1h. The mixture was then filtered, washed with isopropyl acetate (2×40 mL) and the wet solid was dried in a vacuum oven at 50° C. to afford 10-6 (13.52 g, 70% yield, 98.9% pure) as a white solid. ¹H NMR (500 MHz, DMSO) (rotamers present) δ 8.03 (d, J=35.8 Hz, 1H), 7.51 (dd, J=19.3, 7.7 Hz, 1H), 7.41-7.26 (m, 10H), 5.13-4.88 (m, 4H), 4.41-4.03 (m, 2H), 3.88-3.67 (m, 1H), 3.64-2.87 (m, 3H), 2.43-2.20 (m, 1H), 1.66-0.81 (m, 7H), 0.70-0.43 (m, 2H).

Step 4: (3R,3′R,3″R,4S,4′S,4″S)-4,4′,4″-((1,3,5,2,4,6-trioxatriborinane-2,4,6-triyl)tris(propane-3,1-diyl))tris(1-(((benzyloxy)carbonyl)-L-alanyl)-3-(((benzyloxy)carbonyl)amino)pyrrolidine-3-carboxylic acid) (10-6) was used to prepare 8-5 according the same procedure as in Step 5 of Example 4.

### Example 7: Alternate Route B to (6aS,9aR)-9a-amino-8-((S)-2-aminopropanoyl)-3-ethoxyoctahydro-[1,2]oxaborocino[7,6-c]pyrrol-1(3H)-one (8-5)

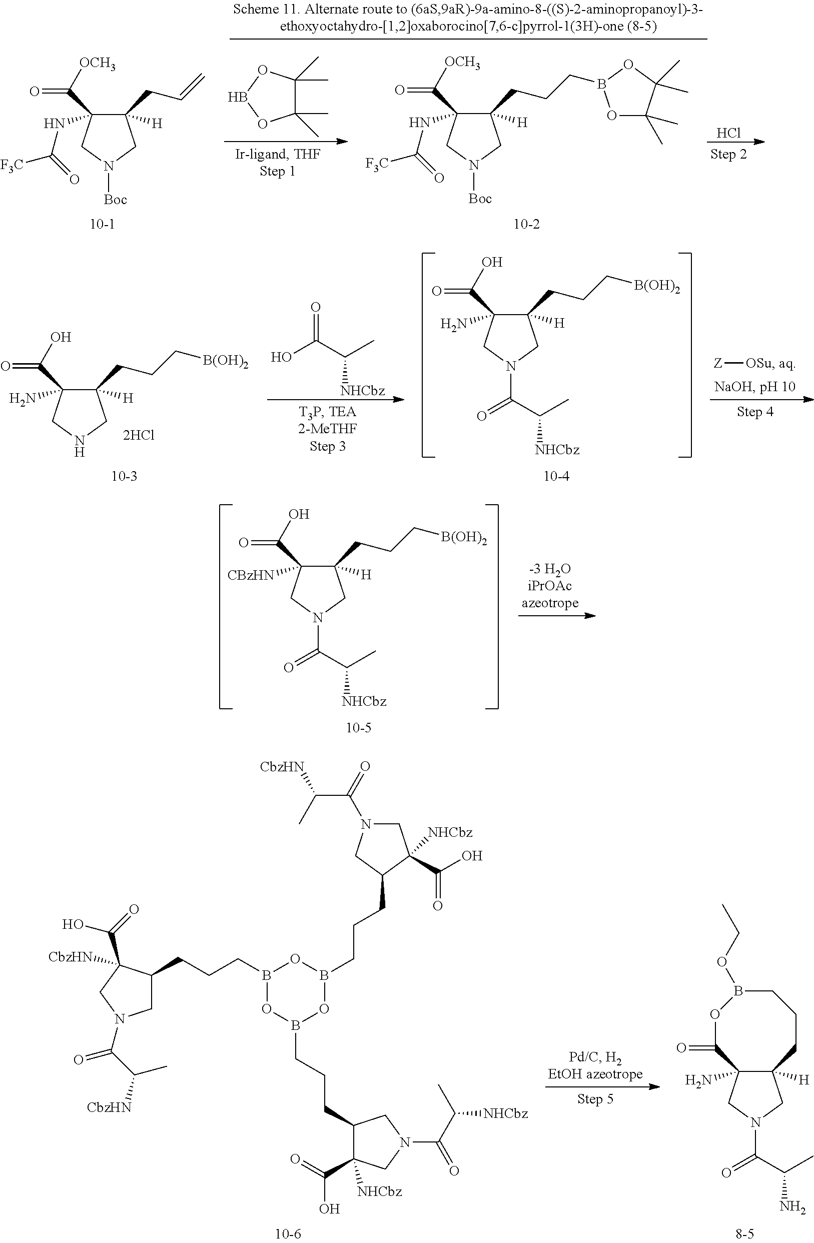

Step 1: 1-(tert-Butyl) 3-methyl (3R,4S)-4-(3-(4,4,5,5-tetramethyl-1,3,2-dioxaborolan-2-yl)propyl)-3-(2,2,2-trifluoroacetamido)pyrrolidine-1,3-dicarboxylate (10-2) was prepare according to the same procedure as in Step 1 of Example 6.

Step 2: (3R,4S)-3-amino-4-(3-boronopropyl)pyrrolidine-3-carboxylic acid dihydrochloride (10-3). To a slurry of 10-2 (5 g, 9.84 mmol) in water (15 mL, 5V) was charged hydrochloric acid (8.13 mL, 98 mmol, 10 eq) and the resultant slurry was agitated at 75-80° C. while distilling low boiling organics. Upon reaction completion (8h), the mixture was azeotroped with dioxane (100 mL) to remove excess water and HCl maintaining to ˜3V. Acetonitrile (15 mL, 3v) was then charged and the mixture was agitated until filterable solids formed. The solids were collected by filtration, washed with dioxane, and dried in vacuo at 50° C. to afford 10-3 as a white solid (1.52 g, 54%). ¹H NMR (400 MHz, D₂O) δ 3.95 (dd, J=12.8, 2.5 Hz, 1H), 3.78 (dd, J=11.8, 8.4 Hz, 1H), 3.55-3.47 (m, 1H), 3.29 (t, J=11.7 Hz, 1H), 2.61 (tdd, J=11.5, 8.3, 3.7 Hz, 1H), 1.66 (dddd, J=13.2, 10.2, 6.0, 3.6 Hz, 1H), 1.48-1.18 (m, 3H), 0.83-0.62 (m, 2H).

Step 3 and 4: (3R,3′R,3″R,4S,4′S,4″S)-4,4′,4″-((1,3,5,2,4,6-trioxatriborinane-2,4,6-triyl)tris(propane-3,1-diyl))tris(1-(((benzyloxy)carbonyl)-L-alanyl)-3-(((benzyloxy)carbonyl)amino)pyrrolidine-3-carboxylic acid) (10-6). A solution of 10-3 (13 g, 60 mmol, 1 eq) in water (15V, 195 mL) was adjusted to pH 9-10 with sodium hydroxide (50%, aq) at 0° C. To the solution was added 2,5-dioxopyrrolidin-1-yl ((benzyloxy)carbonyl)-L-alaninate (38.8 g, 120 mmol, 2 eq) and agitated at 0° C. (˜3h) until complete by HPLC (CAD). The reaction mixture was acidified to pH 3-3.5 by addition of hydrochloric acid (conc., aq) and then washed with toluene (5V) and ethyl acetate (2×5V). The aqueous layer was adjusted to pH 10-11 by the addition of sodium hydroxide (25%) and a solution of benzyl (2,5-dioxopyrrolidin-1-yl) carbonate (21.13 g, 85 mmol) in acetonitrile (5V, 60 mL) was added. The pH was maintained at 10-11 by continuous addition of sodium hydroxide (25%) at rt until the reaction was complete by HPLC (˜2h). The reaction mixture was acidified to pH 3-4 by addition of hydrochloric acid (conc., aq) and extracted with EtOAc (10V). The organics were washed with brine (3×1V) and distilled (atm.) at a constant volume while charging ethyl acetate (20V) until a thick while precipitate was present. The slurry was cooled to rt, agitated for 16h and filtered, washing with ethyl acetate (2V). The solids were then dried to afford 10-6 as a white solid (20.78 g, 64% yield). ¹H NMR (500 MHz, DMSO) (rotamers present) δ 8.03 (d, J=35.8 Hz, 1H), 7.51 (dd, J=19.3, 7.7 Hz, 1H), 7.41-7.26 (m, 10H), 5.13-4.88 (m, 4H), 4.41-4.03 (m, 2H), 3.88-3.67 (m, 1H), 3.64-2.87 (m, 3H), 2.43-2.20 (m, 1H), 1.66-0.81 (m, 7H), 0.70-0.43 (m, 2H).

Step 5: (3R,3′R,3″R,4S,4′S,4″S)-4,4′,4″-((1,3,5,2,4,6-trioxatriborinane-2,4,6-triyl)tris(propane-3,1-diyl))tris(1-(((benzyloxy)carbonyl)-L-alanyl)-3-(((benzyloxy)carbonyl)amino)pyrrolidine-3-carboxylic acid) (10-6) was used to prepare 8-5 according the same procedure as in Step 5 of Example 4.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the following claimed invention(s).

1. A process for preparing a compound of formula 21 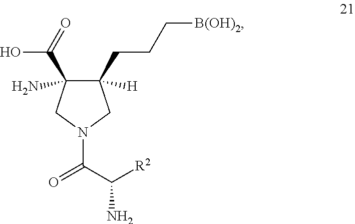
or a pharmaceutically acceptable salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 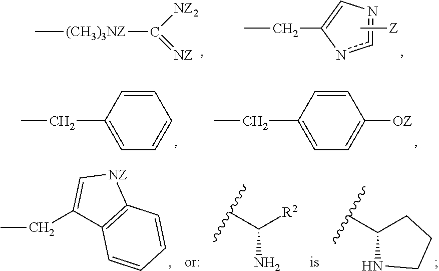
and
each Z is independently H, (C₁-C₆)alkyl, or a suitable protecting group,
comprising treating a compound of formula 20 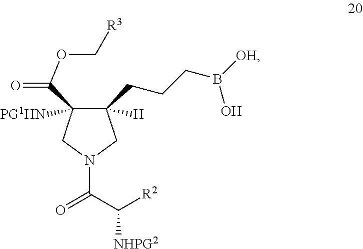
or a salt thereof, wherein:
R³ is a substituted or unsubstituted ring selected from aryl and heteroaryl; and
PG¹ and PG² are independently suitable amine protecting groups,
under suitable conditions to deprotect the compound of formula 20 to form the compound of formula 21 or a pharmaceutically acceptable salt thereof.

2. (canceled)

3. A process for preparing a compound of formula 22 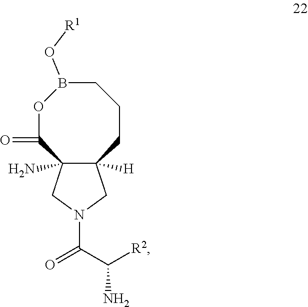
or a pharmaceutically acceptable salt thereof, wherein:
R¹ is H or (C₁-C₆)alkyl;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 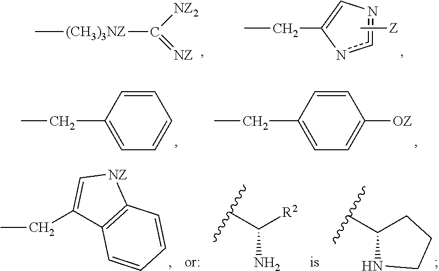
and
each Z is independently H, (C₁-C₆)alkyl, or a suitable protecting group,
comprising treating a compound of formula 20 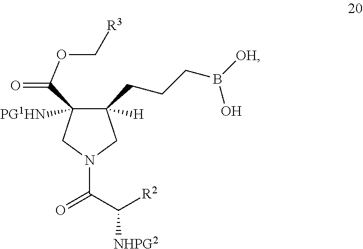
or a salt thereof, wherein:
R³ is a substituted or unsubstituted ring selected from aryl and heteroaryl; and
PG¹ and PG² are independently suitable amine protecting groups,
under suitable conditions to deprotect and condense the compound of formula 20 to form the compound of formula 22 or a pharmaceutically acceptable salt thereof.

4. (canceled)

5. A process for preparing a compound of formula 20 as defined in claim 1, the process comprising hydrolyzing the boronate of a compound of formula 19 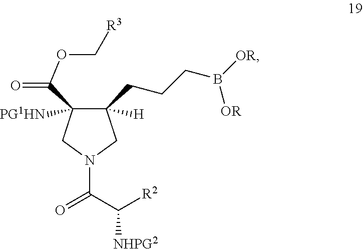
or a salt thereof, wherein:
each R is independently (C₁-C₆)alkyl, or:
two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 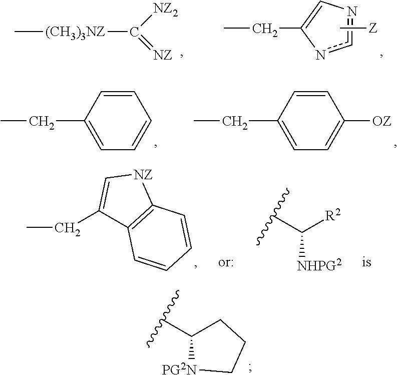
each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
under suitable conditions to hydrolyze the boronate of the compound of formula 19 to form the compound of formula 20 or a salt thereof.

6. (canceled)

7. A process for preparing a compound of formula 19 as defined in claim 5, the process comprising hydroborating a compound of formula 18 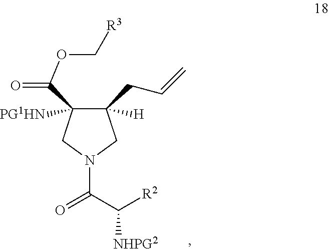
or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 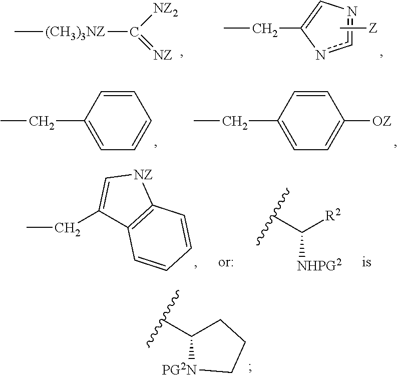
each Z is independently H, (C₁-C₆)alkyl, or a suitable protecting group;
R³ is a substituted or unsubstituted ring selected from aryl and heteroaryl; and
PG¹ and PG² are independently suitable amine protecting groups,
under suitable conditions to hydroborate the compound of formula 18 to form the compound of formula 19 or a salt thereof.

8. (canceled)

9. A process for preparing a compound of formula 18 as defined in claim 7, the process comprising coupling a compound of formula 17 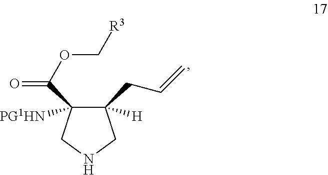
or a salt thereof, wherein:
R³ is a substituted or unsubstituted ring selected from aryl and heteroaryl; and
PG¹ is a suitable amine protecting group,
with a compound of formula 16 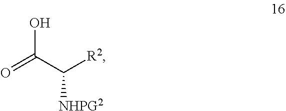
or salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 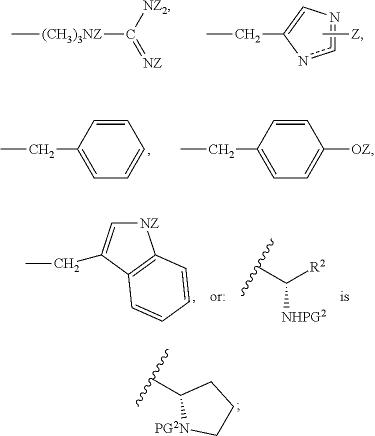
each Z is independently H, (C₁-C₆)alkyl, or a suitable protecting group; and
PG² is a suitable amine protecting group,
under amide forming conditions to form the compound of formula 18 or a salt thereof.

10. (canceled)

11. A process for preparing a compound of formula 17 as defined in claim 9, the process comprising removing the protecting group from the secondary amine of a compound of formula 15 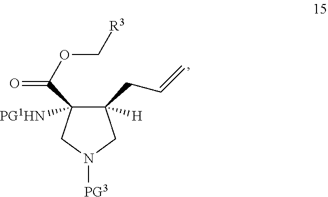
or a salt thereof, wherein:
R³ is a substituted or unsubstituted ring selected from aryl and heteroaryl; and
PG¹ and PG³ are independently suitable amine protecting groups,
under suitable conditions to remove the protecting group from the secondary amine of the compound of formula 15 to form the compound of formula 17 or a salt thereof.

12. (canceled)

13. A process for preparing a compound of formula 15 as defined in claim 11, the process comprising adding a protecting group to the primary amine of a compound of formula 14 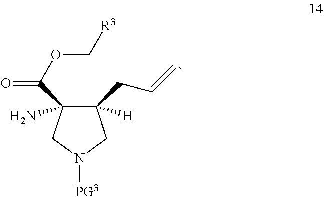
or a salt thereof, wherein:
R³ is a substituted or unsubstituted ring selected from aryl and heteroaryl; and
PG³ is a suitable amine protecting group,
under suitable conditions to add the protecting group to the primary amine of the compound of formula 14 to form the compound of formula 15 or a salt thereof.

14. (canceled)

15. A process for preparing a compound of formula 14 as defined in claim 13, the process comprising reducing the azide of a compound of formula 13 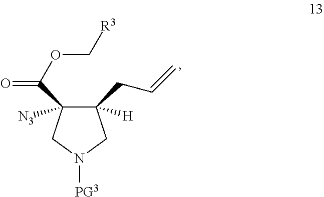
or a salt thereof, wherein:
R³ is a substituted or unsubstituted ring selected from aryl and heteroaryl; and
PG³ is a suitable amine protecting group,
under suitable conditions to reduce the azide of a compound of formula 13 to form the compound of formula 14 or a salt thereof.

16. (canceled)

17. A process for preparing a compound of formula 13 as defined in claim 15, the process comprising adding a protecting group to the carboxylic acid of a compound of formula 12 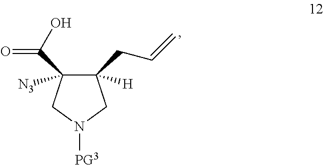
or a salt thereof, wherein:
PG³ is a suitable amine protecting group,
under suitable conditions to add a protecting group to the carboxylic acid of a compound of formula 12 to form the compound of formula 13 or a salt thereof.

18. (canceled)

19. A process for preparing a compound of formula 12 as defined in claim 17, the process comprising treating a compound of formula 11 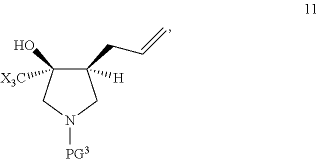
or a salt thereof, wherein:
each X is independently hydrogen or halogen; and
PG³ is a suitable amine protecting group,
under suitable conditions to convert the compound of formula 11 into the compound of formula 12 or a salt thereof.

20. (canceled)

21. A process for preparing a compound of formula 11 as defined in claim 19, the process comprising removing the protecting group from the tertiary alcohol of a compound of formula 10 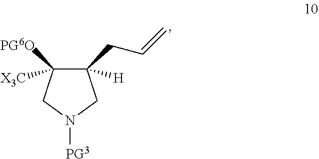
or a salt thereof, wherein:
each X is independently hydrogen or halogen;
PG³ is a suitable amine protecting group; and
PG⁶ is a suitable hydroxyl protecting group,
under suitable conditions to remove the protecting group from the tertiary alcohol of the compound of formula 10 to form the compound of formula 11 or a salt thereof.

22. (canceled)

23. A process for preparing a compound of formula 10 as defined in claim 21, the process comprising treating a compound of formula 9 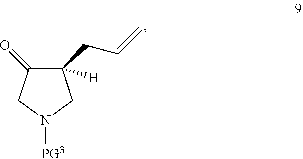
or a salt thereof, wherein:
PG³ is a suitable amine protecting group,
under suitable conditions to alkylate the compound of formula 9 to form the compound of formula 10 or a salt thereof.

24. (canceled)

25. A process for preparing a compound of formula 9 as defined in claim 23, the process comprising oxidizing the secondary alcohol of a compound of formula 8 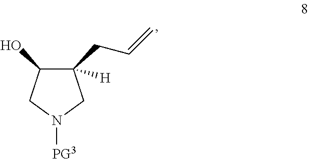
or a salt thereof, wherein:
PG³ is a suitable amine protecting group,
under suitable conditions to oxidize the secondary alcohol of a compound of formula 8 to form the compound of formula 9 or a salt thereof.

26. (canceled)

27. A process for preparing a compound of formula 8 as defined in claim 25, the process comprising removing the protecting group from the secondary alcohol and the initial protecting group (PG^{N}, if PG^{N} is a suitable amine protecting group) from the secondary amine, and adding another protecting group to the secondary amine of a compound of formula 7 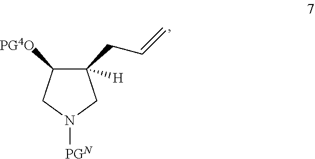
or a salt thereof, wherein:
PG⁴ is a suitable hydroxyl protecting group; and
PG^{N} is H or a suitable amine protecting group,
under suitable conditions to remove the protecting group from the secondary alcohol and the initial protecting group (PG^{N}, if PG^{N} is a suitable amine protecting group) from the secondary amine, and adding another protecting group to the secondary amine of a compound of formula 7 to form the compound of formula 8 or a salt thereof.

28. (canceled)

29. A process for preparing a compound of formula 7 as defined in claim 27, the process comprising treating a compound of formula 6 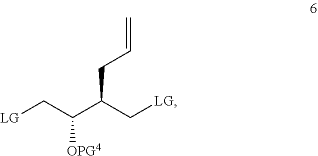
or a salt thereof, with H₂N-PG^{N}, wherein:
LG is a suitable leaving group;
PG⁴ is a suitable hydroxyl protecting group; and
PG^{N} is H or a suitable amine protecting group,
under suitable conditions to perform a double displacement reaction on the compound of formula 6 to form the compound of formula 7 or a salt thereof.

30. (canceled)

31. A process for preparing a compound of formula 6 as defined in claim 29, the process comprising converting the primary alcohols of a compound of formula 5 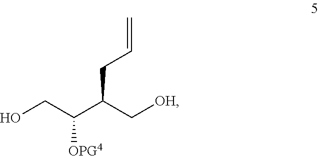
or a salt thereof, wherein:
PG⁴ is a suitable hydroxyl protecting group,
into leaving groups under suitable conditions to form the compound of formula 6 or a salt thereof.

32. (canceled)

33. A process for preparing a compound of formula 5 as defined in claim 31, the process comprising reducing the alkyl carboxylates of a compound of formula 4 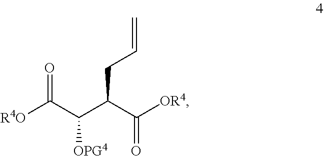
or a salt thereof, wherein:
each R⁴ is independently (C₁-C₆)alkyl;
PG⁴ is a suitable hydroxyl protecting group,
under suitable conditions to reduce the alkyl carboxylates of the compound of formula 4 to form the compound of formula 5 or a salt thereof.

34. (canceled)

35. A process for preparing a compound of formula 4 as defined in claim 33, the process comprising adding a protecting group to the secondary alcohol of a compound of formula 3 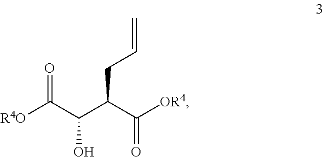
or a salt thereof, wherein:
each R⁴ is independently (C₁-C₆)alkyl;
under suitable conditions to form the compound of formula 4 or a salt thereof.

36. (canceled)

37. A process for preparing a compound of formula 3 as defined in claim 35, the process comprising allylating a compound of formula 2 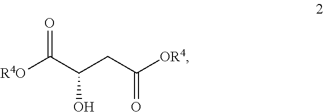
or a salt thereof, wherein:
each R⁴ is independently (C₁-C₆)alkyl;
under suitable conditions to allylate the compound of formula 2 to form the compound of formula 3 or a salt thereof.

38. (canceled)

39. A process for preparing a compound of formula 2 as defined in claim 37, the process comprising esterifying L-malic acid under suitable conditions to form the compound of formula 2 or a salt thereof.

40. (canceled)

41. A process for preparing a compound of formula 9 as defined in claim 23, the process comprising oxidizing the secondary alcohol of a compound of formula 8b 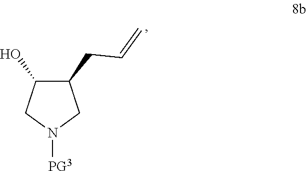
or a salt thereof, wherein:
PG³ is a suitable amine protecting group,
under suitable conditions to form the compound of formula 9 or a salt thereof.

42. (canceled)

43. A process for preparing a compound of formula 8b as defined in claim 41, the process comprising diastereoselective enzymatic reduction of a compound of formula 7b 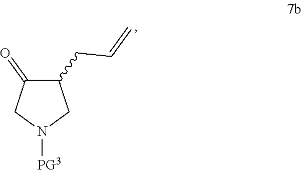
or a salt thereof, wherein:
PG³ is a suitable amine protecting group,
under suitable conditions to form the compound of formula 8b or a salt thereof.

44-63. (canceled)

64. A compound of formula 20 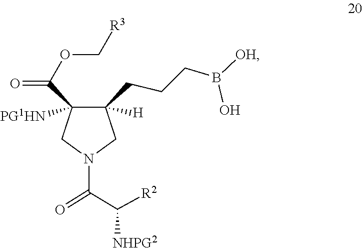
or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 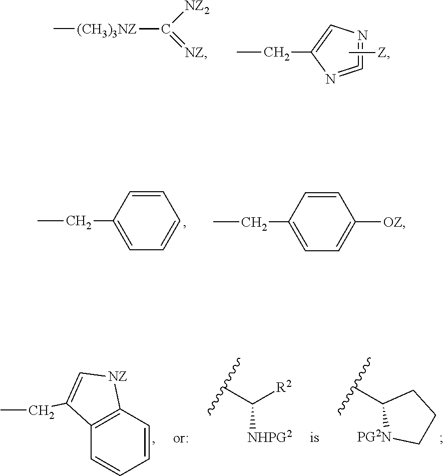
each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

65. (canceled)

66. A compound of formula 19 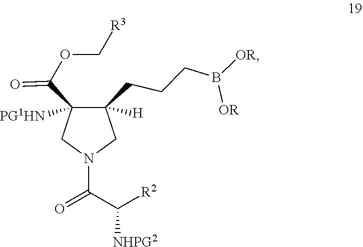
or a salt thereof, wherein:
each R is independently (C₁-C₆)alkyl, or:
two R groups are optionally taken together with their intervening atoms to form a substituted or unsubstituted monocyclic or bicyclic ring selected from a saturated or partially unsaturated heterocycle;
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 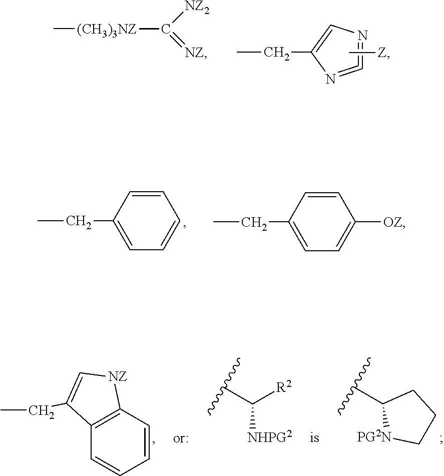
each Z is independently H, (C₁-C₆)alkyl, or a suitable protecting group;
R³ is a substituted or unsubstituted ring selected from aryl and heteroaryl: and
PG¹ and PG² are independently suitable amine protecting groups.

67. (canceled)

68. A compound of formula 18 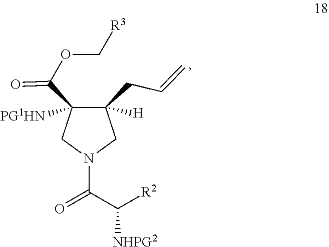
or a salt thereof, wherein:
R² is H, (C₁-C₆)alkyl, —CH₂OZ, —CH(CH₃)OZ, —CH₂SZ, —(CH₂)₂SCH₃, —CH₂CONZ₂, —(CH₂)₂CONZ₂, —CH₂CO₂Z, —(CH₂)₂CO₂Z, —(CH₂)₄NZ₂, 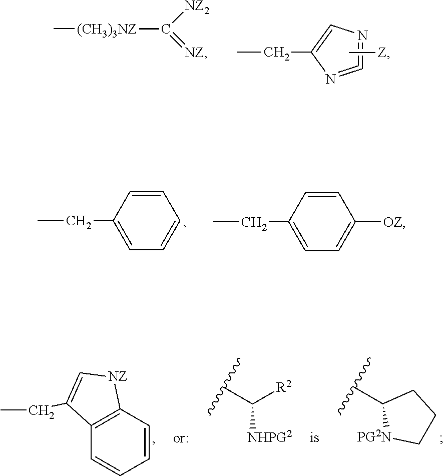
each Z is independently H, (C₁-C₆)alkyl, or a suitable protecting group;
R³ is a substituted or unsubstituted ring selected from aryl and heteroaryl; and
PG¹ and PG² are independently suitable amine protecting groups.

69. (canceled)

70. A compound of formula 17 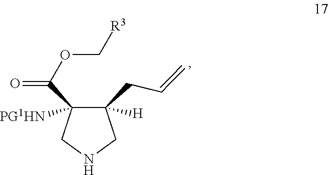
or a salt thereof, wherein:
R³ is a substituted or unsubstituted ring selected from aryl and heteroaryl; and
PG¹ is a suitable amine protecting group.

71. (canceled)

72. A compound of formula 15 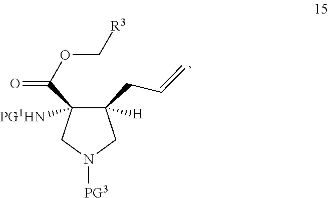
or a salt thereof, wherein:
R³ is a substituted or unsubstituted ring selected from aryl and heteroaryl; and
PG¹ and PG³ are independently suitable amine protecting groups.

73. (canceled)

74. A compound of formula 14 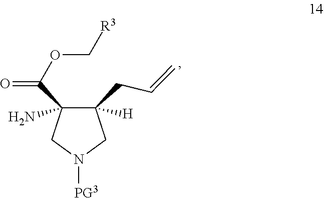
or a salt thereof, wherein:
R³ is a substituted or unsubstituted ring selected from aryl and heteroaryl; and
PG³ is a suitable amine protecting group.

75. (canceled)

76. A compound of formula 13 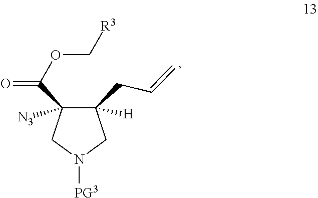
or a salt thereof, wherein:
R³ is a substituted or unsubstituted ring selected from aryl and heteroaryl; and
PG³ is a suitable amine protecting group.

77-83. (canceled)

84. A compound of formula 20c 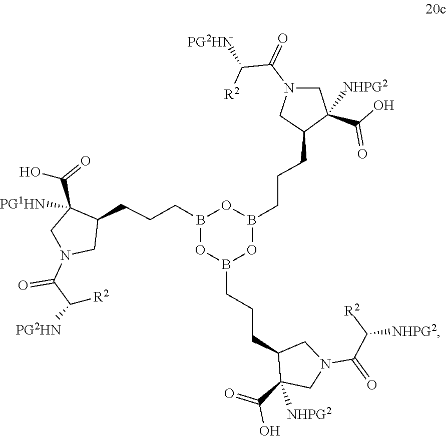
or a salt thereof, wherein:
R² is H, (C₁-C₆)alkyl, or —CH₂OH; and
PG¹ and PG² are independently suitable amine protecting groups.

85-94. (canceled)
